(19) United States
(12) Patent Application Publication
(10) Pub. No.: US 2008/0015184 A1
(43) Pub. Date: Jan. 17, 2008

(54) Urea Substituted Imidazopyridines, Imidazoquinolines, and Imidazonaphthyridines

(75) Inventors: Tushar A. Kshirsagar, Woodbury, MN (US); George W. Griesgraber, Eagan, MN (US); Sarah C. Johannessen, Minneapolis, MN (US); Azim A. Celebi, Clark, NJ (US); Philip D. Heppner, Forest Lake, MN (US); Gregory D. Lundquist Jr., Eagan, MN (US); David T. Amos, St Paul, MN (US); Bernhard M. ZImmermann, Eagan, MN (US); Scott E. Langer, Woodbury, MN (US)

Correspondence Address: 3M INNOVATIVE PROPERTIES COMPANY, PO BOX 33427, ST. PAUL, MN, 55133-3427, US

(73) Assignee: 3M INNOVATIVE PROPERTIES COMPANY, St. Paul, MN (US)

(21) Appl. No.: 11/570,567

(22) PCT Filed: Jun. 14, 2005
(86) PCT No.: PCT/US05/20895

§ 371 Date: Dec. 13, 2006

Related U.S. Application Data
(60) Provisional application No. 60/579,352, filed on Jun. 14, 2004.

(51) Int. Cl. A61K 31/437; A61K 31/496; A61K 31/5377; A61K 31/541; A61K 31/551; A61P 31/12; A61P 35/00; C07D 243/08; C07D 279/12; C07D 401/14; C07D 413/14; C07D 471/04

(52) U.S. Cl. 514/218; 514/228.5; 514/232.8; 514/253.03; 514/293; 540/575; 544/126; 544/361; 544/58.6; 546/82

(57) ABSTRACT

Imidazopyridine, imidazoquinoline, and imidazonaphthyridine compounds having a urea substituent at the 2-position, pharmaceutical compositions containing the compounds, intermediates, and methods of making and methods of use of these compounds as immunomodulators, for modulating cytokine biosynthesis in animals and in the treatment of diseases including viral and neoplastic diseases are disclosed.

### RELATED APPLICATIONS

The present invention claims priority to U.S. Provisional Application Ser. No. 60/579,352, filed Jun. 14, 2004, which is incorporated herein by reference.

### BACKGROUND OF THE INVENTION

In the 1950's the 1H-imidazo[4,5-c]quinoline ring system was developed, and 1-(6-methoxy-8-quinolinyl)-2-methyl-1H-imidazo[4,5-c]quinoline was synthesized for possible use as an antimalarial agent. Subsequently, syntheses of various substituted 1H-imidazo[4,5-c]quinolines were reported. For example, 1-[2-(4-piperidyl)ethyl]-1H-imidazo[4,5-c]quinoline was synthesized as a possible anticonvulsant and cardiovascular agent. Also, several 2-oxoimridazo[4,5-c]quinolines have been reported.

Certain 1H-imidazo[4,5-c]quinolin-4-amines and 1- and 2-substituted derivatives thereof were later found to be useful as antiviral agents, bronchodilators and immunomodulators. Subsequently, certain substituted 1H-imidazo[4,5-c]pyridin-4-amine, quinolin-4-amine, tetrahydroquinolin-4-amine, naphthyridin-4-amine, and tetrahydronaphthyridine-4-amine compounds as well as certain analogous thiazolo and oxazolo compounds were synthesized and found to be useful as immune response modifiers, rendering them useful in the treatment of a variety of disorders. There continues to be interest in and a need for compounds that have the ability to modulate the immune response, by induction of cytokine biosynthesis or other mechanisms.

### SUMMARY OF THE INVENTION

It has now been found that certain urea substituted imidazopyridine, imidazoquinoline, and imidazonaphthyridine compounds modulate cytokine biosynthesis. Such compounds are of the following Formula I: 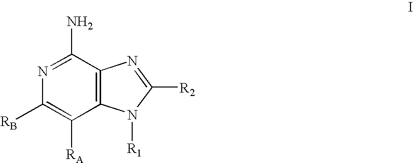
wherein R₁, R₂, R_{A}, and R_{B} are as defined below; and pharmaceutically acceptable salts thereof.

The compounds of Formula I are useful, for example, as immune response modifiers (IRMs) due to their ability to modulate cytokine biosynthesis (e.g., induce or inhibit the biosynthesis or production of at least one cytokine) and otherwise modulate the immune response when administered to animals. Compounds can be tested, for example, using the test procedures described in the Examples Section. Compounds can be tested for induction of cytokine biosynthesis by incubating human PBMC in a culture with the compound(s) at a concentration range of 30 to 0.014 μM and analyzing for interferon (α) or tumor necrosis factor (α) in the culture supernatant. Compounds can be tested for inhibition of cytokine biosynthesis by incubating mouse macrophage cell line Raw 264.7 in a culture with the compound(s) at a single concentration of, for example, 5 μM and analyzing for tumor necrosis factor (α) in the culture supernatant. The ability to modulate cytokine biosynthesis, for example, induce the biosynthesis of at least one cytokine, makes the compounds useful in the treatment of a variety of conditions such as viral diseases and neoplastic diseases, that are responsive to such changes in the immune response.

The invention further provides pharmaceutical compositions containing an effective amount of a compound of Formula I and methods of inducing cytokine biosynthesis in an animal, treating a viral infection and/or treating a neoplastic disease in an animal by administering an effective amount of a compound of Formula I to the animal.

In another aspect, the invention provides methods of synthesizing the compounds of Formula I and intermediates useful in the synthesis of these compounds.

As used herein, “a”, “an”, “the”, “at least one”, and “one or more” are used interchangeably.

The terms “comprising” and variations thereof do not have a limiting meaning where these terms appear in the description and claims.

The above summary of the present invention is not intended to describe each disclosed embodiment or every implementation of the present invention. The description that follows more particularly exemplifies illustrative embodiments. Guidance is also provided herein through lists of examples, which can be used in various combinations. In each instance, the recited list serves only as a representative group and should not be interpreted as an exclusive or exhaustive list.

### DETAILED DESCRIPTION OF ILLUSTRATIVE EMBODIMENTS OF THE INVENTION

The present invention provides compounds of the following Formulas I through VIII: 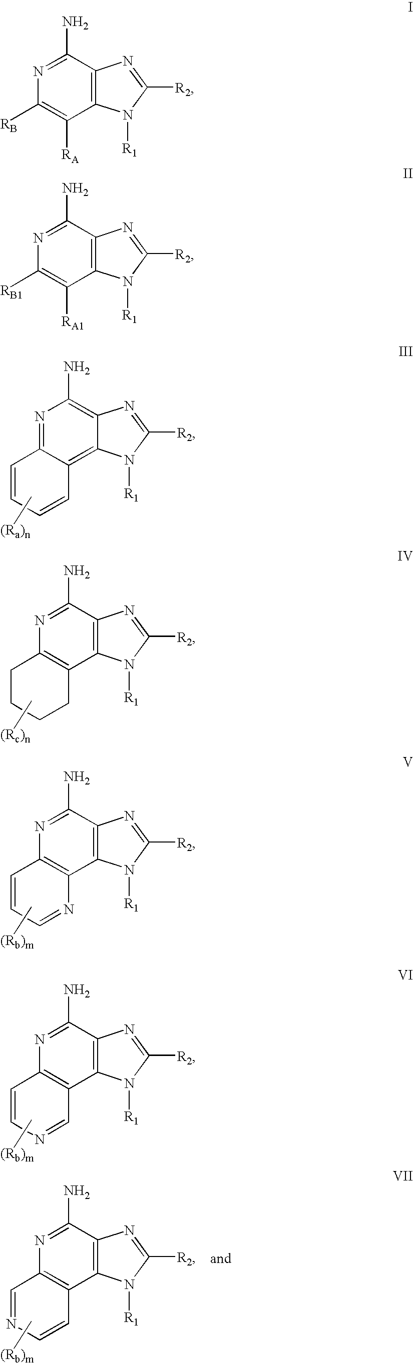 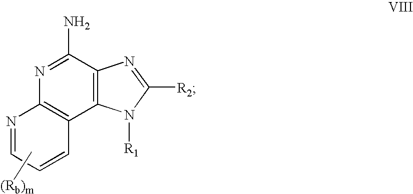
intermediates of the following Formulas X through XVII, some of which are also immune response modifiers: 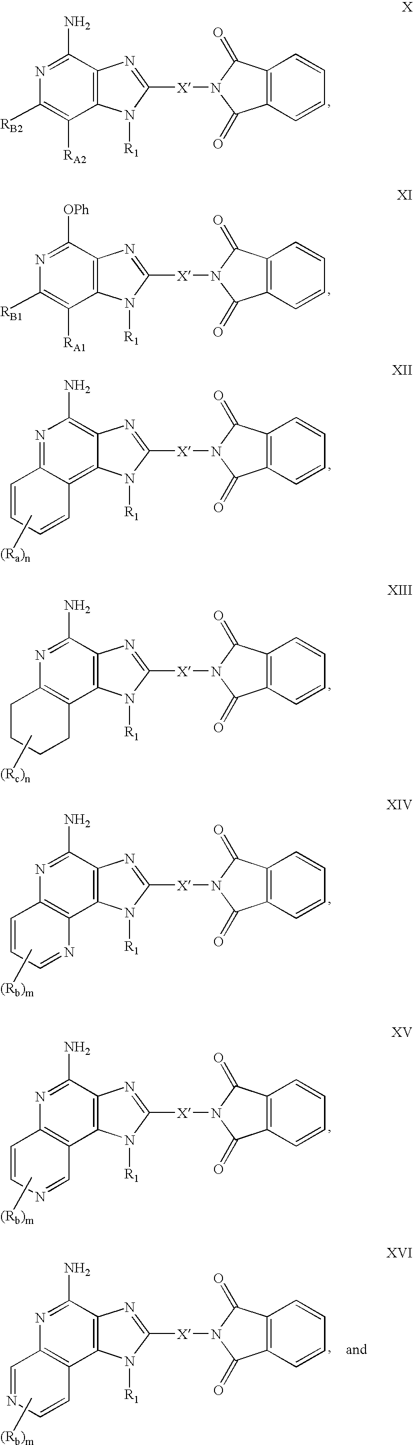 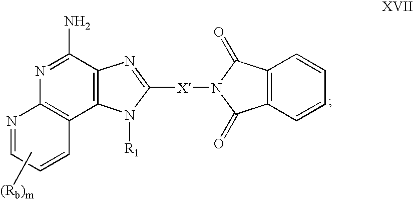
and prodrugs of the following Formula XVIII: 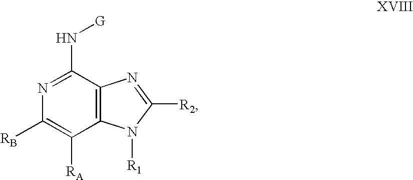
wherein R₁, R₂, R_{A}, R_{B}, R_{A1}, R_{B1}, R_{A2}, R_{B2}, Rₐ, R_{b}, R_{c}, G, Ph, X′, n, and m are as defined below.

In one embodiment, the present invention provides an imidazopyridine, imidazoquinoline and imidazonaphthyridine compound of the following Formula I: 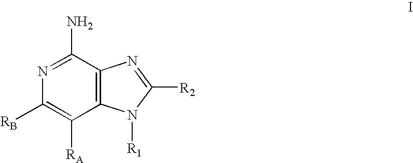
wherein:

R₂ is selected from the group consisting of:
—X′—N(R₈ₐ)—C(R₆)—N(R₈ₐ)—W—R₂₋₁,
—X′—N(R₈ₐ)—C(R₆)—N(OR₈ₐ)—R₂₋₁, 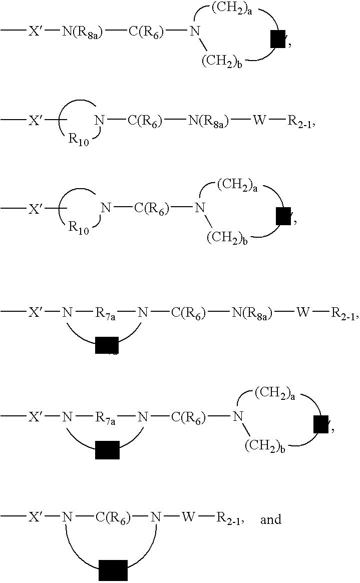
—X′—N(R₈ₐ)—C(R₆)—O—R₂₋₁;

X′ is selected from the group consisting of C₁₋₄ alkylene and C₂₋₄ alkenylene;

R₂₋₁ is selected from the group consisting of hydrogen, C₁₋₄ alkyl, C₂₋₄ alkenyl, C₂₋₄ alkynyl, aryl, arylC₁₋₄ alkylenyl, aryloxyC₁₋₄ alkylenyl, C₁₋₄ alkylarylenyl, heteroaryl, heteroarylC₁₋₄ alkylenyl, heteroaryloxyC₁₋₄ alkylenyl, C₁₋₄ alkylheteroarylenyl, and heterocyclyl wherein the C₁₋₄ alkyl, C₂₋₄ alkenyl, C₂₋₄ alkynyl, aryl, arylC₂₋₄ alkylenyl, aryloxyC₁₋₄ alkylenyl, C₁₋₄ alkylarylenyl, heteroaryl, heteroarylC₁₋₄ alkylenyl, heteroaryloxyC₁₋₄ alkylenyl, C₁₋₄ alkylheteroarylenyl, and heterocyclyl groups are unsubstituted or substituted by one or more substituents independently selected from the group consisting of C₁₋₄ alkyl, C₁₋₄ alkoxy, C₁₋₄ alkanoyl, C₁₋₄ alkoxycarbonyl, hydroxyC₁₋₄ alkylenyl, haloC₁₋₄ alkylenyl, haloC₁₋₄ alkyleneoxy, halogen, nitro, hydroxy, mercapto, cyano, amino, C₁₋₄ alkylamino, di(C₁₋₄ alkyl)amino, and in the case of C₁₋₄ alkyl, C₂₋₄ alkenyl, C₂₋₄ alkynyl, and heterocyclyl, oxo;

A′ is selected from the group consisting of —O—, —C(O)—, —CH₂—, —S(O)₀₋₂—, —NH—, and —N(C₁₋₄ alkyl)-;

R₇ₐ is C₂₋₄ alkylene;

R₈ₐ is selected from the group consisting of hydrogen and C₁₋₄ alkyl;

R_{A} and R_{B} are independently selected from the group consisting of:
hydrogen,
halogen,
alkyl,
alkenyl,
alkoxy,
alkylthio, and
—N(R₉)₂;

or R_{A} and R_{B} taken together form either a fused aryl ring that is unsubstituted or substituted by one or more Rₐ groups, or a fused 5 to 7 membered saturated ring that is unsubstituted or substituted by one or more R_{c} groups;

or R_{A} and R_{B} taken together form a fused heteroaryl or 5 to 7 membered saturated ring containing one heteroatom selected from the group consisting of N and S, wherein the heteroaryl ring is unsubstituted or substituted by one or more R_{b} groups, and the 5 to 7 membered saturated ring is unsubstituted or substituted by one or more R_{c} groups;

Rₐ is selected from the group consisting of halogen, alkyl, haloalkyl, alkoxy, and —N(R₉)₂;

R_{b} is selected from the group consisting of halogen, hydroxy, alkyl, haloalkyl, alkoxy, and —N(R₉)₂;

R_{c} is selected from the group consisting of halogen, hydroxy, alkyl, alkenyl, haloalkyl, alkoxy, alkylthio, and —N(R₉)₂;

R₁ is selected from the group consisting of:
—R₄,
—X—R₄,
—X—Y—R₄,
—X—Y—X—Y—R₄, and
—X—R₅;

X is selected from the group consisting of alkylene, arylene, heteroarylene, and heterocyclylene wherein the alkylene group can be optionally interrupted or terminated by arylene, heteroarylene or heterocyclylene and optionally interrupted by one or more —O— groups;

Y is selected from the group consisting of:
—S(O)₀₋₂—,
—C(R₆)—,
—C(R₆)—O—,
—O—C(R₆)—,
—O—C(O)—O—,
—N(R₈)-Q-,
—O—C(R₆)—N(R₈)—,
—C(R₆)—N(OR₉)—,

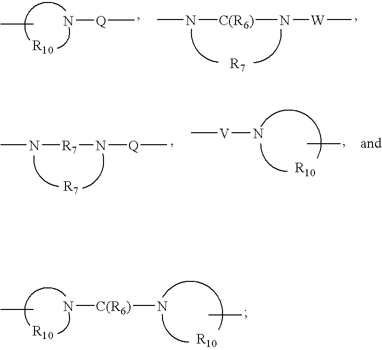

R₄ is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the alkyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups can be unsubstituted or substituted by one or more substituents independently selected from the group consisting of alkyl, alkoxy, hydroxyalkylenyl, haloalkylenyl, haloalkyleneoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, (dialkylamino)alkyleneoxy, and in the case of alkyl and heterocyclyl, oxo;

R₅ is selected from the group consisting of:

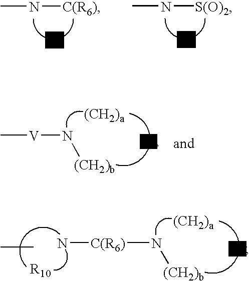

R₆ is selected from the group consisting of ═O and ═S;

R₇ is C₂₋₇ alkylene;

R₈ is selected from the group consisting of hydrogen, alkyl, alkoxyalkylenyl, and arylalkylenyl;

R₉ is selected from the group consisting of hydrogen and alkyl;

R₁₀ is C₃₋₈ alkylene;

A is selected from the group consisting of —O—, —C(O)—, —CH₂—, —S(O)₀₋₂—, and —N(R₄)—;

Q is selected from the group consisting of a bond, —C(R₆)—, —C(R₆)—C(R₆)—, —S(O)₂—, —C(R₆)—N(R₈)—W—, —S(O)₂—N(R₈)—, —C(R₆)—O—, —C(R₆)—S—, and —C(R₆)—N(OR^{s})—;

V is selected from the group consisting of —O—C(R₆)— and —N(R₈)—C(R₆)—;

W is selected from the group consisting of a bond, —C(O)—, and —S(O)₂—; and

a and b are independently integers from 1 to 6 with the proviso that a+b is ≦7;

with the proviso that when R_{A} and R_{B} taken together form a ring, and X is interrupted with one —O— group, then Y is other than —S(O)₀₋₂—; and

with the further proviso that when R_{A} and R_{B} are independently hydrogen, halogen, alkyl, alkenyl, alkoxy, alkylthio, or —N(R₉)₂, and R₂ is selected from the group consisting of: 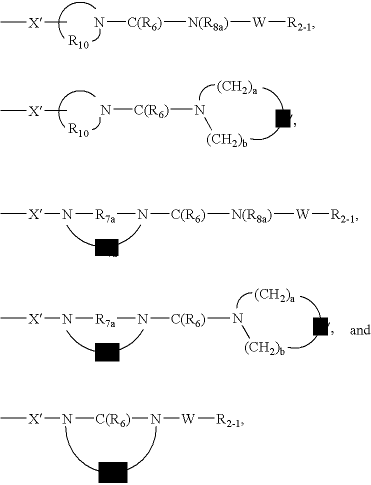
then X is not interrupted with one or more —O— groups and Y is other than —S(O)O₀₋₂—;
or a pharmaceutically acceptable salt thereof.

In one embodiment, the present invention also provides an imidazopyridine compound of the following Formula II: 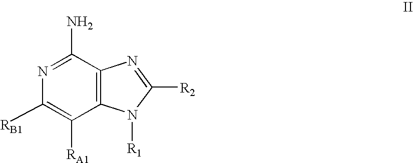
wherein:

R₂ is selected from the group consisting of:
—X′—N(R₈ₐ)—C(R₆)—N(R₈ₐ)—W—R₂₋₁,
—X′—N(R₈ₐ)—C(R₆)—N(OR₈ₐ)—R₂₋₁, 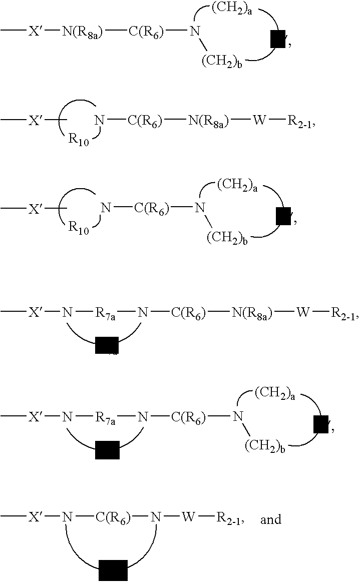
—X′—N(R₈ₐ)—C(R₆)—O—R₂₋₁;

X′ is selected from the group consisting of C₁₋₄ alkylene and C₂₋₄ alkenylene;

R₂₋₁ is selected from the group consisting of hydrogen, C₁₋₄ alkyl, C₂₋₄ alkenyl, C₂₋₄ alkynyl, aryl, arylC₁₋₄ alkylenyl, aryloxyC₁₋₄ alkylenyl, C₁₋₄ alkylarylenyl, heteroaryl, heteroarylC₁₋₄ alkylenyl, heteroaryloxyC₁₋₄ alkylenyl, C₁₋₄ alkylheteroarylenyl, and heterocyclyl wherein the C₁₋₄ alkyl, C₂₋₄ alkenyl, C₂₋₄ alkynyl, aryl, arylC₁₋₄ alkylenyl, aryloxyC₁₋₄ alkylenyl, C₁₋₄ alkylarylenyl, heteroaryl, heteroarylC₁₋₄ alkylenyl, heteroaryloxyC₁₋₄ alkylenyl, C₁₋₄ alkylheteroarylenyl, and heterocyclyl groups are unsubstituted or substituted by one or more substituents independently selected from the group consisting of C₁₋₄ alkyl, C₁₋₄ alkoxy, C₁₋₄ alkanoyl, C₁₋₄ alkoxycarbonyl, hydroxyC₁₋₄ alkylenyl, haloC₁₋₄ alkylenyl, haloC₁₋₄ alkyleneoxy, halogen, nitro, hydroxy, mercapto, cyano, amino, C₁₋₄ alkylamino, di(C₁₋₄ alkyl)amino, and in the case of C₁₋₄ alkyl, C₂₋₄ alkenyl, C₂₋₄ alkynyl, and heterocyclyl, oxo;

A′ is selected from the group consisting of —O—, —C(O)—, —CH₂—, —S(O)₀₋₂—, —NH—, and —N(C₁₋₄ alkyl)-;

R₇ₐ is C₂₋₄ alkylene;

R₈ₐ is selected from the group consisting of hydrogen and C₁₋₄ alkyl;

R_{A1} and R_{B1}, are independently selected from the group consisting of:
hydrogen,
halogen,
alkyl,
alkenyl,
alkoxy,
alkylthio, and
—N(R₉)₂;

R₁ is selected from the group consisting of:
—R₄,
—X—R₄,
—X—Y—R₄,
—X—Y—X—Y—R₄, and
—X—R₅;

X is selected from the group consisting of alkylene, arylene, heteroarylene, and heterocyclylene wherein the alkylene group can be optionally interrupted or terminated by arylene, heteroarylene or heterocyclylene and optionally interrupted by one or more —O— groups;

Y is selected from the group consisting of:
—S(O)₀₋₂—,
—C(R₆)—,
—C(R₆)—O—,
—O—C(R₆)—,
—O—C(O)—O—,
—N(R₈)-Q-,
—O—C(R₆)—N(R₈)—,
—C(R₆)—N(OR₉)—,

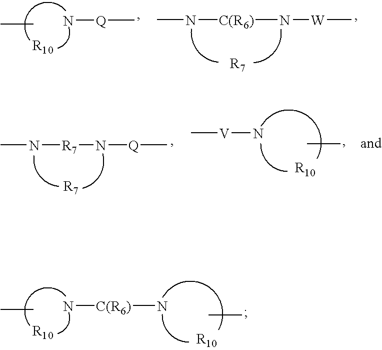

R₄ is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the alkyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups can be unsubstituted or substituted by one or more substituents independently selected from the group consisting of alkyl, alkoxy, hydroxyalkylenyl, haloalkylenyl, haloalkyleneoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, (dialkylamino)alkyleneoxy, and in the case of alkyl and heterocyclyl, oxo;

R₅ is selected from the group consisting of:

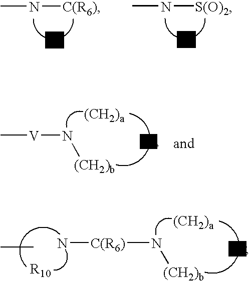

R₆ is selected from the group consisting of ═O and ═S;

R₇ is C₂₋₇ alkylene;

R₈ is selected from the group consisting of hydrogen, alkyl, alkoxyalkylenyl, and arylalkylenyl;

R₉ is selected from the group consisting of hydrogen and alkyl;

R₁₀ is C₃₋₈ alkylene;

A is selected from the group consisting of —O—, —C(O)—, —CH₂—, —S(O)₀₋₂—, and —N(R₄)—;

Q is selected from the group consisting of a bond, —C(R₆)—, —C(R₆)—C(R₆)—, —S(O)₂—, —C(R₆)—N(R₈)—W—, —S(O)₂—N(R₈)—, —C(R₆)—O—, —C(R₆)—S—, and —C(R₆)—N(OR₉)—;

V is selected from the group consisting of —O—C(R₆)— and —N(R₈)—C(R₆)—;

W is selected from the group consisting of a bond, —C(O)—, and —S(O)₂—; and

a and b are independently integers from 1 to 6 with the proviso that a+b is ≦7;

with the proviso that when R₂ is selected from the group consisting of: 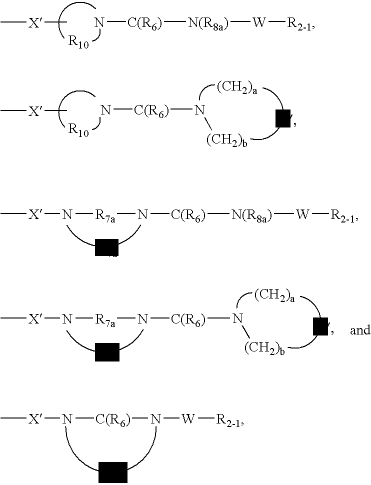
then X is not interrupted with one or more —O— groups and Y is other than —S(O)₀₋₂—;
or a pharmaceutically acceptable salt thereof.

In one embodiment, the present invention also provides an imidazoquinoline compound of the following Formula III: 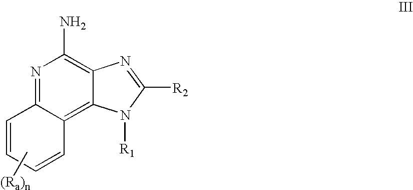
wherein:

R₂ is selected from the group consisting of:
—X′—N(R₈ₐ)—C(R₆)—N(R₈ₐ)—W—R₂₋₁,
—X′—N(R₈ₐ)—C(R₆)—N(OR₈ₐ)—R₂₋₁, 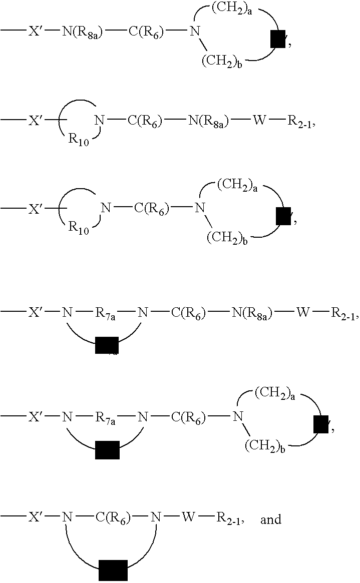
—X′—N(R₈ₐ)—C(R₆)—O—R₂₋₁;

X′ is selected from the group consisting of C₁₋₄ alkylene and C₂₋₄ alkenylene;

R₂₋₁ is selected from the group consisting of hydrogen, C₁₋₄ alkyl, C₂₋₄ alkenyl, C₂₋₄ alkynyl, aryl, arylC₁₋₄ alkylenyl, aryloxyC₁₋₄ alkylenyl, C₁₋₄ alkylarylenyl, heteroaryl, heteroarylC₁₋₄ alkylenyl, heteroaryloxyC₁₋₄ alkylenyl, C₁₋₄ alkylheteroarylenyl, and heterocyclyl wherein the C₁₋₄ alkyl, C₂₋₄ alkenyl, C₂₋₄ alkynyl, aryl, arylC₁₋₄ alkylenyl, aryloxyC₁₋₄ alkylenyl, C₁₋₄ alkylarylenyl, heteroaryl, heteroarylC₁₋₄ alkylenyl, heteroaryloxyC₁₋₄ alkylenyl, C₁₋₄ alkylheteroarylenyl, and heterocyclyl groups are unsubstituted or substituted by one or more substituents independently selected from the group consisting of C₁₋₄ alkyl, C₁₋₄ alkoxy, C₁₋₄ alkanoyl, C₁₋₄ alkoxycarbonyl, hydroxyC₁₋₄ alkylenyl, haloC₁₋₄ alkylenyl, haloC₁₋₄ alkyleneoxy, halogen, nitro, hydroxy, mercapto, cyano, amino, C₁₋₄ alkylamino, di(C₁₋₄ alkyl)amino, and in the case of C₁₋₄ alkyl, C₂₋₄ alkenyl, C₂₋₄ alkynyl, and heterocyclyl, oxo;

A′ is selected from the group consisting of —O—, —C(O)—, —CH₂—, —S(O)₀₋₂—, —NH—, and —N(C₁₋₄ alkyl)-;

R₇ₐ is C₂₋₄ alkylene;

R₈ₐ is selected from the group consisting of hydrogen and C₁₋₄ alkyl;

Rₐ is selected from the group consisting of halogen, alkyl, haloalkyl, alkoxy, and —N(R₉)₂;

n is an integer from 0 to 4;

R₁ is selected from the group consisting of:
—R₄,
—X—R₄,
—X—Y—R₄,
—X—Y—X—Y—R₄, and
—X—R₅;

X is selected from the group consisting of alkylene, arylene, heteroarylene, and heterocyclylene wherein the alkylene group can be optionally interrupted or terminated by arylene, heteroarylene or heterocyclylene and optionally interrupted by one or more —O— groups;

Y is selected from the group consisting of:
—S(O)₀₋₂—,
—C(R₆)—,
—C(R₆)—O—,
—O—C(R₆)—,
O—C(O)—O—,
—N(R₈)-Q-,
—O—C(R₆)—N(R₉)—,
—C(R₆)—N(OR₉)—,

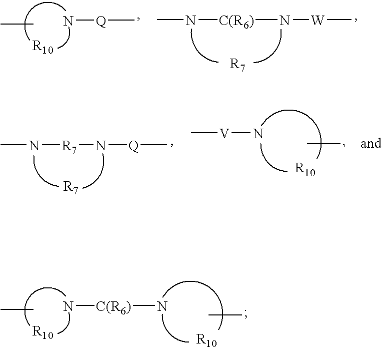

R₄ is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the alkyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups can be unsubstituted or substituted by one or more substituents independently selected from the group consisting of alkyl, alkoxy, hydroxyalkylenyl, haloalkylenyl, haloalkyleneoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, (dialkylamino)alkyleneoxy, and in the case of alkyl and heterocyclyl, oxo;

R₅ is selected from the group consisting of:

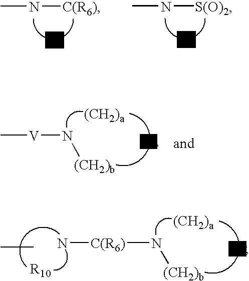

R₆ is selected from the group consisting of ═O and ═S;

R₇ is C₂₋₇ alkylene;

R₈ is selected from the group consisting of hydrogen, alkyl, alkoxyalkylenyl, and arylalkylenyl;

R₉ is selected from the group consisting of hydrogen and alkyl;

R₁₀ is C₃₋₈ alkylene;

A is selected from the group consisting of —O—, —C(O)—, —CH₂—, —S(O)₀₋₂—, and —N(R₄)—;

Q is selected from the group consisting of a bond, —C(R₆)—, —C(R₆)—C(R₆)—, —S(O)₂—, —C(R₆)—N(R₈)—W—, —S(O)₂—N(R₈)—, —C(R₆)—O—, —C(R₆)—S—, and —C(R₆)—N(OR₉)—;

V is selected from the group consisting of —O—C(R₆)— and —N(R₈)—C(R₆)—;

W is selected from the group consisting of a bond, —C(O)—, and —S(O)₂—; and

a and b are independently integers from 1 to 6 with the proviso that a+b is ≦7;

with the proviso that when X is interrupted with one —O— group, then Y is other than —S(O)₀₋₂—;

or a pharmaceutically acceptable salt thereof.

In one embodiment, the present invention also provides a 6,7,8,9-tetrahydroimidazoquinoline compound of the following Formula IV: 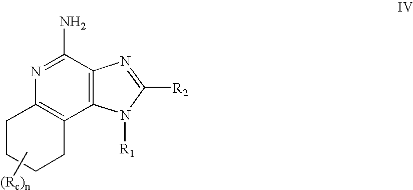
wherein:

R₂ is selected from the group consisting of:
—X′—N(R₈ₐ)—C(R₆)—N(R₈ₐ)—W—R₂₋₁,
—X′—N(R₈ₐ)—C(R₆)—N(OR₈ₐ)—R₂₋₁, 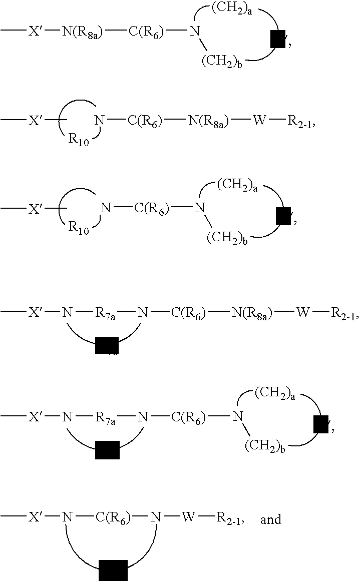
—X′—N(R₈ₐ)—C(R₆)—O—R₂₋₁;

X′ is selected from the group consisting of C₁₋₄ alkylene and C₂₋₄ alkenylene;

R₂₋₁ is selected from the group consisting of hydrogen, C₁₋₄ alkyl, C₂₋₄ alkenyl, C₂₋₄ alkynyl, aryl, arylC₁₋₄ alkylenyl, aryloxyC₁₋₄ alkylenyl, C₁₋₄ alkylarylenyl, heteroaryl, heteroarylC₁₋₄ alkylenyl, heteroaryloxyC₁₋₄ alkylenyl, C₁₋₄ alkylheteroarylenyl, and heterocyclyl wherein the C₁₋₄ alkyl, C₂₋₄ alkenyl, C₂₋₄ alkynyl, aryl, arylC₁₋₄ alkylenyl, aryloxyC₁₋₄ alkylenyl, C₁₋₄ alkylarylenyl, heteroaryl, heteroarylC₁₋₄ alkylenyl, heteroaryloxyC₁₋₄ alkylenyl, C₁₋₄ alkylheteroarylenyl, and heterocyclyl groups are unsubstituted or substituted by one or more substituents independently selected from the group consisting of C₁₋₄ alkyl, C₁₋₄ alkoxy, C₁₋₄ alkanoyl, C₁₋₄ alkoxycarbonyl, hydroxyC₁₋₄ alkylenyl, haloC₁₋₄ alkylenyl, haloC₁₋₄ alkyleneoxy, halogen, nitro, hydroxy, mercapto, cyano, amino, C₁₋₄ alkylamino, di(C₁₋₄ alkyl)amino, and in the case of C₁₋₄ alkyl, C₂₋₄ alkenyl, C₂₋₄ alkynyl, and heterocyclyl, oxo;

A′ is selected from the group consisting of —O—, —C(O)—, —CH₂—, —S(O)₀₋₂—, —NH—, and —N(C₁₋₄ alkyl)-;

R₇ₐ is C₂₋₄ alkylene;

R₈ₐ is selected from the group consisting of hydrogen and C₁₋₄ alkyl;

R_{c} is selected from the group consisting of halogen, hydroxy, alkyl, alkenyl, haloalkyl, alkoxy, alkylthio, and —N(R₉)₂;

n is an integer from 0 to 4;

R₁ is selected from the group consisting of:
—R₄,
—X—R₄,
—X—Y—R₄,
—X—Y—X—Y—R₄, and
—X—R₅;

X is selected from the group consisting of alkylene, arylene, heteroarylene, and heterocyclylene wherein the alkylene group can be optionally interrupted or terminated by arylene, heteroarylene or heterocyclylene and optionally interrupted by one or more —O— groups;

Y is selected from the group consisting of:
—S(O)₀₋₂—,
—C(R₆)—,
—C(R)—O—,
—O—C(R)—,
—O—C(O)—O—,
—N(R₈)-Q-,
—O—C(R₆)—N(R₈)—,
—C(R₆)—N(OR₉)—,

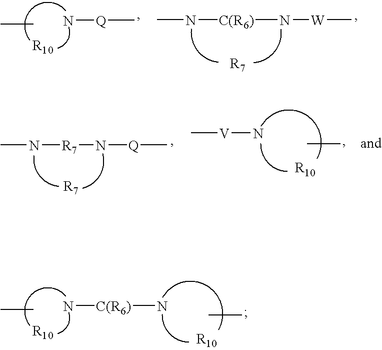

R₄ is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the alkyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups can be unsubstituted or substituted by one or more substituents independently selected from the group consisting of alkyl, alkoxy, hydroxyalkylenyl, halo alkylenyl, halo alkyleneoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, (dialkylamino)alkyleneoxy, and in the case of alkyl and heterocyclyl, oxo;

R₅ is selected from the group consisting of:

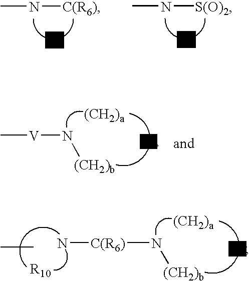

R₆ is selected from the group consisting of ═O and ═S;

R₇ is C₂₋₇ alkylene;

R₈ is selected from the group consisting of hydrogen, alkyl, alkoxyalkylenyl, and arylalkylenyl;

R₉ is selected from the group consisting of hydrogen and alkyl;

R₁₀ is C₃₋₈ alkylene;

A is selected from the group consisting of —O—, —C(O)—, —CH₂—, —S(O)₀₋₂—, and —N(R₄)—;

Q is selected from the group consisting of a bond, —C(R₆)—, —C(R₆)—C(R₆)—, —S(O)₂—, —C(R₆)—N(R₈)—W—, —S(O)₂—N(R₈)—, —C(R₆)—O—, —C(R₆)—S—, and —C(R₆)—N(OR^{s})—;

V is selected from the group consisting of —O—C(R₆)— and —N(R₉)—C(R₆)—;

W is selected from the group consisting of a bond, —C(O)—, and —S(O)₂—; and

a and b are independently integers from 1 to 6 with the proviso that a+b is ≦7;

with the proviso that when X is interrupted with one —O— group, then Y is other than —S(O)₀₋₂—;

or a pharmaceutically acceptable salt thereof.

In one embodiment, the present invention also provides an imidazonaphthyridine compound selected from the group consisting of the following Formulas V, VI, VII, and VIII: 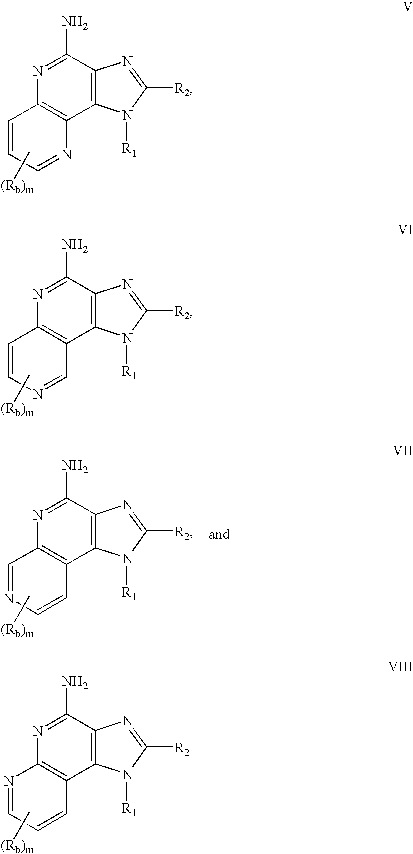
wherein:

R₂ is selected from the group consisting of:
—X′—N(R₈ₐ)—C(R₆)—N(R₈ₐ)—W—R₂₋₁,
—X′—N(R₈ₐ)—C(R₆)—N(OR₈ₐ)—R₂₋₁, 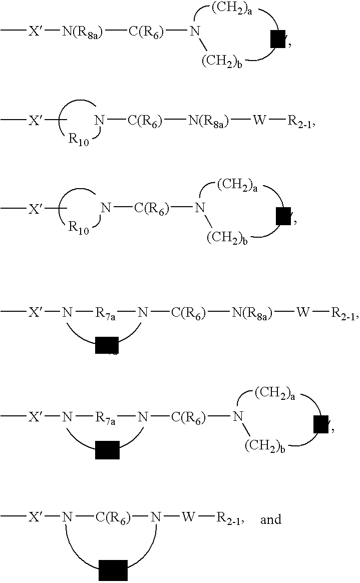
—X′—N(R₈ₐ)—C(R₆)—O—R₂₋₁;

X′ is selected from the group consisting of C₁₋₄ alkylene and C₂₋₄ alkenylene;

R₂₋₁ is selected from the group consisting of hydrogen, C₁₋₄ alkyl, C₂₋₄ alkenyl, C₂₋₄ alkynyl, aryl, arylC₁₋₄ alkylenyl, aryloxyC₁₋₄ alkylenyl, C₁₋₄ alkylarylenyl, heteroaryl, heteroarylC₁₋₄ alkylenyl, heteroaryloxyC₁₋₄ alkylenyl, C₁₋₄ alkylheteroarylenyl, and heterocyclyl wherein the C₁₋₄ alkyl, C₂₋₄ alkenyl, C₂₋₄ alkynyl, aryl, arylC₁₋₄ alkylenyl, aryloxyC₁₋₄ alkylenyl, C₁₋₄ alkylarylenyl, heteroaryl, heteroarylC₁₋₄ alkylenyl, heteroaryloxyC₁₋₄ alkylenyl, C₁₋₄ alkylheteroarylenyl, and heterocyclyl groups are unsubstituted or substituted by one or more substituents independently selected from the group consisting of C₁₋₄ alkyl, C₁₋₄ alkoxy, C₁₋₄ alkanoyl, C₁₋₄ alkoxycarbonyl, hydroxyC₁₋₄ alkylenyl, haloC₁₋₄ alkylenyl, haloC₁₋₄ alkyleneoxy, halogen, nitro, hydroxy, mercapto, cyano, amino, C₁₋₄ alkylamino, di(C₁₋₄ alkyl)amino, and in the case of C₁₋₄ alkyl, C₂₋₄ alkenyl, C₂₋₄ alkynyl, and heterocyclyl, oxo;

A′ is selected from the group consisting of —O—, —C(O)—, —CH₂—, —S(O)₀₋₂—, —NH—, and —N(C₁₋₄ alkyl)-;

R₇ₐ is C₂₋₄ alkylene;

R₈ₐ is selected from the group consisting of hydrogen and C₁₋₄ alkyl;

R_{b} is selected from the group consisting of halogen, hydroxy, alkyl, haloalkyl, alkoxy, and —N(R₉)₂;

m is an integer from 0 to 3;

R₁ is selected from the group consisting of:
—R₄,
—X—R₄,
—X—Y—R₄,
—X—Y—X—Y—R₄, and
—X—R₅;

X is selected from the group consisting of alkylene, arylene, heteroarylene, and heterocyclylene wherein the alkylene group can be optionally interrupted or terminated by arylene, heteroarylene or heterocyclylene and optionally interrupted by one or more —O— groups;

Y is selected from the group consisting of:
—S(O)₀₋₂—,
—C(R₆)—,
—C(R₆)—O—,
—O—C(R₆)—,
—O—C(O)—O—,
—N(R₈)-Q-,
—O—C(R₆)—N(R₈)—,
—C(R₆)—N(OR₉)—,

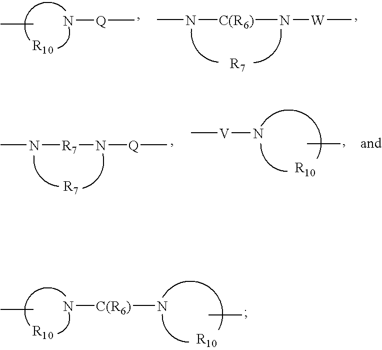

R₄ is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the alkyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups can be unsubstituted or substituted by one or more substituents independently selected from the group consisting of alkyl, alkoxy, hydroxyalkylenyl, haloalkylenyl, haloalkyleneoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, (dialkylamino)alkyleneoxy, and in the case of alkyl and heterocyclyl, oxo;

R₅ is selected from the group consisting of:

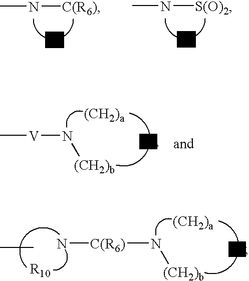

R₆ is selected from the group consisting of ═O and ═S;

R₇ is C₂₋₇ alkylene;

R₈ is selected from the group consisting of hydrogen, alkyl, alkoxyalkylenyl, and arylalkylenyl;

R₉ is selected from the group consisting of hydrogen and alkyl;

R₁₀ is C₃₋₈ alkylene;

A is selected from the group consisting of —O—, —C(O)—, —CH₂—, —S(O)O₀₋₂—, and —N(R₄)—;

Q is selected from the group consisting of a bond, —C(R₆)—, —C(R₆)—C(R₆)—, —S(O)₂—, —C(R₆)—N(R₈)—W—, —S(O)₂—N(R₈)—, —C(R₆)—O—, —C(R₆)—S—, and —C(R₆)—N(OR^{s})—;

V is selected from the group consisting of —O—C(R₆)— and —N(R₈)—C(R₆)—;

W is selected from the group consisting of a bond, —C(O)—, and —S(O)₂—; and

a and b are independently integers from 1 to 6 with the proviso that a+b is ≦7;

with the proviso that when X is interrupted with one —O— group, then Y is other than —S(O)₀₋₂—;

or a pharmaceutically acceptable salt thereof.

The present invention also provides compounds that are useful as intermediates in the synthesis of compounds of Formulas I through VIII. These intermediate compounds include those having the structural Formulas X, XI, XII, XIII, XIV, XV, XVI, and XVII described below, some of which are also immune response modifiers.

In one embodiment, the present invention provides an intermediate compound of the following Formula X: 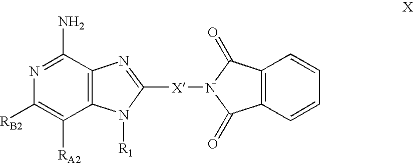
wherein:

X′ is selected from the group consisting of C₁₋₄ alkylene and C₂₋₄ alkenylene;

R_{A2} and R_{B2} taken together form either a fused aryl ring that is unsubstituted or substituted by one or more Rₐ groups, or a fused 5 to 7 membered saturated ring that is unsubstituted or substituted by one or more R_{c} groups;

or R_{A2} and R_{B2} taken together form a fused heteroaryl or 5 to 7 membered saturated ring containing one heteroatom selected from the group consisting of N and S, wherein the heteroaryl ring is unsubstituted or substituted by one or more R_{b} groups, and the 5 to 7 membered saturated ring is unsubstituted or substituted by one or more R_{c} groups;

Rₐ is selected from the group consisting of halogen, alkyl, haloalkyl, alkoxy, and —N(R₉)₂;

R_{b} is selected from the group consisting of halogen, hydroxy, alkyl, haloalkyl, alkoxy, and —N(R₉)₂;

R_{c} is selected from the group consisting of halogen, hydroxy, alkyl, alkenyl, haloalkyl, alkoxy, alkylthio, and —N(R₉)₂;

R₁ is selected from the group consisting of:
—R₄,
—X—R₄,
—X—Y—R₄,
—X—Y—X—Y—R₄, and
—X—R₅;

X is selected from the group consisting of alkylene, arylene, heteroarylene, and heterocyclylene wherein the alkylene group can be optionally interrupted or terminated by arylene, heteroarylene or heterocyclylene and optionally interrupted by one or more —O— groups;

Y is selected from the group consisting of:
—S(O)₀₋₂—,
—C(R₆)—,
—C(R₆)—O—,
—O—C(R₆)—,
—O—C(O)—O—,
—N(R₅)-Q-,
—O—C(R₆)—N(R₉)—,
—C(R₆)—N(OR₉)—,

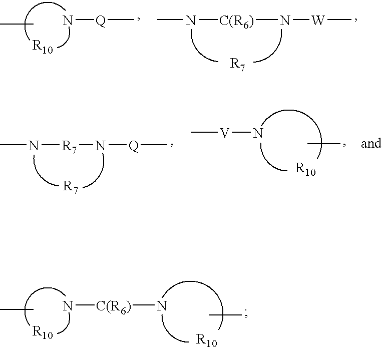

R₄ is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the alkyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups can be unsubstituted or substituted by one or more substituents independently selected from the group consisting of alkyl, alkoxy, hydroxyalkylenyl, haloalkylenyl, haloalkyleneoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, (dialkylamino)alkyleneoxy, and in the case of alkyl and heterocyclyl, oxo;

R₅ is selected from the group consisting of:

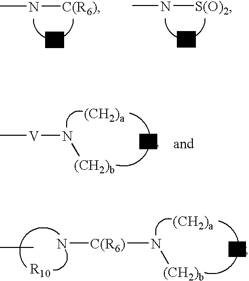

R₆ is selected from the group consisting of ═O and ═S;

R₇ is C₂₋₇ alkylene;

R₈ is selected from the group consisting of hydrogen, alkyl, alkoxyalkylenyl, and arylalkylenyl;

R₉ is selected from the group consisting of hydrogen and alkyl;

R₁₀ is C₃₋₈ alkylene;

A is selected from the group consisting of —O—, —C(O)—, —CH₂—, —S(O)₀₋₂—, and —N(R₄)—;

Q is selected from the group consisting of a bond, —C(R₆)—, —C(R₆)—C(R₆)—, —S(O)₂—, —C(R₆)—N(R₉)—W—, —S(O)₂—N(R₈)—, —C(R₆)—O—, —C(R₆)—S—, and —C(R₆)—N(OR₉)—;

V is selected from the group consisting of —O—C(R^{r})— and —N(R₈)—C(R₆)—;

W is selected from the group consisting of a bond, —C(O)—, and —S(O)₂—; and

a and b are independently integers from 1 to 6 with the proviso that a+b is ≦7;

or a pharmaceutically acceptable salt thereof.

In one embodiment, the present invention provides an intermediate compound of the following Formula XI: 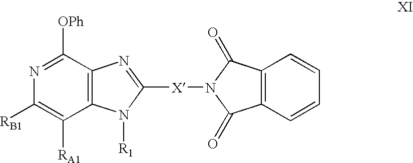
wherein:

Ph is phenyl;

X′ is selected from the group consisting of C₁₋₄ alkylene and C₂₋₄ alkenylene;

R_{A1} and R_{B1} are independently selected from the group consisting of:
hydrogen,
halogen,
alkyl,
alkenyl,
alkoxy,
alkylthio, and
—N(R₉)₂;

R₁ is selected from the group consisting of:
—R₄,
—X—R₄,
—X—Y—R₄,
—X—Y—X—Y—R₄, and
—X—R₅;

X is selected from the group consisting of alkylene, arylene, heteroarylene, and heterocyclylene wherein the alkylene group can be optionally interrupted or terminated by arylene, heteroarylene or heterocyclylene and optionally interrupted by one or more —O— groups;

Y is selected from the group consisting of:
—S(O)₀₋₂—,
—C(R₆)—,
—C(R₆)—O—,
—O—C(R₆)—,
O—C(O)—O—,
—N(R₈)-Q-,
—O—C(R₆)—N(R₈)—,
—C(R₆)—N(OR₉)—,

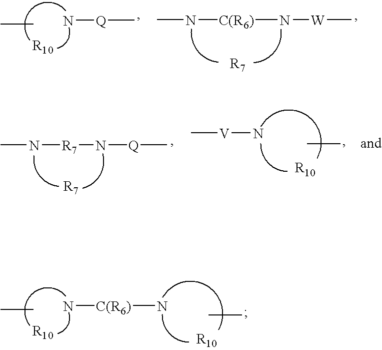

R₄ is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the alkyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups can be unsubstituted or substituted by one or more substituents independently selected from the group consisting of alkyl, alkoxy, hydroxyalkylenyl, haloalkylenyl, haloalkyleneoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, (dialkylamino)alkyleneoxy, and in the case of alkyl and heterocyclyl, oxo;

R₅ is selected from the group consisting of:

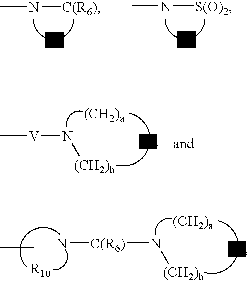

R₆ is selected from the group consisting of ═O and ═S;

R₇ is C₂₋₇ alkylene;

R₈ is selected from the group consisting of hydrogen, alkyl, alkoxyalkylenyl, and arylalkylenyl;

R₉ is selected from the group consisting of hydrogen and alkyl;

R₁₀ is C₃₋₈ alkylene;

A is selected from the group consisting of —O—, —C(O)—, —CH₂—, —S(O)₀₋₂—, and —N(R₄)—;

Q is selected from the group consisting of a bond, —C(R₆)—, —C(R₆)—C(R₆)—, —S(O)₂—, —C(R₆)—N(R₈)—W—, —S(O)₂—N(R₉)—, —C(R₆)—O—, —C(R₆)—S—, and —C(R₆)—N(OR₉)—;

V is selected from the group consisting of —O—C(R₆)— and —N(R₈)—C(R₆)—;

W is selected from the group consisting of a bond, —C(O)—, and —S(O)₂—; and

a and b are independently integers from 1 to 6 with the proviso that a+b is ≦7; or a pharmaceutically acceptable salt thereof.

In one embodiment, the present invention provides an intermediate compound of the following Formula XII: 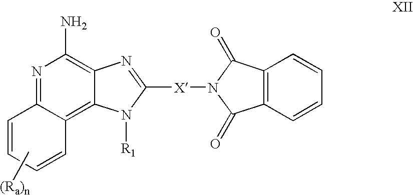
wherein:

X′ is selected from the group consisting of C₁₋₄ alkylene and C₂₋₄ alkenylene;

Rₐ is selected from the group consisting of halogen, alkyl, haloalkyl, alkoxy, and —N(R₉)₂;

n is an integer from 0 to 4;

R₁ is selected from the group consisting of:
—R₄,
—X—R₄,
—X—Y—R₄,
—X—Y—X—Y—R₄, and
—X—R₅;

X is selected from the group consisting of alkylene, arylene, heteroarylene, and heterocyclylene wherein the alkylene group can be optionally interrupted or terminated by arylene, heteroarylene or heterocyclylene and optionally interrupted by one or more —O— groups;

Y is selected from the group consisting of:
—S(O)₀₋₂—,
—C(R₆)—,
—C(R₆)—O—,
—O—C(R₆)—,
—O—C(O)—O—,
—N(R₈)-Q-,
—O—C(R₆)—N(R₈)—,
—C(R₆)—N(OR₉)—,

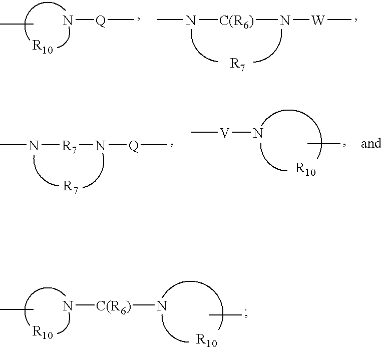

R₄ is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the alkyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups can be unsubstituted or substituted by one or more substituents independently selected from the group consisting of alkyl, alkoxy, hydroxyalkylenyl, haloalkylenyl, haloalkyleneoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, (dialkylamino)alkyleneoxy, and in the case of alkyl and heterocyclyl, oxo;

R₅ is selected from the group consisting of:

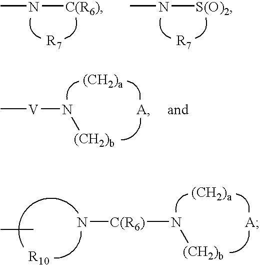

R₆ is selected from the group consisting of ═O and ═S;

R₇ is C₂₋₇ alkylene;

R₈ is selected from the group consisting of hydrogen, alkyl, alkoxyalkylenyl, and arylalkylenyl;

R₉ is selected from the group consisting of hydrogen and alkyl;

R₁₀ is C₃₋₈ alkylene;

A is selected from the group consisting of —O—, —C(O)—, —CH₂—, —S(O)₀₋₂—, and —N(R₄)—;

Q is selected from the group consisting of a bond, —C(R₆)—, —C(R₆)—C(R₆)—, —S(O)₂—, —C(R₆)—N(R₉)—W—, —S(O)₂—N(R₈)—, —C(R₆)—O—, —C(R₆)—S—, and —C(R₆)—N(OR₉)—;

V is selected from the group consisting of —O—C(R₆)— and —N(R₈)—C(R^{r})—;

W is selected from the group consisting of a bond, —C(O)—, and —S(O)₂—; and

a and b are independently integers from 1 to 6 with the proviso that a+b is ≦7; or a pharmaceutically acceptable salt thereof.

In one embodiment, the present invention provides an intermediate compound of the following Formula XIII: 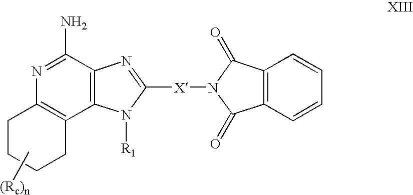
wherein:

X′ is selected from the group consisting of C₁₋₄ alkylene and C₂₋₄ alkenylene;

R_{c} is selected from the group consisting of halogen, hydroxy, alkyl, alkenyl, haloalkyl, alkoxy, alkylthio, and —N(R₉)₂;

n is an integer from 0 to 4;

R₁ is selected from the group consisting of:
—R₄,
—X—R₄,
—X—Y—R₄,
—X—Y—X—Y—R₄, and
—X—R₅;

X is selected from the group consisting of alkylene, arylene, heteroarylene, and heterocyclylene wherein the alkylene group can be optionally interrupted or terminated by arylene, heteroarylene or heterocyclylene and optionally interrupted by one or more —O— groups;

Y is selected from the group consisting of:
—S(O)₀₋₂—,
—C(R₆)—,
—C(R₆)—O—,
—O—C(R₆)—,
—O—C(O)—O—,
—N(R₈)-Q-,
—O—C(R₆)—N(R₈)—,
—C(R₆)—N(OR₉)—,

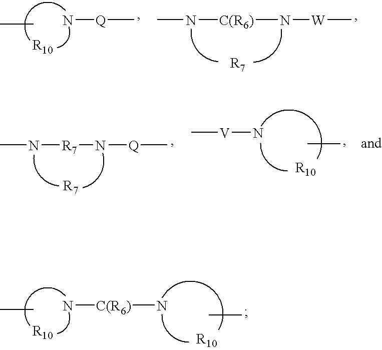

R₄ is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the alkyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups can be unsubstituted or substituted by one or more substituents independently selected from the group consisting of alkyl, alkoxy, hydroxyalkylenyl, halo alkylenyl, halo alkyleneoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, (dialkylamino)alkyleneoxy, and in the case of alkyl and heterocyclyl, oxo;

R₅ is selected from the group consisting of:

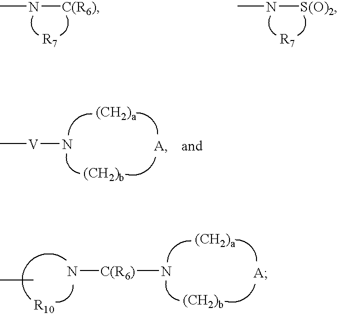

R₆ is selected from the group consisting of ═O and ═S;

R₇ is C₂₋₇ alkylene;

R₈ is selected from the group consisting of hydrogen, alkyl, alkoxyalkylenyl, and arylalkylenyl;

R₉ is selected from the group consisting of hydrogen and alkyl;

R₁₀ is C₃₋₈ alkylene;

A is selected from the group consisting of —O—, —C(O)—, —CH₂—, —S(O)₀₋₂—, and —N(R₄)—;

Q is selected from the group consisting of a bond, —C(R₆)—, —C(R₆)—C(R₆)—, —S(O)₂—, —C(R₆)—N(R₈)—W—, —S(O)₂—N(R₈)—, —C(R₆)—O—, —C(R₆)—S—, and —C(R₆)—N(OR₉)—;

V is selected from the group consisting of —O—C(R₆)— and —N(R₈)—C(R₆)—;

W is selected from the group consisting of a bond, —C(O)—, and —S(O)₂—; and

a and b are independently integers from 1 to 6 with the proviso that a+b is ≧7; or a pharmaceutically acceptable salt thereof.

In one embodiment, the present invention provides an intermediate imidazonaphthyridine compound selected from the group consisting of the following Formulas XIV, XV, XVI, and XVII: 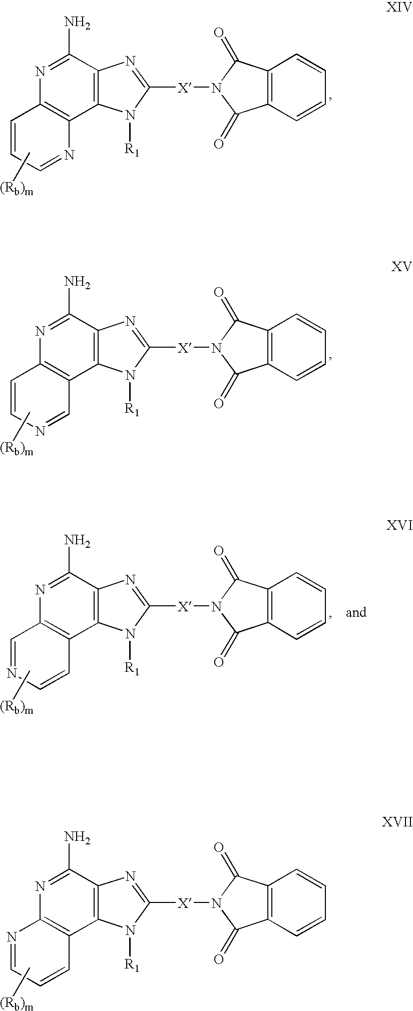
wherein:

X′ is selected from the group consisting of C₁₋₄ alkylene and C₂₋₄ alkenylene;

R_{b} is selected from the group consisting of halogen, hydroxy, alkyl, haloalkyl, alkoxy, and —N(R₉)₂;

m is an integer from 0 to 3;

R₁ is selected from the group consisting of:
—R₄,
—X—R₄,
—X—Y—R₄,
—X—Y—X—Y—R₄, and
—X—R₅;

X is selected from the group consisting of alkylene, arylene, heteroarylene, and heterocyclylene wherein the alkylene group can be optionally interrupted or terminated by arylene, heteroarylene or heterocyclylene and optionally interrupted by one or more —O— groups;

Y is selected from the group consisting of:
—S(O)₀₋₂—,
—C(R₆)—,
—C(R₆)—O—,
—O—C(R₆)—,
—O—C(O)—O—,
—N(R₈)-Q-,
—O—C(R₆)—N(R₈)—,
—C(R₆)—N(OR₉)—,

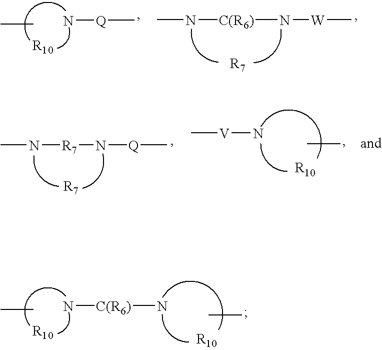

R₄ is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the alkyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups can be unsubstituted or substituted by one or more substituents independently selected from the group consisting of alkyl, alkoxy, hydroxyalkylenyl, haloalkylenyl, haloalkyleneoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, (dialkylamino)alkyleneoxy, and in the case of alkyl and heterocyclyl, oxo;

R₅ is selected from the group consisting of:

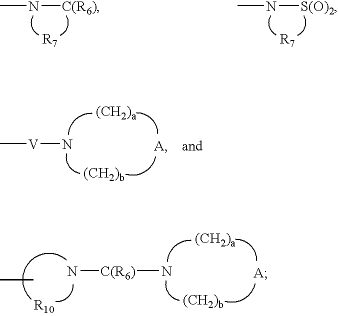

R₆ is selected from the group consisting of ═O and ═S;

R₇ is C₂₋₇ alkylene;

R₈ is selected from the group consisting of hydrogen, alkyl, alkoxyalkylenyl, and arylalkylenyl;

R₉ is selected from the group consisting of hydrogen and alkyl;

R₁₀ is C₃₋₈ alkylene;

A is selected from the group consisting of —O—, —C(O)—, —CH₂—, —S(O)₀₋₂—, and —N(R₄)—;

Q is selected from the group consisting of a bond, —C(R₆)—, —C(R₆)—C(R₆)—, —S(O)₂—, —C(R₆)—N(R₅)—W—, —S(O)₂—N(R₅)—, —C(R₆)—O—, —C(R₆)—S—, and —C(R₆)—N(OR₉)—;

V is selected from the group consisting of —O—C(R₆)— and —N(R₈)—C(R₆)—;

W is selected from the group consisting of a bond, —C(O)—, and —S(O)₂—; and

a and b are independently integers from 1 to 6 with the proviso that a+b is ≦7, or a pharmaceutically acceptable salt thereof.

In one embodiment, the present invention provides a prodrug of the following Formula (XVIII): 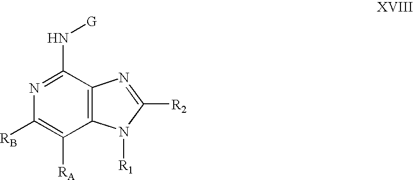
wherein:

R₂ is selected from the group consisting of:
—X′—N(R₈ₐ)—C(R₆)—N(R₈ₐ)—W—R₂₋₁,
—X′—N(R₈ₐ)—C(R₆)—N(OR₈ₐ)—R₂₋₁, 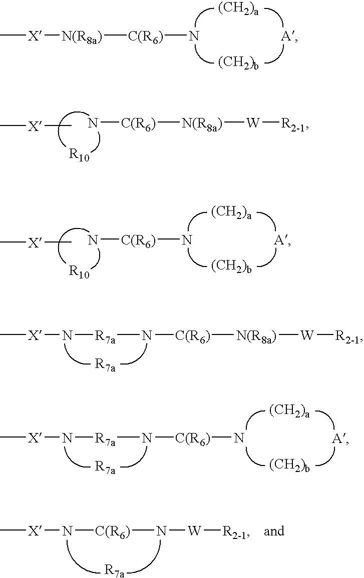
—X′—N(R₈ₐ)—C(R)—O—R₂₋₁;

X′ is selected from the group consisting of C₁₋₄ alkylene and C₂₋₄ alkenylene;

R₂₋₁ is selected from the group consisting of hydrogen, C₁₋₄ alkyl, C₂₋₄ alkenyl, C₂₋₄ alkynyl, aryl, arylC₁₋₄ alkylenyl, aryloxyC₁₋₄ alkylenyl, C₁₋₄ alkylarylenyl, heteroaryl, heteroarylC₁₋₄ alkylenyl, heteroaryloxyC₁₋₄ alkylenyl, C₁₋₄ alkylheteroarylenyl, and heterocyclyl wherein the C₁₋₄ alkyl, C₂₋₄ alkenyl, C₂₋₄ alkynyl, aryl, arylC₁₋₄ alkylenyl, aryloxyC₁₋₄ alkylenyl, C₁₋₄ alkylarylenyl, heteroaryl, heteroarylC₁₋₄ alkylenyl, heteroaryloxyC₁₋₄ alkylenyl, C₁₋₄ alkylheteroarylenyl, and heterocyclyl groups are unsubstituted or substituted by one or more substituents independently selected from the group consisting of C₁₋₄ alkyl, C₁₋₄ alkoxy, C₁₋₄ alkanoyl, C₁₋₄ alkoxycarbonyl, hydroxyC₁₋₄ alkylenyl, haloC₁₋₄ alkylenyl, haloC₁₋₄ alkyleneoxy, halogen, nitro, hydroxy, mercapto, cyano, amino, C₁₋₄ alkylamino, di(C₁₋₄ alkyl)amino, and in the case of C₁₋₄ alkyl, C₂₋₄ alkenyl, C₂₋₄ alkynyl, and heterocyclyl, oxo;

A′ is selected from the group consisting of —O—, —C(O)—, —CH₂—, —S(O)₀₋₂—, —NH—, and —N(C₁₋₄ alkyl)-;

R₇ₐ is C₂₋₄ alkylene;

R₈ₐ is selected from the group consisting of hydrogen and C₁₋₄ alkyl;

G is selected from the group consisting of:
—C(O)—R′,
α-aminoacyl,
α-aminoacyl-α-aminoacyl,
—C(O)—O—R′,
—C(O)—N(R″)R′,
—C(═NY′)—R′,
—CH(OH)—C(O)—OY′,
—CH(OC₁₋₄ alkyl)Y₀,
—CH₂Y₁, and
—CH(CH₃)Y₁;

R′ and R″ are independently selected from the group consisting of C₁₋₁₀ alkyl, C₃₋₇ cycloalkyl, and benzyl, each of which may be unsubstituted or substituted by one or more substitutents selected from the group consisting of halogen, hydroxy, nitro, cyano, carboxy, C₁₋₆ alkyl, C₁₋₄ alkoxy, aryl, heteroaryl, arylC₁₋₄ alkylenyl, heteroarylC₁₋₄ alkylenyl, haloC₁₋₄ alkyl, haloC₁₋₄ alkoxy, —O—C(O)—CH₃, —C(O)—O—CH₃, C(O)—NH₂, —O—CH₂—C(O)—NH₂, —NH₂, and —S(O)₂—NH₂;

α-aminoacyl is an acyl group derived from an amino acid selected from the group consisting of the naturally occurring L-amino acids;

Y′ is selected from the group consisting of hydrogen, C₁₋₆ alkyl, and benzyl;

Y₀ is selected from the group consisting of C₁₋₆ alkyl, carboxyC₁₋₆ alkyl, aminoC₁₋₄ alkyl, mono-N—C₁₋₆ alkylaminoC₁₋₄ alkyl, and di-N,N—C₁₋₆ alkylaminoC₁₋₄ alkyl;
Y₁ is selected from the group consisting of mono-N—C₁₋₆ alkylamino, di-N,N—C₁₋₆ alkylamino, morpholin-4-yl, piperidin-1-yl, pyrrolidin-1-yl, and 4-C₁₋₄ alkylpiperazin-1-yl;

R_{A} and R_{B} are independently selected from the group consisting of:
hydrogen,
halogen,
alkyl,
alkenyl,
alkoxy,
alkylthio, and
—N(R₉)₂;

or R_{A} and R_{B} taken together form either a fused aryl ring that is unsubstituted or substituted by one or more Rₐ groups, or a fused 5 to 7 membered saturated ring that is unsubstituted or substituted by one or more R_{c} groups;

or R_{A} and R_{B} taken together form a fused heteroaryl or 5 to 7 membered saturated ring containing one heteroatom selected from the group consisting of N and S, wherein the heteroaryl ring is unsubstituted or substituted by one or more R_{b} groups, and the 5 to 7 membered saturated ring is unsubstituted or substituted by one or more Rₐ groups;

Rₐ is selected from the group consisting of halogen, alkyl, haloalkyl, alkoxy, and —N(R₉)₂;

R_{b} is selected from the group consisting of halogen, hydroxy, alkyl, haloalkyl, alkoxy, and —N(R₉)₂;

R_{c} is selected from the group consisting of halogen, hydroxy, alkyl, alkenyl, haloalkyl, alkoxy, alkylthio, and —N(R₉)₂;

R₁ is selected from the group consisting of:
—R₄,
X—R₄,
—X—Y—R₄,
—X—Y—X—Y—R₄, and
—X—R₅;

X is selected from the group consisting of alkylene, arylene, heteroarylene, and heterocyclylene wherein the alkylene group can be optionally interrupted or terminated by arylene, heteroarylene or heterocyclylene and optionally interrupted by one or more —O— groups;

Y is selected from the group consisting of:
—S(O)₀₋₂—,
—C(R₆)—,
—C(R₆)—O—,
—O—C(R₆)—,
—O—C(O)—O—,
—N(R₈)-Q-,
—O—C(R₆)—N(R₈)—,
—C(R₆)—N(OR₉)—,

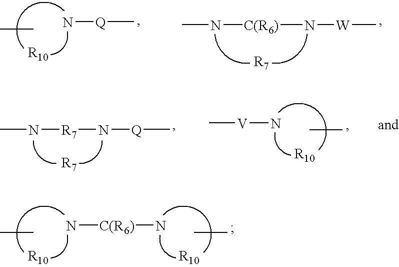

R₄ is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the alkyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups can be unsubstituted or substituted by one or more substituents independently selected from the group consisting of alkyl, alkoxy, hydroxyalkylenyl, haloalkylenyl, haloalkyleneoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, (dialkylamino)alkyleneoxy, and in the case of alkyl and heterocyclyl, oxo;

R₅ is selected from the group consisting of:

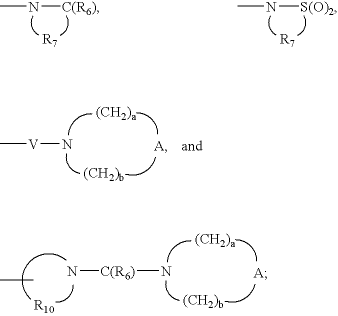

R₆ is selected from the group consisting of ═O and ═S;

R₇ is C₂₋₇ alkylene;

R₈ is selected from the group consisting of hydrogen, alkyl, alkoxyalkylenyl, and arylalkylenyl;

R₉ is selected from the group consisting of hydrogen and alkyl;

R₁₀ is C₃₋₈ alkylene;

A is selected from the group consisting of —O—, —C(O)—, —CH₂—, —S(O)₀₋₂—, and —N(R₄)—;

Q is selected from the group consisting of a bond, —C(R₆)—, —C(R₆)—C(R₆)—, —S(O)₂—, —C(R₆)—N(R₈)—W—, —S(O)₂—N(R₈)—, —C(R₆)—O—, —C(R₆)—S—, and —C(R₆)—N(OR₉)—;

V is selected from the group consisting of —O—C(R₆)— and —N(R₈)—C(R₆)—;

W is selected from the group consisting of a bond, —C(O)—, and —S(O)₂—; and

a and b are independently integers from 1 to 6 with the proviso that a+b is ≦7;

with the proviso that when R_{A} and R_{B} taken together form a ring, and X is interrupted with one —O— group, then Y is other than —S(O)₀₋₂—; and

with the further proviso that when R_{A} and R_{B} are independently hydrogen, halogen, alkyl, alkenyl, alkoxy, alkylthio, or —N(R₉)₂, and R₂ is selected from the group consisting of: 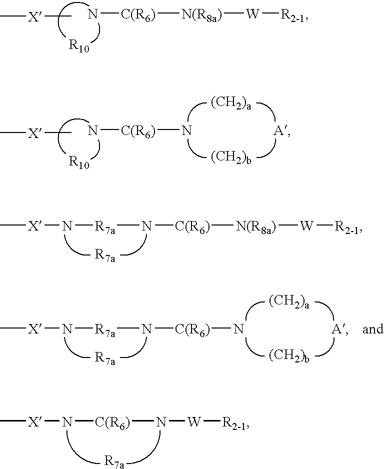
then X is not interrupted with one or more —O— groups and Y is other than —S(O)₀₋₂—;
or a pharmaceutically acceptable salt thereof.

As used herein, the terms “alkyl”, “alkenyl”, “alkynyl”, and the prefix “alk-” are inclusive of both straight chain and branched chain groups and of cyclic groups, e.g. cycloalkyl and cycloalkenyl. Unless otherwise specified, these groups contain from 1 to 20 carbon atoms, with alkenyl groups containing from 2 to 20 carbon atoms, and alkynyl groups containing from 2 to 20 carbon atoms. In some embodiments, these groups have a total of up to 10 carbon atoms, up to 8 carbon atoms, up to 6 carbon atoms, or up to 4 carbon atoms. Cyclic groups can be monocyclic or polycyclic and preferably have from 3 to 10 ring carbon atoms. Exemplary cyclic groups include cyclopropyl, cyclopropylmethyl, cyclopentyl, cyclohexyl, adamantyl, and substituted and unsubstituted bornyl, norbornyl, and norbornenyl.

Unless otherwise specified, “alkylene”, “alkenylene”, and “alkynylene” are the divalent forms of the “alkyl”, “alkenyl”, and “alkynyl” groups defined above. The terms, “alkylenyl”, “alkenylenyl”, and “alkynylenyl” are used when “alkylene”, “alkenylene”, and “alkynylene”, respectively, are substituted. For example, an arylalkylenyl group comprises an alkylene moiety to which an aryl group is attached.

The term “haloalkyl” is inclusive of groups that are substituted by one or more halogen atoms, including perfluorinated groups. This is also true of other groups that include the prefix “halo-”. Examples of suitable haloalkyl groups are chloromethyl, trifluoromethyl, and the like.

The term “aryl” as used herein includes carbocyclic aromatic rings or ring systems. Examples of aryl groups include phenyl, naphthyl, biphenyl, fluorenyl and indenyl.

Unless otherwise indicated, the term “heteroatom” refers to the atoms O, S, or N.

The term “heteroaryl” includes aromatic rings or ring systems that contain at least one ring heteroatom (e.g., O, S, N). In some embodiments, the term “heteroaryl” includes a ring or ring system that contains 2 to 12 carbon atoms, 1 to 3 rings, 1 to 4 heteroatoms, and O, S, and/or N as the heteroatoms. Suitable heteroaryl groups include furyl, thienyl, pyridyl, quinolinyl, isoquinolinyl, indolyl, isoindolyl, triazolyl, pyrrolyl, tetrazolyl, imidazolyl, pyrazolyl, oxazolyl, thiazolyl, benzofuranyl, benzothiophenyl, carbazolyl, benzoxazolyl, pyrimidinyl, benzimidazolyl, quinoxalinyl, benzothiazolyl, naphthyridinyl, isoxazolyl, isothiazolyl, purinyl, quinazolinyl, pyrazinyl, 1-oxidopyridyl, pyridazinyl, triazinyl, tetrazinyl, oxadiazolyl, thiadiazolyl, and so on.

The term “heterocyclyl” includes non-aromatic rings or ring systems that contain at least one ring heteroatom (e.g., O, S, N) and includes all of the fully saturated and partially unsaturated derivatives of the above mentioned heteroaryl groups. In some embodiments, the term “heterocyclyl” includes a ring or ring system that contains 2 to 12 carbon atoms, 1 to 3 rings, 1 to 4 heteroatoms, and O, S, and N as the heteroatoms. Exemplary heterocyclyl groups include pyrrolidinyl, tetrahydrofuranyl, morpholinyl, thiomorpholinyl, 1,1-dioxothiomorpholinyl, piperidinyl, piperazinyl, thiazolidinyl, imidazolidinyl, isothiazolidinyl, tetrahydropyranyl, quinuclidinyl, homopiperidinyl(azepanyl), 1,4-oxazepanyl, homopiperazinyl(diazepanyl), 1,3-dioxolanyl, aziridinyl, azetidinyl, dihydroisoquinolin-(1H)-yl, octahydroisoquinolin-(1H)-yl, dihydroquinolin-(2H)-yl, octahydroquinolin-(2H)-yl, dihydro-1H-imidazolyl, 3-azabicyclo[3.2.2]non-3-yl, and the like.

The term “heterocyclyl” includes bicylic and tricyclic heterocyclic ring systems. Such ring systems include fused and/or bridged rings and spiro rings. Fused rings can include, in addition to a saturated or partially saturated ring, an aromatic ring, for example, a benzene ring. Spiro rings include two rings joined by one spiro atom and three rings joined by two spiro atoms.

When “heterocyclyl” contains a nitrogen atom, the point of attachment of the heterocyclyl group may be the nitrogen atom.

The terms “arylene”, “heteroarylene”, and “heterocyclylene” are the divalent forms of the “aryl”, “heteroaryl”, and “heterocyclyl” groups defined above. The terms, “arylenyl”, “heteroarylenyl”, and “heterocyclylenyl” are used when “arylene”, “heteroarylene”, and “heterocyclylene”, respectively, are substituted. For example, an alkylarylenyl group comprises an arylene moiety to which an alkyl group is attached.

The term “fused aryl ring” includes fused carbocyclic aromatic rings or ring systems. Examples of fused aryl rings include benzo, naphtho, fluoreno, and indeno.

The term “fused heteroaryl ring” includes the fused forms of 5 or 6 membered aromatic rings that contain one heteroatom selected from S and N.

The term “fused 5 to 7 membered saturated ring” includes rings which are fully saturated except for the bond where the ring is fused.

When a group (or substituent or variable) is present more than once in any formula described herein, each group (or substituent or variable) is independently selected, whether explicitly stated or not. For example, for the formula —N(R₉)₂— each R₉ group is independently selected. In another example, when an R₁ and an R₂ group both contain an R₁₀ group, each R₁₀ group is independently selected.

The invention is inclusive of the compounds described herein and salts thereof, in any of their pharmaceutically acceptable forms, including isomers (e.g., diastereomers and enantiomers), solvates, polymorphs, prodrugs, and the like. In particular, if a compound is optically active, the invention specifically includes each of the compound's enantiomers as well as racemic mixtures of the enantiomers. It should be understood that the term “compound” or the term “compounds” includes any or all of such forms, whether explicitly stated or not (although at times, “salts” are explicitly stated).

The term “prodrug” means a compound that can be transformed in vivo to yield an immune response modifying compound in any of the salt, solvated, polymorphic, or isomeric forms described above. The prodrug, itself, may be an immune response modifying compound in any of the salt, solvated, polymorphic, or isomeric forms described above. The transformation may occur by various mechanisms, such as through a chemical (e.g., solvolysis or hydrolysis, for example, in the blood) or enzymatic biotransformation. A discussion of the use of prodrugs is provided by T. Higuchi and W. Stella, “Pro-drugs as Novel Delivery Systems,” Vol. 14 of the A. C. S. Symposium Series, and in Bioreversible Carriers in Drug Design, ed. Edward B. Roche, American Pharmaceutical Association and Pergamon Press, 1987.

For any of the compounds presented herein, each one of the following variables (e.g., R_{A}, R_{B}, R_{A1}, R_{B1}, R_{A2}, R_{B2}, R₁, R₂, m, n, A, G, Q, X, Z, and so on) in any of its embodiments can be combined with any one or more of the other variables in any of their embodiments and associated with any one of the formulas described herein, as would be understood by one of skill in the art. Each of the resulting combinations of variables is an embodiment of the present invention.

In some embodiments, R_{A} and R_{B} are independently selected from the group consisting of hydrogen, halogen, alkyl, alkenyl, alkoxy, alkylthio, and —N(R₉)₂; or R_{A} and R_{B} taken together form either a fused aryl ring that is unsubstituted or substituted by one or more Rₐ groups, or a fused 5 to 7 membered saturated ring that is unsubstituted or substituted by one or more R_{c} groups; or R_{A} and R_{B} taken together form a fused heteroaryl or 5 to 7 membered saturated ring containing one heteroatom selected from the group consisting of N and S, wherein the heteroaryl ring is unsubstituted or substituted by one or more R_{b} groups, and the 5 to 7 membered saturated ring is unsubstituted or substituted by one or more R_{c}, groups.

In certain embodiments (e.g., of Formula I), R_{A} and R_{B} are each independently selected from the group consisting of hydrogen, halogen, alkyl, alkenyl, alkoxy, alkylthio, and —N(R₉)₂.

In certain embodiments (e.g., of Formula I), R_{A} and R_{B} form a fused aryl ring. In certain embodiments, the fused aryl ring is benzo.

In certain embodiments (e.g., of Formula I), R_{A} and R_{B} form a fused heteroaryl ring. In certain embodiments, the fused heteroaryl ring is pyrido or thieno. In certain embodiments, the fused heteroaryl ring is pyrido. In certain of these embodiments, the pyrido ring is 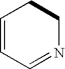
wherein the highlighted bond indicates the position where the ring is fused.

In certain embodiments (e.g., of Formula I), R_{A} and R_{B} form a fused 5 to 7 membered saturated ring. In certain embodiments, the ring is a cyclohexene ring.

In certain embodiments (e.g., of Formula I), R_{A} and R_{B} form a fused 5 to 7 membered saturated ring containing one heteroatom selected from the group consisting of N and S. In certain embodiments the heteroatom is N. In certain embodiments, the ring is tetrahydropyrido or dihydrothieno. In certain embodiments, the ring is tetrahydropyrido. In certain of these embodiments, the ring is 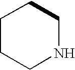
wherein the highlighted bond indicates the position where the ring is fused.

In some embodiments, particularly embodiments of Formula I, the ring formed by R_{A} and R_{B} is unsubstituted.

In certain embodiments, R_{A1} and R_{B1} are independently selected from the group consisting of hydrogen, halogen, alkyl, alkenyl, alkoxy, alkylthio, and —N(R₉)₂. In some embodiments, particularly embodiments of Formula II, R_{A1} and R_{B1} are methyl.

In certain embodiments, R_{A2} and R_{B2} taken together form either a fused aryl ring that is unsubstituted or substituted by one or more Rₐ groups, or a fused 5 to 7 membered saturated ring that is unsubstituted or substituted by one or more R_{c} groups; or R_{A2} and R_{B2} taken together form a fused heteroaryl or 5 to 7 membered saturated ring containing one heteroatom selected from the group consisting of N and S, wherein the heteroaryl ring is unsubstituted or substituted by one or more R_{b} groups, and the 5 to 7 membered saturated ring is unsubstituted or substituted by one or more R_{c} groups.

In certain embodiments, R′ and R″ are independently selected from the group consisting of C₁₋₁₀ alkyl, C₃₋₇ cycloalkyl, and benzyl, each of which may be unsubstituted or substituted by one or more substitutents selected from the group consisting of halogen, hydroxy, nitro, cyano, carboxy, C₁₋₆ alkyl, C₁₋₄ alkoxy, aryl, heteroaryl, arylC₁₋₄ alkylenyl, heteroarylC₁₋₄ alkylenyl, haloC₁₋₄ alkyl, haloC₁₋₄ alkoxy, —O—C(O)—CH₃, —C(O)—O—CH₃, —C(O)—NH₂, —O—CH₂—C(O)—NH₂, —NH₂, and —S(O)₂—NH₂.

In certain embodiments, Rₐ is selected from the group consisting of halogen, alkyl, haloalkyl, alkoxy, and —N(R₉)₂.

In certain embodiments, R_{b} is selected from the group consisting of halogen, hydroxy, alkyl, haloalkyl, alkoxy, and —N(R₉)₂.

In certain embodiments, R_{c} is selected from the group consisting of halogen, hydroxy, alkyl, alkenyl, haloalkyl, alkoxy, alkylthio, and —N(R₉)₂.

In some embodiments, R₁ is selected from the group consisting of —R₄, —X—R₄, —X—Y—R₄, —X—Y—X—Y—R₄, and —X—R₅. In certain embodiments, R₁ is selected from the group consisting of alkyl; arylalkylenyl; heterocyclylalkylenyl that is unsubstituted or substituted by hydroxy, dialkylamino, alkyl, hydroxyalkyl, or heterocyclyl; aryloxyalkylenyl that is unsubstituted or substituted by alkoxy or halogen; hydroxyalkylenyl; aminoalkylenyl; haloalkylenyl; alkylsulfonylalkylenyl; —X—Y—R₄; and —X—R₅. In certain of these embodiments, X is alkylene optionally terminated by heterocyclylene; Y is —N(R₈)-Q-, —C(O)—N(HI)—, 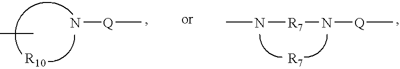
wherein Q is a bond, —C(O)—, —S(O)₂—, —C(O)—N(R₉)—, —C(O)—N(R₈)—C(O)—, —C(S)—N(R₉)—, —C(O)—O—, —C(O)—S—, or —S(O)₂—N(R₅)—; R₄ is hydrogen, alkyl, arylalkylenyl, heterocyclylalkylenyl, arylalkenylenyl, aryl, heteroaryl, or heterocyclyl, wherein aryl, heteroaryl, and heterocyclyl are unsubstituted or substituted by one or more substituents independently selected from the group consisting of alkyl, halogen, cyano, alkoxy, aryl, and haloalkyl; and R₅ is 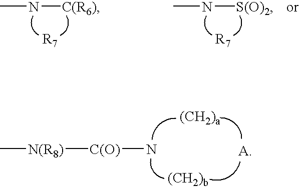
In some embodiments, particularly embodiments of Formula II, R₁ is selected from the group consisting of alkyl, arylalkylenyl, heterocyclylalkylenyl, aryloxyalkylenyl, hydroxyalkylenyl, aminoalkylenyl, haloalkylenyl, alkylsulfonylalkylenyl, —X—Y—R₄, and —X—R₅. In certain of these embodiments, X is alkylene; Y is —N(R₈)—C(O)—, —N(R₈)—S(O)₂—, —N(R₅)—C(O)—N(R₈)—, —N(R₈)—C(S)—N(R₈)—, —N(R₈)—S(O)₂—N(R₈)—, or 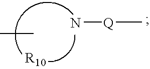
R₄ is alkyl, aryl, or heteroaryl; and R₅ is 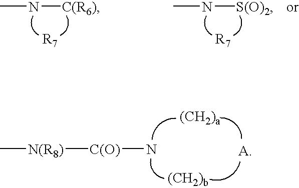
In some embodiments, particularly embodiments of Formulas I, III, IV, V, VI, VII, and VIII, R₁ is selected from the group consisting of alkyl, arylalkylenyl, aryloxyalkylenyl, hydroxyalkylenyl, aminoalkylenyl, haloalkylenyl, alkylsulfonylalkylenyl, —X—Y—R₄, and —X—R₅. In certain of these embodiments, X is alkylene; Y is —N(R₈)—C(O)—, —N(R₈)—S(O)₂—, —N(R₈)—C(O)—N(R₈)—, —N(R₈)—C(S)—N(R₈)—, or —N(R₈)—S(O)₂—N(R₈)—; R₄ is alkyl, aryl, or heteroaryl; and R₅ is 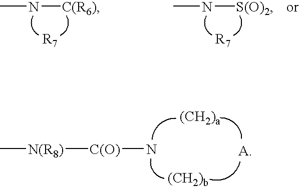
In certain embodiments R₁ is alkyl or hydroxyalkylenyl. In certain embodiments, particularly embodiments of Formulas X, XI, XII, XIII, XIV, XV, XVI, and XVII, R₁ is selected from the group consisting of C₁₋₁₀ alkyl, hydroxyC₁₋₆ alkylenyl, C₁₋₄ alkyl-O—C₁₋₆ alkylenyl, phenyl-C₁₋₄ alkylenyl, and phenyl; wherein phenyl is unsubstituted or substituted with one or two substituents selected from the group consisting of C₁₋₄ alkyl, C₁₋₄ alkoxy, and halogen. In some embodiments, particularly embodiments of Formulas X, XI, XII, XIII, XIV, XV, XVI, and XVII, R₁ is C₁₋₁₀ alkyl or hydroxyC₁₋₆ alkylenyl.

In certain embodiments, R₁ is selected from the group consisting of 2-methylpropyl, 2-hydroxy-2-methylpropyl, 2-methyl-2-[(methylsulfonyl)amino]propyl, 4-[(methylsulfonyl)amino]butyl, 4-[(morpholin-4-ylcarbonyl)amino]butyl, (1-hydroxycyclohexyl)methyl, (1-hydroxycyclobutyl)methyl, and tetrahydro-2H-pyran-4-ylmethyl.

In certain embodiments, R₂ is selected from the group consisting of —X′—N(R₈ₐ)—C(R₆)—N(R₈ₐ)—W—R₂₋₁, —X′—N(R₈ₐ)—C(R₆)—N(OR₈ₐ)—R₂₁ᵢ, 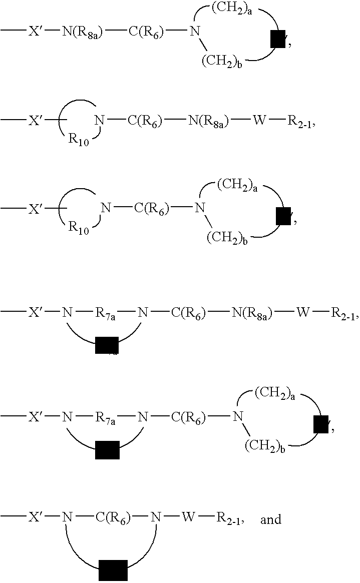
—X′—N(R₈ₐ)—C(R₆)—O—R₂₋₁.

In some embodiments of Formulas I, II, III, IV, V, VI, VII, and VIII, R₂ is selected from the group consisting of —X′—N(R₈ₐ)—C(R₆)—N(R₈ₐ)—W—R₂₋₁, —X′—N(R₈ₐ)—C(R₆)—N(OR₈ₐ)—R₂₋₁,

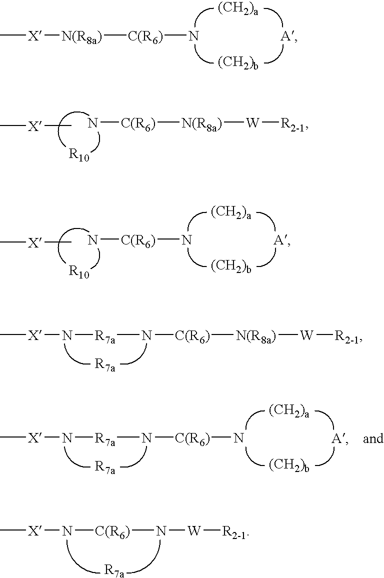

In certain embodiments, particularly embodiments of Formula III, IV, V, VI, VII, and VIII, R₂ is selected from the group consisting of —X′—N(R₈ₐ)—C(R₆)—N(R₈ₐ)—W—R₂₋₁, —X′—N(R₈ₐ)—C(R₆)—N(OR₈ₐ)—R₂₋₁,

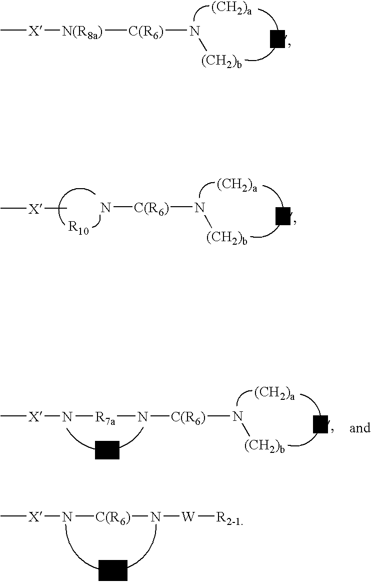

In some embodiments, particularly embodiments of Formulas I and II, R₂ is selected from the group consisting of —X′—N(Rₐ)—C(R₆)—N(R₈ₐ)—W—R₂₋₁, —X′—N(R₈ₐ)—C(R₆)—N(OR₈ₐ)—R₂₋₁,

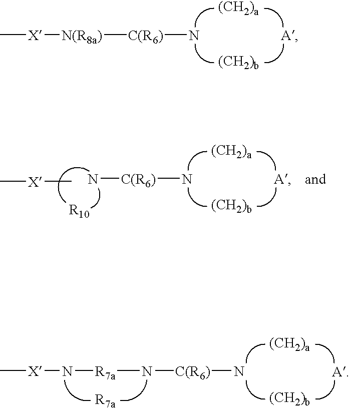

In some embodiments of Formulas I, II, III, IV, V, VI, VII, and VIII, R₂ is selected from the group consisting of —X′—N(R₈ₐ)—C(R₆)—N(R₈ₐ)—W—R₂₋₁, —X′—N(R₈ₐ)—C(R^{r})—N(OR₈ₐ)—R₂₋₁, and

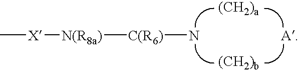

In certain embodiments of Formulas I, II, III, IV, V, VI, VII, and VIII, R₂ is —X′—N(R₈ₐ)—C(R₆)—N(R₈ₐ)—W—R₂₋₁.

In certain embodiments of Formulas I, II, III, IV, V, VI, VII, and VIII, R₂ is —X′—N(R₈ₐ)—C(R₆)—N(OR₈ₐ)—R₂₋₁.

In certain embodiments of Formulas I, II, III, IV, V, VI, VII, and VIII, R₂ is

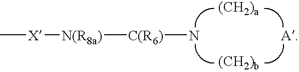

In certain embodiments, R₂ is —X′—N(R₈ₐ)—C(R₆)—O—R₂₋₁.

In certain embodiments, R₂₋₁ is selected from the group consisting of hydrogen, C₁₋₄ alkyl, C₂₋₄ alkenyl, C₂₋₄ alkynyl, aryl, arylC₁₋₄ alkylenyl, aryloxyC₁₋₄ alkylenyl, C₁₋₄ alkylarylenyl, heteroaryl, heteroarylC₁₋₄ alkylenyl, heteroaryloxyC₁₋₄ alkylenyl, C₁₋₄ alkylheteroarylenyl, and heterocyclyl wherein the C₁₋₄ alkyl, C₂₋₄ alkenyl, C₂₋₄ alkynyl, aryl, arylC₁₋₄ alkylenyl, aryloxyC₁₋₄ alkylenyl, C₁₋₄ alkylarylenyl, heteroaryl, heteroarylC₁₋₄ alkylenyl, heteroaryloxyC₁₋₄ alkylenyl, C₁₋₄ alkylheteroarylenyl, and heterocyclyl groups are unsubstituted or substituted by one or more substituents independently selected from the group consisting of C₁₋₄ alkyl, C₁₋₄ alkoxy, C₁₋₄ alkanoyl, C₁₋₄ alkoxycarbonyl, hydroxyC₁₋₄ alkylenyl, haloC₁₋₄ alkylenyl, haloC₁₋₄ alkyleneoxy, halogen, nitro, hydroxy, mercapto, cyano, amino, C₁₋₄ alkylamino, di(C₁₋₄ alkyl)amino, and in the case of C₁₋₄ alkyl, C₂₋₄ alkenyl, C₂₋₄ alkynyl, and heterocyclyl, oxo.

In certain embodiments, R₂₋₁ is selected from the group consisting of hydrogen,

C₁₋₄ alkyl, aryl, heteroaryl, arylC₁₋₄ alkylenyl, substituted aryl wherein the substituent is C₁₋₄ alkyl, C₁₋₄ alkoxy, C₁₋₄ alkanoyl, cyano, di(C₁₋₄ alkyl)amino, haloC₁₋₄ alkylenyl, nitro, or halogen, or substituted C₄ alkyl wherein the substituent is C₁₋₄ alkoxycarbonyl or di(C₁₋₄ alkyl)amino. In certain of these embodiments, R₂₋₁ is selected from the group consisting of hydrogen, methyl, and ethyl.

In some embodiments of Formulas I, II, III, IV, V, VI, VII, and VIII, R₂₋₁ is selected from the group consisting of C₁₋₄ alkyl, aryl, or substituted aryl wherein the substituent is C₁₋₄ alkyl, C₁₋₄ alkoxy, or halogen. In certain of these embodiments, R₂₋₁ is selected from the group consisting of C₁₋₄ alkyl, phenyl, or substituted phenyl wherein the substituent is C₁₋₄ alkyl, C₁₋₄ alkoxy, or halogen. In certain of these embodiments, R₂₋₁ is selected from the group consisting of methyl and ethyl.

In certain embodiments, R₄ is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the alkyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups can be unsubstituted or substituted by one or more substituents independently selected from the group consisting of alkyl, alkoxy, hydroxyalkylenyl, haloalkylenyl, haloalkyleneoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, (dialkylamino)alkyleneoxy, and in the case of alkyl and heterocyclyl, oxo. In certain embodiments, R₄ is hydrogen, alkyl, arylalkylenyl, heterocyclylalkylenyl, arylalkenylenyl, aryl, heteroaryl, or heterocyclyl, wherein aryl, heteroaryl, and heterocyclyl are unsubstituted or substituted by one or more substituents independently selected from the group consisting of alkyl, halogen, cyano, alkoxy, aryl, and haloalkyl. In certain embodiments, R₄ is alkyl, aryl, or heteroaryl.

In certain embodiments, R₅ is selected from the group consisting of 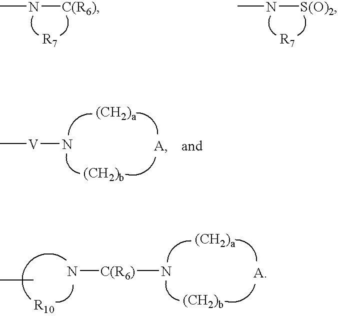
In certain embodiments, R₅ is 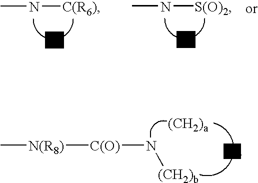
In certain embodiments, R₅ is

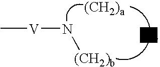

In certain embodiments, R₆ is selected from the group consisting of ═O and ═S. In certain embodiments, R₆ is ═O. In certain embodiments, R₆ is ═S.

In certain embodiments, R₇ is C₂₋₇ alkylene. In certain embodiments, R₇ is C₂₋₄ alkylene. In certain embodiments, R₇ is ethylene.

In certain embodiments, R₇ₐ is C₂₋₄ alkylene. In certain embodiments, R₇ₐ is C₂₋₃ alkylene. In certain embodiments, R₇ₐ is ethylene.

In certain embodiments, R₈ is selected from the group consisting of hydrogen, alkyl, alkoxyalkylenyl, and arylalkylenyl.

In certain embodiments, R₈ₐ is selected from the group consisting of hydrogen and C₁₋₄ alkyl. In certain embodiments, R₈ₐ is hydrogen. In certain embodiments, R₈ₐ is methyl.

In certain embodiments, R₉ is selected from the group consisting of hydrogen and alkyl.

In certain embodiments, R₁₀ is C₃₋₈ alkylene. In certain embodiments, R₁₀ is C₃₋₆ alkylene. In certain embodiments, R₁₀ is pentylene.

In certain embodiments, A is selected from the group consisting of —O—, —C(O)—, —CH₂—, —S(O)₀₋₂—, and —N(R₄)—. In certain embodiments, A is —O—.

In certain embodiments, A′ is selected from the group consisting of —O—, —C(O)—, —CH₂—, —S(O)₀₋₂—, —NH—, and —N(C₁₋₄ alkyl)-. In certain embodiments, A′ is —O—.

In certain embodiments, G is selected from the group consisting of —C(O)—R′, α-aminoacyl, α-aminoacyl-α-aminoacyl, —C(O)—O—R′, —C(O)—N(R″)R′, —C(═NY′)—R′, —CH(OH)—C(O)—OY′, —CH(OC₁₋₄ alkyl)Y₀, —CH₂Y₁, and —CH(CH₃)Y₁, wherein α-aminoacyl is an acyl group derived from an amino acid selected from the group consisting of the naturally occurring L-amino acids.

In certain embodiments, Q is selected from the group consisting of a bond, —C(R₆)—, —C(R₆)—C(R₆)—, —S(O)₂—, —C(R₆)—N(R₈)—W—, —S(O)₂—N(R₈)—, —C(R₆)—O—, —C(R₆)—S—, and —C(R₆)—N(OR₉)—. In certain embodiments, Q is selected from the group consisting of a bond, —C(R₆)—, —C(R₆)—C(R₆)—, —S(O)₂—, —C(R₆)—N(R₈)—W—, —S(O)₂—N(R₈)—, —C(R₆)—O—, and —C(R₆)—N(OR₉)—. In certain embodiments, Q is selected from the group consisting of a bond, —C(O)—, —S(O)₂—, —C(O)—N(R₈)—, —C(O)—N(R₅)—C(O)—, —C(S)—N(R₅)—, —C(O)—O—, —C(O)—S—, and —S(O)₂—N(R₈)—.

In certain embodiments, V is selected from the group consisting of —O—C(R₆)— and —N(R₈)—C(R₆)—. In certain embodiments, V is —N(R₉)—C(O)—.

In certain embodiments, W is selected from the group consisting of a bond, —C(O)—, and —S(O)₂—. In some embodiments, particularly embodiments of Formulas I, II, III, IV, V, VI, VII, and VIII, W is a bond.

In certain embodiments, X is selected from the group consisting of alkylene, arylene, heteroarylene, and heterocyclylene wherein the alkylene group can be optionally interrupted or terminated by arylene, heteroarylene or heterocyclylene and optionally interrupted by one or more —O— groups. In some embodiments, particularly embodiments of Formulas I, III, IV, V, VI, VII, VIII, X, XI, XII, XIII, XIV, XV, XVI, and XVII, and more particularly embodiments of Formula II, X is selected from the group consisting of alkylene, arylene, heteroarylene, and heterocyclylene wherein the alkylene group can be optionally interrupted or terminated by arylene, heteroarylene or heterocyclylene.

In some embodiments, X is not interrupted with an —O— group. For example, particularly in embodiments of Formula I, when R_{A} and R_{B} are independently hydrogen, halogen, alkyl, alkenyl, alkoxy, alkylthio, or —N(R₉)₂, then X is not interrupted with an —O— group. In some embodiments, when R_{A} and R_{B} are independently hydrogen, halogen, alkyl, alkenyl, alkoxy, alkylthio, or —N(R₉)₂, and R₂ is selected from the group consisting of 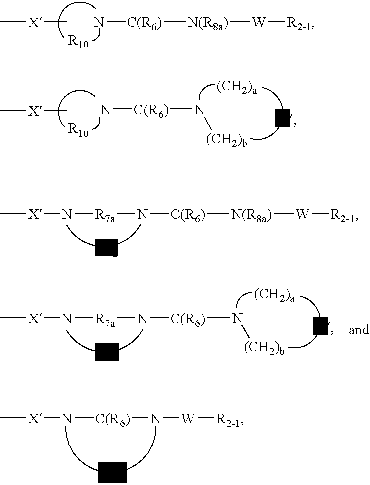
then X is not interrupted with one or more —O— groups. In some embodiments, when R₂ is selected from the group consisting of 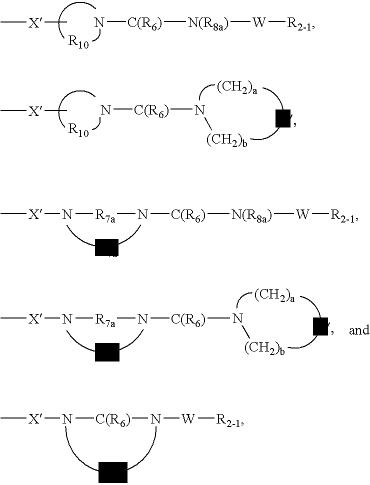
then X is not interrupted with one or more —O— groups.

In certain embodiments, X is alkylene. In certain embodiments, X is alkylene optionally terminated by heterocyclylene.

In certain embodiments, X′ is selected from the group consisting of C₁₋₄ alkylene and C₂₋₄ alkenylene. In some embodiments, particularly embodiments of Formulas I, II, III, IV, V, VI, VII, and VIII, X′ is C₁₋₄ alkylene, and in certain embodiments X′ is methylene or ethylene. In certain embodiments, X′ is methylene.

In certain embodiments, Y is selected from the group consisting of —S(O)₀₋₂—, —C(R₆)—, —C(R₆)—O—, —O—C(R₆)—, —O—C(O)—O—, —N(R₈)-Q-, —O—C(R₆)—N(R₈)—, —C(R₆)—N(OR₉)—, 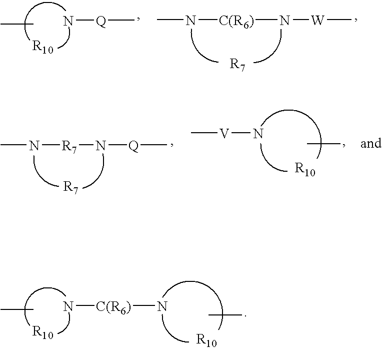
In certain embodiments, Y is selected from the group consisting of —S(O)₀₋₂—, —C(R₆)—, —C(R₆)—O—, —O—C(R₆)—, —O—C(O)—O—, —N(R₈)-Q-, —O—C(R₆)—N(R₈)—, —C(R₆)—N(OR₉)—, and

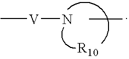

In certain embodiments, Y is —N(R₈)-Q-, —C(O)—N(H)—, 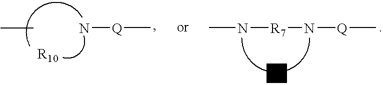
In certain embodiments, Y is —N(R₈)—C(O)—, —N(R₈)—S(O)₂—, —N(R₅)—C(O)—N(R₅)—, —N(R₅)—C(S)—N(R₅)—, —N(R₈)—S(O)₂—N(R₈)—, or 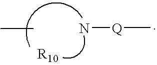
In certain embodiments, Y is —N(R₈)—C(O)—, —N(R₈)—S(O)₂—, —N(R₈)—C(O)—N(R₈)—, —N(R₈)—C(S)—N(R₈)—, or —N(R₉)—S(O)₂—N(R₈)—.

In certain embodiments, Y is other than —S(O)₀₋₂—. For example, particularly in embodiments of Formula I, when X is interrupted with one —O— group, then Y is other than —S(O)₀₋₂—. In some embodiments, when R_{A} and R_{B} taken together form a ring, and X is interrupted with one —O— group, then Y is other than —S(O)₀₋₂—. In some embodiments, when R_{A} and R_{B} are independently hydrogen, halogen, alkyl, alkenyl, alkoxy, alkylthio, or —N(R₉)₂, and R₂ is selected from the group consisting of 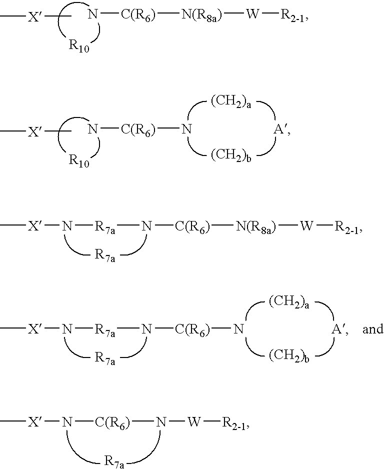
Y is other than —S(O)₀₋₂—. In some embodiments, when R₂ is selected from the group consisting of 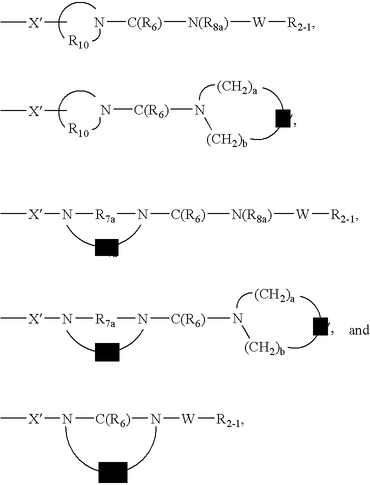
Y is other than —S(O)₀₋₂—.

In certain embodiments, Y′ is selected from the group consisting of hydrogen, C₁₋₆ alkyl, and benzyl.

In certain embodiments, Y₀ is selected from the group consisting of C₁₋₆ alkyl, carboxyC₁₋₆ alkyl, aminoC₁₋₄ alkyl, mono-N—C₁₋₆ alkylaminoC₁₋₄ alkyl, and di-N,N—C₁₋₆ alkylaminoC₁₋₄ alkyl.

In certain embodiments, Y₁ is selected from the group consisting of mono-N—C₁₋₆ alkylamino, di-N,N—C₁₋₆ alkylamino, morpholin-4-yl, piperidin-1-yl, pyrrolidin-1-yl, and 4-C₁₋₄ alkylpiperazin-1-yl.

In some embodiments, a and b are independently integers from 1 to 6 with the proviso that a+b is ≦7. In some embodiments, a and b are independently integers from 1 to 4 with the proviso that a+b is ≦5.

In some embodiments, n is an integer from 0 to 4. In some embodiments, particularly embodiments of Formulas III and IV, n is 0.

In some embodiments, m is an integer from 0 to 3. In some embodiments, particularly embodiments of Formula V, VI, VII, and VIII, m is 0.

In some embodiments, the imidazonaphthyridine compounds are of the following formula (V): 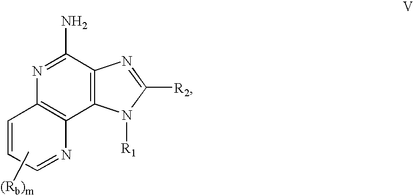
or a pharmaceutically acceptable salt thereof.

In some embodiments the intermediate imidazonaphthyridine compounds are of the following formula (XIV): 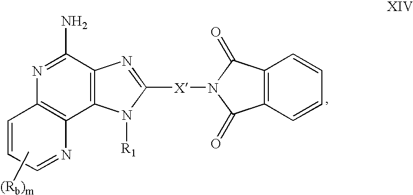
or a pharmaceutically acceptable salt thereof.

In certain embodiments, R₁ is selected from the group consisting of alkyl; arylalkylenyl; heterocyclylalkylenyl that is unsubstituted or substituted by hydroxy, dialkylamino, alkyl, hydroxyalkyl, or heterocyclyl; aryloxyalkylenyl that is unsubstituted or substituted by alkoxy or halogen; hydroxyalkylenyl; aminoalkylenyl; haloalkylenyl; alkylsulfonylalkylenyl; —X—Y—R₄; and —X—R₅; wherein X is alkylene optionally terminated by heterocyclylene; Y is —N(R₈)-Q-, —C(O)—N(H)—, 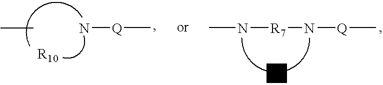
wherein Q is a bond, —C(O)—, —S(O)₂—, —C(O)—N(R₈)—, —C(O)—N(R₈)—C(O)—, —C(S)—N(R₈)—, —C(O)—O—, —C(O)—S—, or —S(O)₂—N(R₈)—; R₄ is hydrogen, alkyl, arylalkylenyl, heterocyclylalkylenyl, arylalkenylenyl, aryl, heteroaryl, or heterocyclyl, wherein aryl, heteroaryl, and heterocyclyl are unsubstituted or substituted by one or more substituents independently selected from the group consisting of alkyl, halogen, cyano, alkoxy, aryl, and haloalkyl; and R₅ is

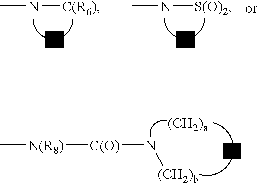

In certain embodiments, R₁ is selected from the group consisting of alkyl, arylalkylenyl, heterocyclylalkylenyl, aryloxyalkylenyl, hydroxyalkylenyl, aminoalkylenyl, haloalkylenyl, alkylsulfonylalkylenyl, —X—Y—R₄, and —X—R₅; wherein X is alkylene; Y is —N(R₈)—C(O)—, —N(R₈)—S(O)₂—, —N(R₈)—C(O)—N(R)—, —N(R₈)—C(S)—N(R₈)—, —N(R₈)—S(O)₂—N(R₈)—, or 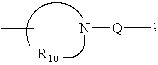
R₄ is alkyl, aryl, or heteroaryl; and R₅ is

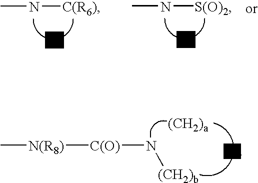

In certain embodiments, R₁ is selected from the group consisting of alkyl, arylalkylenyl, aryloxyalkylenyl, hydroxyalkylenyl, aminoalkylenyl, haloalkylenyl, alkylsulfonylalkylenyl, —X—Y—R₄, and —X—R₅; wherein X is alkylene; Y is —N(R₈)—C(O)—, —N(R₈)—S(O)₂—, —N(R₈)—C(O)—N(R₅)—, —N(R₈)—C(S)—N(R₈)—, or —N(R₈)—S(O)₂—N(R₈)—; 4 is alkyl, aryl, or heteroaryl; and R₅ is

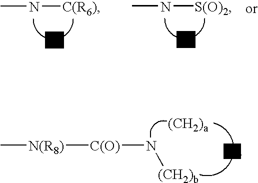

In certain embodiments, W is a bond, and R₂₋₁ is selected from the group consisting of hydrogen, methyl, and ethyl.

In some embodiments of Formulas I, II, III, IV, V, VI, VII, and VIII, W is a bond, and R₂₋₁ is selected from the group consisting of C₁₋₄ alkyl, phenyl, or substituted phenyl wherein the substituent is C₁₋₄ alkyl C₁₋₄ alkoxy, or halogen.

In some embodiments of Formulas I, II, III, IV, V, VI, VII, and VIII, R₂ is —X′—N(R₈ₐ)—C(R₆)—N(R₈ₐ)—W—R₂₋₁ and R₂₋₁ is selected from the group consisting of C₁₋₄ alkyl, aryl, or substituted aryl wherein the substituent is C₁₋₄ alkyl, C₁₋₄ alkoxy, or halogen.

In some embodiments of Formulas I, II, III, IV, V, VI, VII, and VIII, R₂ is —X′—N(R₈ₐ)—C(R₆)—N(R₈ₐ)—W—R₂₋₁, W is a bond, and R₂₋₁ is selected from the group consisting of C₁₋₄ alkyl, aryl, or substituted aryl wherein the substituent is C₁₋₄ alkyl, C₁₋₄ alkoxy, or halogen.

In some embodiments, R₂ is selected from the group consisting of —X′—N(R₈ₐ)—C(R₆)—N(R₈ₐ)—W—R₂₁, X′—N(R₈ₐ)—C(R₆)—N(OR₈ₐ)—R₂—, 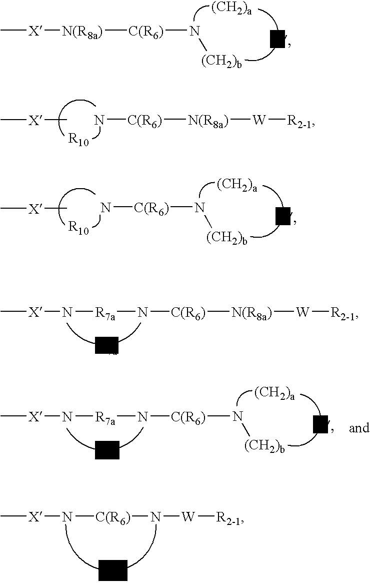
wherein R₇ₐ is C₂₋₃ alkylene, R₁₀ is C₃₋₆ alkylene, and a and b are independently integers from 1 to 4 with the proviso that a+b is ≦5.

In some embodiments, R₂ is selected from the group consisting of —X′—N(R₈ₐ)—C(R₆)—N(R₈ₐ)—W—R₂₋₁, X′—N(R₈ₐ)—C(R₆)—N(OR₈ₐ)—R₂₋₁, 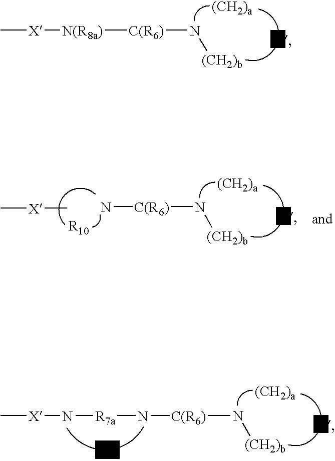
wherein R₇ₐ is C₂₋₃ alkylene, R₁₀ is C₃₋₆ alkylene, and a and b are independently integers from 1 to 4 with the proviso that a+b is ≦5.

In some embodiments, R₂ is selected from the group consisting of —X′—N(R₈ₐ)—C(R₆)—N(R₈ₐ)—W—R₂—, —X′—N(R₈ₐ)—C(R₆)—N(OR₈ₐ)—R₂₋₁, and 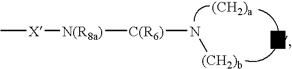
wherein a and b are independently integers from 1 to 4 with the proviso that a+b is ≦5.

In some embodiments, particularly embodiments of Formulas I and II, R₂ is selected from the group consisting of —X′—N(R₈ₐ)—C(R₆)—N(R₈ₐ)—W—R₂₋₁, X′—N(R₈ₐ)—C(R₆)—N(OR₈ₐ)—R₂₋₁, 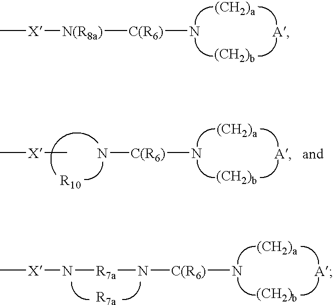
and R₁ is selected from the group consisting of alkyl, arylalkylenyl, heterocyclylalkylenyl, aryloxyalkylenyl, hydroxyalkylenyl, aminoalkylenyl, haloalkylenyl, alkylsulfonylalkylenyl, —X—Y—R₄, and —X—R₅; wherein X is alkylene; Y is —N(R₈)—C(O)—, —N(R₈)—S(O)₂—, —N(R₈)—C(O)—N(R₈)—, —N(R₈)—C(S)—N(R₈)—, —N(R₈)—S(O)₂—N(R₉)—, or 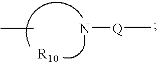
R₄ is alkyl, aryl, or heteroaryl; and R₅ is

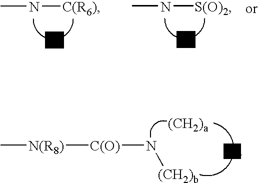

In some embodiments, particularly embodiments of Formulas I, II, III, IV, V, VI, VII, and VIII, R₂ is —X′—N(R₈ₐ)—C(R₆)—N(R₈ₐ)—W—R₂₋₁; R₂₋₁ is selected from the group consisting of C₁₋₄ alkyl, aryl, or substituted aryl wherein the substituent is C₁₋₄ alkyl, C₁₋₄ alkoxy, or halogen; and R₁ is selected from the group consisting of alkyl, arylalkylenyl, heterocyclylalkylenyl, aryloxyalkylenyl, hydroxyalkylenyl, aminoalkylenyl, haloalkylenyl, alkylsulfonylalkylenyl, —X—Y—R₄, and —X—R₅; wherein X is alkylene; Y is —N(R₈)—C(O)—, —N(R₈)—S(O)₂—, —N(R₈)—C(O)—N(R₈)—, —N(R₈)—C(S)—N(R₈)—, —N(R₈)—S(O)₂—N(R₈)—, or 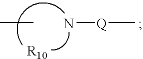
R₄ is alkyl, aryl, or heteroaryl; and R₅ is

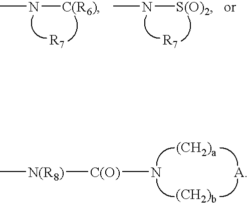

In some embodiments, particularly embodiments of Formulas I, II, III, IV, V, VI, VII, and VIII, R₂ is —X′—N(R₈ₐ)—C(R₆)—N(R₈ₐ)—W—R₂₋₁; X′ is C₁₋₄ alkylene, W is a bond, R₂₋₁ is selected from the group consisting of C₁₋₄ alkyl, aryl, or substituted aryl wherein the substituent is C₁₋₄ alkyl, C₁₋₄ alkoxy, or halogen; and R₁ is selected from the group consisting of alkyl and hydroxyalkylenyl.

In some embodiments, particularly embodiments of Formulas III, IV, V, VI, VII, and VIII, R₂ is selected from the group consisting of —X′—N(R₈ₐ)—C(R₆)—N(R₈ₐ)—W—R₂₋₁, —X′—N(R₈ₐ)—C(R₆)—N(OR₈ₐ)—R₂₋₁, 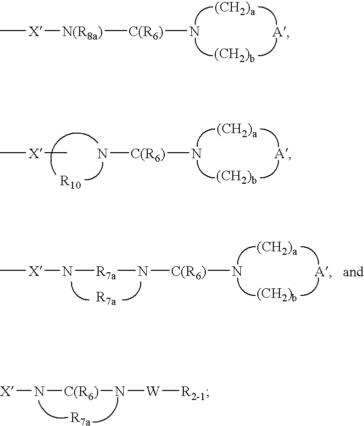
and R₁ is selected from the group consisting of alkyl, arylalkylenyl, heterocyclylalkylenyl, aryloxyalkylenyl, hydroxyalkylenyl, aminoalkylenyl, haloalkylenyl, alkylsulfonylalkylenyl, —X—Y—R₄, and —X—R₅; wherein X is alkylene; Y is —N(R₈)—C(O)—, —N(R₈)—S(O)₂—, —N(R₈)—C(O)—N(R₈)—, —N(R₈)—C(S)—N(R₈)—, —N(R₉)—S(O)₂—N(R₈)—, or 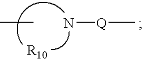
R₄ is alkyl, aryl, or heteroaryl; and R₅ is

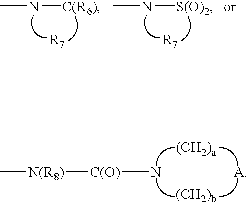

In certain embodiments, Y is selected from the group consisting of —S(O)₀₋₂—, —C(R₆)—, —C(R₆)—O—, —O—C(R₆)—, —O—C(O)—O—, —N(R₈)-Q-, —O—C(R₆)—N(R₈)—, —C(R₆)—N(OR₉)—, and 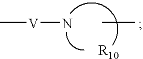
and R₅ is

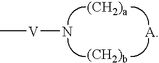

### Preparation of the Compounds

Compounds of the invention may be synthesized by synthetic routes that include processes analogous to those well known in the chemical arts, particularly in light of the description contained herein. The starting materials are generally available from commercial sources such as Aldrich Chemicals (Milwaukee, Wis., USA) or are readily prepared using methods well known to those skilled in the art (e.g. prepared by methods generally described in Louis F. Fieser and Mary Fieser, Reagents for Organic Synthesis, v. 1-19, Wiley, New York, (1967-1999 ed.); Alan R. Katritsky, Otto Meth-Cohn, Charles W. Rees, Comprehensive Organic Functional Group Transformations, v 1-6, Pergamon Press, Oxford, England, (1995); Barry M. Trost and Ian Fleming, Comprehensive Organic Synthesis, v. 1-8, Pergamon Press, Oxford, England, (1991); or Beilsteins Handbuch der organischen Chemie, 4, Aufl. Ed. Springer-Verlag, Berlin, Germany, including supplements (also available via the Beilstein online database)).

For illustrative purposes, the reaction schemes depicted below provide potential routes for synthesizing the compounds of the present invention as well as key intermediates. For more detailed description of the individual reaction steps, see the EXAMPLES section below. Those skilled in the art will appreciate that other synthetic routes may be used to synthesize the compounds of the invention. Although specific starting materials and reagents are depicted in the reaction schemes and discussed below, other starting materials and reagents can be easily substituted to provide a variety of derivatives and/or reaction conditions. In addition, many of the compounds prepared by the methods described below can be further modified in light of this disclosure using conventional methods well known to those skilled in the art.

In the preparation of compounds of the invention it may sometimes be necessary to protect a particular functionality while reacting other functional groups on an intermediate. The need for such protection will vary depending on the nature of the particular functional group and the conditions of the reaction step. Suitable amino protecting groups include acetyl, trifluoroacetyl, tert-butoxycarbonyl (Boc), benzyloxycarbonyl, and 9-fluorenylmethoxycarbonyl (Fmoc). Suitable hydroxy protecting groups include acetyl and silyl groups such as the tert-butyl dimethylsilyl group. For a general description of protecting groups and their use, see T. W. Greene and P. G. M. Wuts, Protective Groups in Organic Synthesis, John Wiley & Sons, New York, USA, 1991.

Conventional methods and techniques of separation and purification can be used to isolate compounds of the invention or pharmaceutically acceptable salts thereof, as well as various intermediates related thereto. Such techniques may include, for example, all types of chromatography (high performance liquid chromatography (HPLC), column chromatography using common absorbents such as silica gel, and thin layer chromatography, recrystallization, and differential (i.e., liquid-liquid) extraction techniques.

Compounds of the invention can be prepared according to Reaction Scheme I where Rₐ, X′, and n are as defined above; Hal is chloro, bromo, or iodo; R₂ₐ is —X′—N(R₈ₐ)C(R₆)—N(R₈ₐ)—W—R₂₋₁, —X′—N(R₈ₐ)—C(R₆)—O—R₂₋₁, or 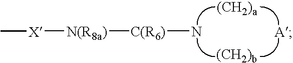
and R₁ₐ is a subset of R₁ as defined above that does not include those substituents that one skilled in the art would recognize as being susceptible to oxidation in step (2). These substituents include —S— and heteroaryl groups.

In step (1) of Reaction Scheme I, a quinoline-3,4-diamine of Formula XX is reacted with a carboxylic acid or carboxylic acid equivalent to provide a 1H-imidazo[4,5-c]quinoline of Formula XXI. The carboxylic acid or carboxylic acid equivalent is selected such that it will provide the desired Hal-X′— substituent in a compound of Formula XXI. Suitable carboxylic acid equivalents include orthoesters of Formula Hal-X′—C(O-alkyl)₃, 1,1-dialkoxyalkyl alkanoates of Formula Hal-X′—C(O-alkyl)₂(O—C(O)-alkyl), and acid halides of Formula Hal-X′—C(O)Cl or Hal-X′—C(O)Br.

The reaction with an acid halide of Formula Hal-X′—C(O)Cl, such as chloroacetyl chloride, is conveniently carried out by combining the acid halide with a quinoline-3,4-diamine of Formula XX in an inert solvent such as dichloromethane in the presence of a base such as triethylamine. The reaction can be carried out at ambient temperature, and the product can be isolated by conventional methods. The reaction may alternatively be carried out in two steps by first adding the acid halide of Formula Hal-X′—C(O)Cl to a solution of the quinoline-3,4-diamine of Formula XX in a suitable solvent such as dichloromethane at a sub-ambient temperature such as 0° C. The amide intermediate can optionally be isolated using conventional techniques and then treated with a base such as triethylamine or aqueous potassium carbonate in a suitable solvent such as dichloromethane, 1,2-dichloroethane, or ethanol or solvent system such as ethanol and water. The cyclization can be carried out at ambient temperature or at an elevated temperature such as the reflux temperature of the solvent.

Many compounds of Formula XX are known and can be readily prepared using known synthetic routes; see for example, U.S. Pat. Nos. 4,689,338 (Gerster), 4,929,624 (Gerster et al.), 5,268,376 (Gerster), 5,389,640 (Gerster et al.), 6,331,539 (Crooks et al.), 6,451,810 (Coleman et al.), 6,541,485 (Crooks et al.), 6,660,747 (Crooks et al.), 6,670,372 (Charles et al.), 6,683,088 (Crooks et al.), 6,656,938 (Crooks et al.), 6,664,264 (Dellaria et al.), and U.S. Patent Publication Application No. US 2004/0147543 (Hays et al.).

In step (2) of Reaction Scheme I a 1H-imidazo[4,5-c]quinoline of Formula XXI is oxidized to a 1H-imidazo[4,5-c]quinoline-5N-oxide of Formula XXII using a conventional oxidizing agent capable of forming N-oxides. The reaction is conveniently carried out at ambient temperature by adding 3-chloroperoxybenzoic acid to a solution of a compound of Formula XXI in a solvent such as chloroform or dichloromethane.

In step (3) of Reaction Scheme I a 1H-imidazo[4,5-c]quinoline-5N-oxide of Formula XXII is aminated to provide an amide-substituted 1H-imidazo[4,5-c]quinolin-4-amine of Formula XXIII. Step (3) can be carried out by the activation of an N-oxide of Formula XXII by conversion to an ester and then reacting the ester with an aminating agent. Suitable activating agents include alkyl- or arylsulfonyl chlorides such as benzenesulfonyl chloride, methanesulfonyl chloride, or p-toluenesulfonyl chloride. Suitable aminating agents include ammonia, in the form of ammonium hydroxide, for example, and ammonium salts such as ammonium carbonate, ammonium bicarbonate, and ammonium phosphate. The reaction is conveniently carried out by adding ammonium hydroxide to a solution of the N-oxide of Formula XXII in a suitable solvent such as dichloromethane or chloroform and then adding p-toluenesulfonyl chloride. The reaction can be carried out at ambient temperature.

Steps (2) and (3) of Reaction Scheme I may be carried out as a one-pot procedure by adding 3-chloroperoxybenzoic acid to a solution of a compound of Formula XXI in a solvent such as dichloromethane or chloroform and then adding ammonium hydroxide and p-toluenesulfonyl chloride without isolating the N-oxide compound of Formula XXII.

In step (4) of Reaction Scheme I a 1H-imidazo[4,5-c]quinoline-4-amine of Formula XXIII is treated with potassium phthalimide to provide a phthalimide-substituted 1H-imidazo[4,5-c]quinolin-4-amine of Formula XIIa. The reaction is conveniently carried out by combining potassium phthalimide and a 1H-imidazo[4,5-c]quinoline-4-amine of Formula XXIII in a suitable solvent such as N,N-dimethylformamide (DMF). The reaction can be carried out at ambient temperature.

In step (5) of Reaction Scheme I a phthalimide-substituted 1H-imidazo[4,5-c]quinolin-4-amine of Formula XIIa is deprotected to an aminoalkyl-1H-imidazo[4,5-c]quinolin-4-amine of Formula XXIV. Removal of the phthalimide protecting group is conveniently carried out by adding hydrazine to a suspension of a phthalimide-protected 1H-imidazo[4,5-c]quinolin-4-amine of Formula XIIa in a suitable solvent such as ethanol. The reaction can be carried out at ambient temperature.

In step (6) of Reaction Scheme I, an aminoalkyl-1H-imidazo[4,5-c]quinolin-4-amine of Formula XXIV is converted to a urea of Formula IIIa, a subgenus of Formulas I and III. For ureas of Formula IIIa, R₂ₐ is —X′—N(R₈ₐ)C(R₆)—N(R₈ₐ)—W—R₂₋₁ or 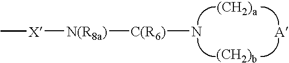
and R₈ₐ, R₆, W, R₂₋₁, a, b, and A′ are as defined above. Compounds of Formula IIIa, where R₂ₐ is —X′—N(R₈ₐ)C(R₆)—N(R₈ₐ)—W—R₂₋₁ and W is a bond, can be prepared by reacting an aminoalkyl-1H-imidazo[4,5-c]quinolin-4-amine of Formula XXIV or pharmaceutically acceptable salt thereof with isocyanates of Formula R₂₋₁N═C═O, isothiocyanates of Formula R₂₋₁N═C═S, or carbamoyl chlorides of Formula R₂₋₁N—(R₈ₐ)—C(R₆)Cl. Many of these isocyanates, isothiocyanates, and carbamoyl chlorides are commercially available; others can be readily prepared using known synthetic methods. The reaction is conveniently carried out by combining the isocyanate of Formula R₂₋₁N═C═O, isothiocyanate of Formula R₂₋₁N═C═S, or carbamoyl chloride of Formula R₄N—(R₈ₐ)—C(R₆)Cl with a solution of the aminoalkyl-1H-imidazo[4,5-c]quinolin-4-amine of Formula XXIV in a suitable solvent such as DMF, chloroform, dichloromethane, N,N-dimethylacetamide (DMA), or pyridine at or below room temperature. Optionally a base such as triethylamine or N,N-diisopropylethylamine can be present. Alternatively, a compound of Formula XXIV can be treated with an isocyanate of Formula R₂₋₁(CO)N═C═O, a thioisocyanate of Formula R₂₋₁(CO)N═C═S, or a sulfonyl isocyanate of Formula R₂₋₁S(O)₂N═C═O using the same method to provide a compound of Formula IIIa, where W is —(CO)— or —S(O)₂—.

Compounds of Formula IIIa where R₂ₐ is 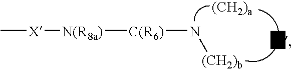
can be prepared according to step (6) by reacting an aminoalkyl-1H-imidazo[4,5-c]quinolin-4-amine of Formula XXIV or pharmaceutically acceptable salt thereof with a carbamoyl chloride of Formula 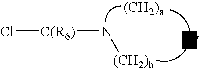
under the conditions described above.

Carbamates of Formula IIIa, wherein R₂ₐ is —X′—N(R₈ₐ)—C(R₆)—O—R₂₋₁, can be prepared in step (6) of Reaction Scheme I by reacting an aminoalkyl-1H-imidazo[4,5-c]quinolin-4-amine of Formula XXIV or pharmaceutically acceptable salt thereof with a chloroformate of Formula Cl—C(O)—O—R₂₋₁ or a carbonic acid anhydride of Formula R₂₋₁—O—C(O)—O—C(O)—O—R₂₋₁. The reaction is conveniently carried out by combining the chloroformate or carbonic acid anhydride with a solution of the aminoalkyl-1H-imidazo[4,5-c]quinolin-4-amine of Formula XXIV in a suitable solvent such as tetrahydrofuran, chloroform, DMF, or DMA in the presence of a base such as triethylamine or N,N-diisopropylethylamine. The reaction may be carried out at a reduced temperature such as 0° C. or at room temperature. Several chloroformates and carbonic acid anhydrides are commercially available; others can be prepared by known synthetic methods.

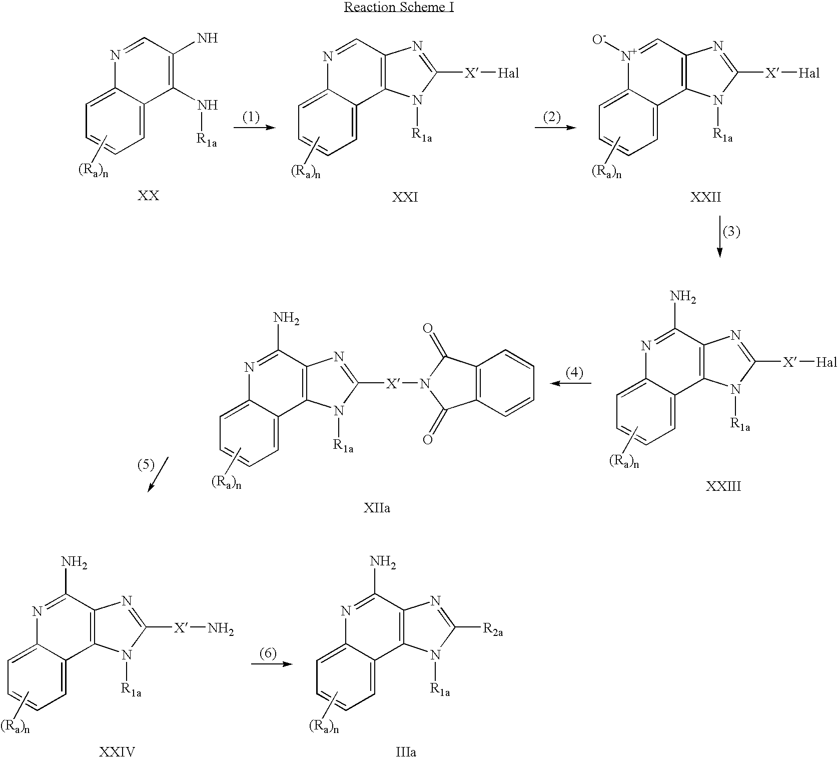

Compounds of the invention can be prepared according to Reaction Scheme II where R₁ₐ, R₇ₐ, Rₐ, X′, Hal, and n are as defined above; R_{2b} is 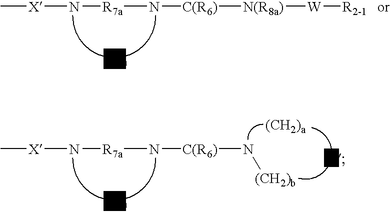
and R₈ₐ, R₆, W, R₂₋₁, a, b, and A′ are as defined above.

In step (1) of Reaction Scheme II, a 1H-imidazo[4,5-c]quinoline-4-amine of Formula XXIII is treated with a cyclic diamine of Formula 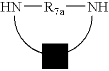
in the presence of a base such as triethylamine or N,N-diisopropylethylamine. Such cyclic diamines, for example piperazine, are commercially available or can be readily synthesized by known methods. The reaction is conveniently carried out in a suitable solvent such as acetonitrile at an elevated temperature such as the reflux temperature of the solvent.

In step (2) of Reaction Scheme II, a 1H-imidazo[4,5-c]quinolin-4-amine of Formula XXV is converted to a urea of Formula IIIb, a subgenus of Formulas I and III. The reaction can be carried out by treating a compound of Formula XXV with an isocyanate, an isothiocyanate, a sulfonyl isocyanate, or a carbamoyl chloride according to one of the methods described in step (6) of Reaction Scheme I.

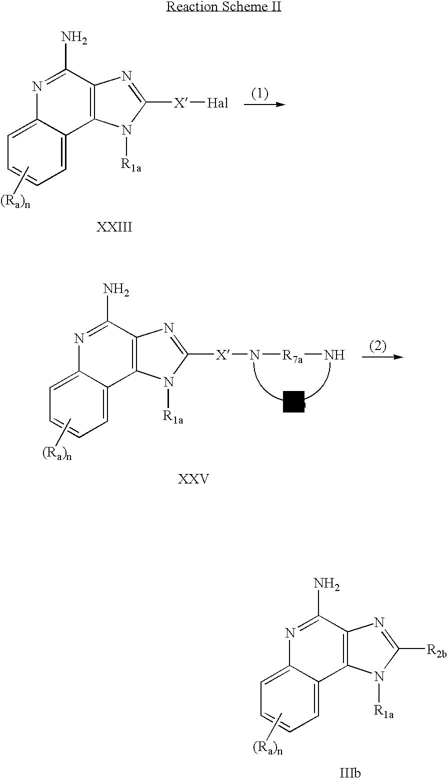

For some embodiments, compounds of the invention can be prepared according to Reaction Scheme III, wherein R₁ₐ, Rₐ, X′, R₈ₐ, R₂₋₁, and n are as defined above, and Ph is phenyl. In step (1) of Reaction Scheme III, an aminoalkyl-1H-imidazo[4,5-c]quinolin-4-amine of Formula XXIV is converted to a carbamate of Formula XXVI, a subgenus of Formulas I and III. The reaction is conveniently carried out by adding phenyl chloroformate to a solution of the compound of Formula XXIV in a suitable solvent such as tetrahydrofuran in the presence of a base such as aqueous sodium bicarbonate.

In step (2) of Reaction Scheme III, the carbamate of Formula XXVI is converted to a urea of Formula IIIc, a subgenus of Formulas I and III. The reaction is conveniently carried out by adding a hydroxylamine of Formula R₂₋₁NHOR₈ₐ or hydroxylamine salt of Formula R₂₋₁NHOR₈ₐ—HCl to a solution of the carbamate of Formula XXVI in a suitable solvent such as dichloromethane. The reaction is run in the presence of a base such as triethylamine. Many hydroxylamine and hydroxylamine salts are commercially available; others can be prepared by known synthetic methods.

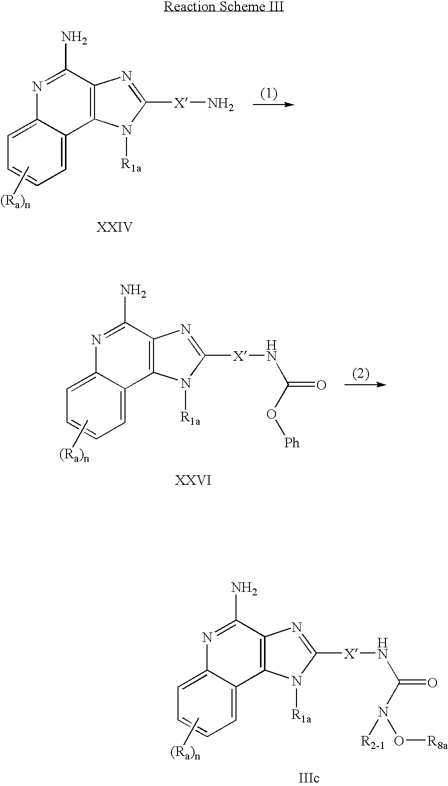

For some embodiments, compounds of the invention are prepared according to Reaction Scheme IV, wherein R₂ₐ, Rₐ, X′, Q, Hal, R₈ₐ, R₄, and n are as defined above, and Boc is a tert-butoxycarbonyl group. In steps (1) through (3) of Reaction Scheme IV, a quinoline-3,4-diamine of Formula XXVII is cyclized to a 1H-imidazoquinoline of Formula XXVIII, which is then oxidized and aminated to a 1H-imidazoquinolin-4-amine of Formula XXX. Steps (1) through (3) of Reaction Scheme IV can be carried out as described for steps (1) through (3) of Reaction Scheme I. Compounds of Formula XXVII are known and can be readily prepared using known synthetic routes; see for example, U.S. Pat. Nos. 6,331,539 (Crooks et al.), 6,451,485 (Crooks et al.), 6,451,810 (Coleman et al.), and 6,677,349 (Griesgraber).

In step (4) of Reaction Scheme IV, a halogen-substituted 1H-imidazo[4,5-c]quinolin-4-amine of Formula XXX is aminated to provide an aminoalkyl-1H-imidazo[4,5-c]quinolin-4-amine of Formula XXXI. The reaction is conveniently carried out by adding a solution of ammonia in a suitable solvent such as methanol to a compound of Formula XXXI. The reaction can be carried out at ambient temperature.

In step (5) of Reaction Scheme IV, an aminoalkyl-1H-imidazo[4,5-c]quinolin-4-amine of Formula XXXI is converted to a urea of Formula IIId, a subgenus of Formulas I and III. The reaction is conveniently carried out as described in step (6) of Reaction Scheme I.

In step (6) of Reaction Scheme IV, the Boc group of the compound of Formula IIId is removed to provide a 1-aminoalkyl-1H-imidazo[4,5-c]quinolin-4-amine of Formula IIIe, which is a subgenus of Formulas I and III. The deprotection is conveniently carried out by adding a solution of hydrogen chloride in a suitable solvent such as dioxane to a solution of the compound of Formula IIId in a suitable solvent or solvent mixture such as methanol and dichloromethane. The reaction can be carried out at ambient temperature.

In step (7) of Reaction Scheme IV, a 1-aminoalkyl-1H-imidazo[4,5-c]quinolin-4-amine of Formula IIIe is converted to a 1H-imidazo[4,5-c]quinolin-4-amine compound of Formula IIIf using conventional methods. For example, an 1-aminoalkyl-1H-imidazo[4,5-c]quinolin-4-amine of Formula IIIe or a salt thereof can react with an acid chloride of Formula R₄C(O)CI to provide a compound of Formula IIIf in which Q is —C(O)—. In addition, a 1H-imidazo[4,5-c]quinolin-4-amine of Formula IIIe can react with sulfonyl chloride of Formula R₄S(O)₂Cl or a sulfonic anhydride of Formula (R₄S(O)₂)₂₋₀ to provide a compound of Formula IIIf in which Q is —S(O)₂—. Numerous acid chlorides of Formula R₄C(O)Cl, sulfonyl chlorides of Formula R₄S(O)₂Cl, and sulfonic anhydrides of Formula (R₄S(O)₂)₂₋₀ are commercially available; others can be readily prepared using known synthetic methods. The reaction is conveniently carried out by adding the acid chloride of Formula R₄C(O)CI, sulfonyl chloride of Formula R₄S(O)₂Cl, or sulfonic anhydride of Formula (R^{S}(O)₂)₂₋₀ to a solution of the 1-aminoalkyl-1H-imidazo[4,5-c]quinolin-4-amine of Formula IIIe in a suitable solvent such as chloroform, dichloromethane, or DMF. Optionally a base such as triethylamine or N,N-diisopropylethylamine can be added. The reaction can be carried out at ambient temperature or a sub-ambient temperature such as 0° C.

Sulfamides of Formula IIIf, where Q is —S(O)₂—N(R₈)—, can be prepared by reacting a compound or salt of Formula IIIe with sulfuryl chloride to generate a sulfamoyl chloride in situ, and then reacting the sulfamoyl chloride with an amine of formula HN(R₈)R₄. Alternatively, sulfamides of Formula IIIf can be prepared by reacting a compound of Formula IIIe with a sulfamoyl chloride of formula R₄(R)N—S(O)₂Cl. Many sulfonyl chlorides of Formula R₄S(O)₂Cl and amines of Formula HN(R₈)R₄, and some sulfamoyl chlorides of Formula R₄(R₈)N—S(O)₂Cl are commercially available; others can be prepared using known synthetic methods.

Compounds of Formula IIIf, wherein Q is —C(O)—N(R₈)—, —C(O)—N(R₈)—(CO)—, —C(S)—N(R₉)—, or —C(O)—N(R₈)—S(O)₂— can be prepared according to one of the methods described step (6) of Reaction Scheme I by reacting a compound of Formula IIIe with an isocyanate or carbamoyl chloride, an isothiocyanate, or a sulfonyl isocyanate.

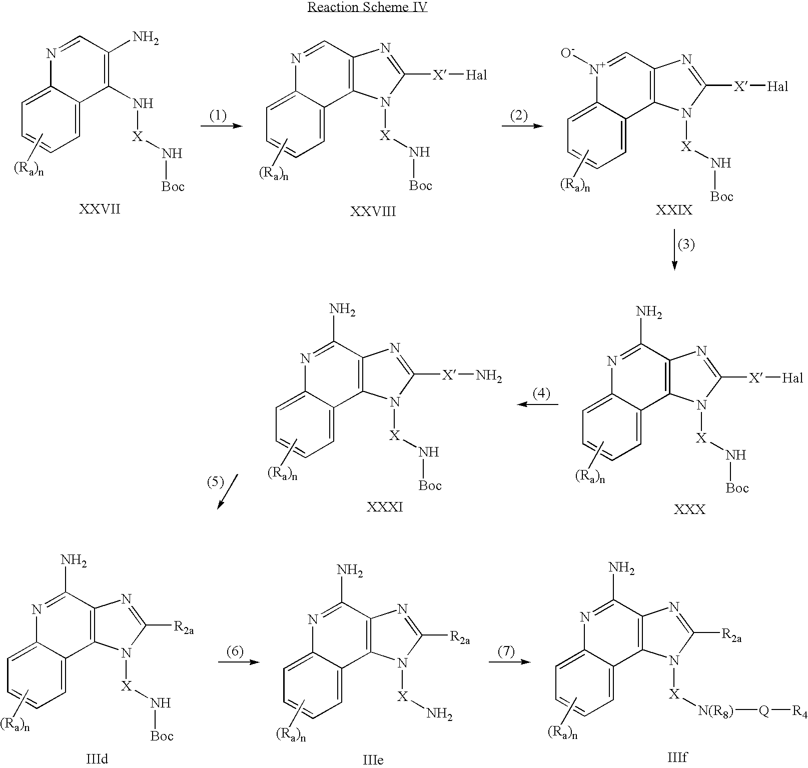

Compounds of the invention can be prepared according to Reaction Scheme V where R₁ₐ, R₁₀, Rₐ, X′, and n are as defined above; R_{2c} is 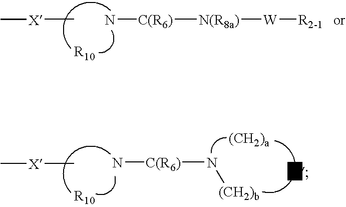
and R₈ₐ, R₆, W, R₂₋₁, a, b, A′, and Boc are as defined above.

In step (1) of Reaction Scheme V, a quinoline-3,4-diamine of Formula XX is reacted with a carboxylic acid of Formula XXXII or XXXIII to provide a 1H-imidazo[4,5-c]quinoline of Formula XXXIV. The reaction can be carried out by heating the quinoline-3,4-diamine of Formula XX with the carboxylic acid of Formula XXXII or XXXIII in polyphosphoric acid or glacial acetic acid. Carboxylic acids of Formula XXXII or XXXIII can be prepared from commercially available starting materials, such as 4-piperidineethanol, using conventional oxidation methods in combination with the methods described in step (6) of Reaction Scheme I.

In step (2) of Reaction Scheme V, the 1H-imidazo[4,5-c]quinoline of Formula XXXIV is first oxidized to a compound of Formula XXXV, which is aminated in step (3) to provide a 1H-imidazo[4,5-c]quinolin-4-amine of Formula IIIg, which is a subgenus of Formulas I and III. Steps (2) and (3) of Reaction Scheme V can be carried out as described in steps (2) and (3) of Reaction Scheme I.

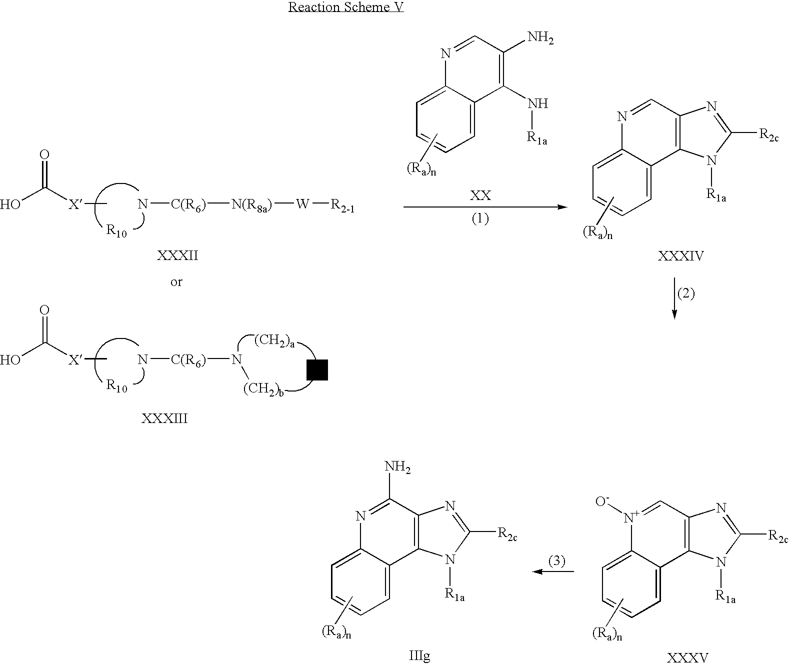

Compounds of the invention can also be prepared according to Reaction Scheme VI, wherein n is as defined above; R_{d} is alkyl, alkoxy, or —N(R₉)₂; and R_{2d} and R_{1d} are subsets of R₁ and R₂ as defined above that do not include those substituents that one skilled in the art would recognize as being susceptible to reduction under the acidic hydrogenation conditions of the reaction. These susceptible groups include, for example, alkenyl, alkynyl, and aryl groups and groups bearing nitro substituents. Compounds of Formula IIIh can be prepared according to any of the methods described in Reaction Schemes I through V.

As shown in Reaction Scheme VI, an 1H-imidazo[4,5-c]quinoline of Formula IIIh can be reduced to a 6,7,8,9-tetrahydro-1H-imidazo[4,5-c]quinolin-4-amine of Formula IVa. The reaction is conveniently carried out under hetereogeneous hydrogenation conditions by adding platinum (IV) oxide to a solution of the compound of Formula IIIh in trifluoroacetic acid and placing the reaction under hydrogen pressure. The reaction can be carried out on a Parr apparatus at ambient temperature.

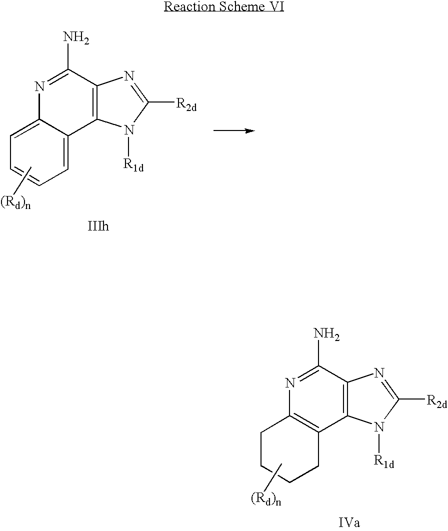

Imidazopyridines of the invention can be prepared according to Reaction Scheme VII, where R₁, R_{A1}, R_{B1}, Ph, X′, Hal, and R₂ₐ are as defined above. In step (1) of Reaction Scheme VII, a 2-phenoxypyridine-3,4-diamine of Formula XXXVII is converted to a 1H-imidazo[4,5-c]pyridine of Formula XXXVIII by reaction with a halogen-substituted carboxylic acid equivalent. The reaction can be carried out as described in step (1) of Reaction Scheme I. When X′ is methylene, the reaction is conveniently carried out by combining a 2-phenoxypyridine-3,4-diamine of Formula XXXVII with ethyl chloroacetimidate hydrochloride in a suitable solvent such as chloroform. The reaction can be carried out at an elevated temperature such as 60° C. Several 2-phenoxypyridine-3,4-diamines of Formula XXXVII are known or can be prepared by published methods. See, for example, U.S. Pat. Nos. 6,545,016 (Dellaria et al.), 6,743,920 (Lindstrom et al.), and 6,797,718 (Dellaria et al.). Ethyl chloroacetimidate hydrochloride is a known compound that can be prepared according to the literature procedure: Stillings, M. R. et al., J. Med. Chem., 29, pp. 2280-2284 (1986).

In step (2) of Reaction Scheme VII, a halogen-substituted 1H-imidazo[4,5-c]pyridine of Formula XXXVIII is aminated to provide an aminoalkyl-1H-imidazo[4,5-c]pyridin-4-amine of Formula XXXIX. The reaction is conveniently carried out by adding a solution of ammonia in a suitable solvent such as methanol to a compound of Formula XXXVIII and heating the reaction at an elevated temperature such as 150° C.

Alternatively, a halogen-substituted 1H-imidazo[4,5-c]pyridine of Formula XXXVIII can be treated according to steps (2a), (2b), and (2c), in which Hal is converted to a phthalimide group and subsequently to an aminoalkyl group in steps (2a) and (2b). Steps (2a) and (2b) can be carried out according to the procedures described in steps (4) and (5) of Reaction Scheme I. The amination shown in step (2c) can be carried out according to the procedure as described in step (2).

In step (3) of Reaction Scheme VII, an aminoalkyl-1H-imidazo[4,5-c]pyridin-4-amine of Formula XXXIX is converted to a urea of Formula IIa, a subgenus of Formulas I and II. The reaction can be carried out according to the methods described in step (6) of Reaction Scheme I.

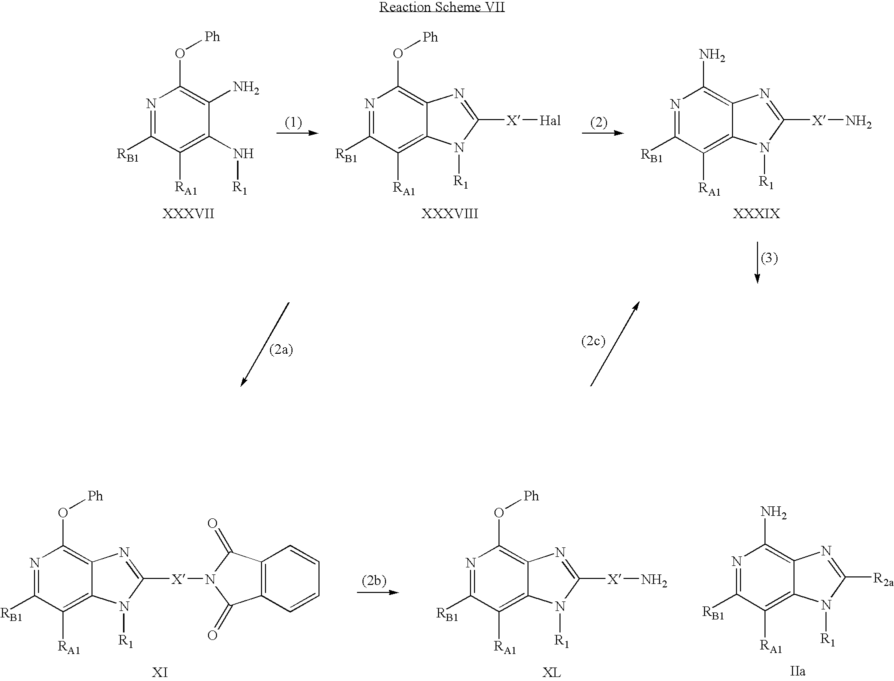

Imidazonaphthyridines of the invention can be prepared according to Reaction Scheme VIII, wherein R_{b}, X′, R₁ₐ, R₂ₐ Hal, and m are as defined above. Reaction Scheme VIII begins with a [1,5]naphthyridine-3,4-diamine of Formula XLI. Compounds of Formula XLI and their preparation are known; see, for example, U.S. Pat. Nos. 6,194,425 (Gerster) and 6,518,280 (Gerster). Steps (1) through (6) of Reaction Scheme VIII can be carried out as described for the corresponding steps (1) through (6) of Reaction Scheme I to provide a urea-substituted 1H-imidazo[4,5-c][1,5]naphthyridin-4-amine of Formula Va.

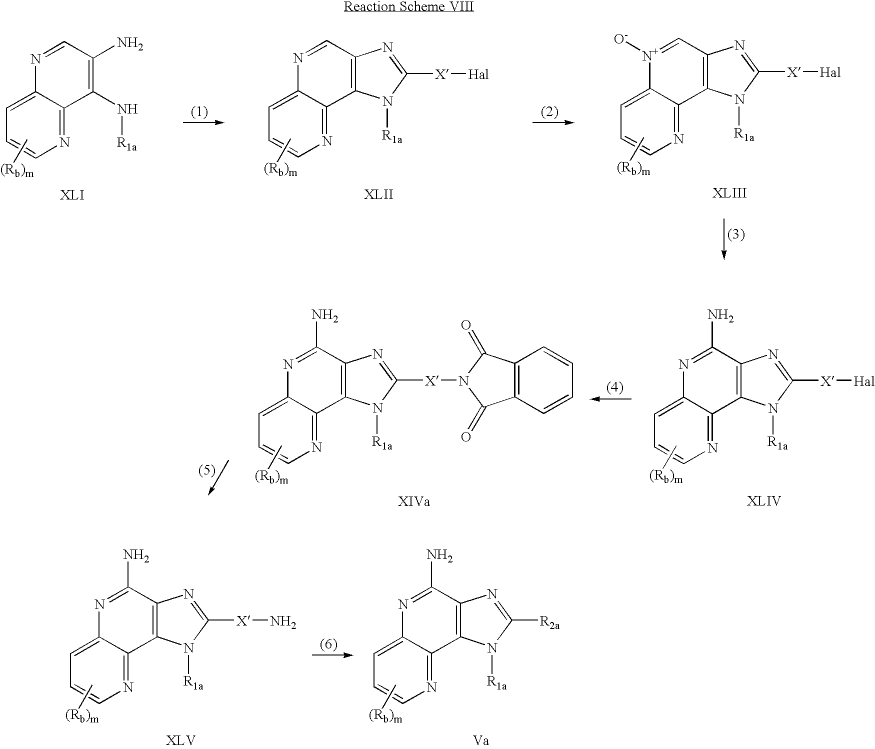

For some embodiments, naphthyridines of the invention are prepared from tetrazolo compounds of Formulas XLVI and XLIX according to Reaction Scheme IX and X, wherein R₁, R₂ₐ, Hal, R_{b}, m, and X′ are as defined above. Compounds of Formula XLVI and XLIX and synthetic routes to these compounds are known; see, for example, U.S. Pat. Nos. 6,194,425 (Gerster) and 6,518,280 (Gerster).

In step (1) of Reaction Scheme IX and X, a tetrazolonaphthyridine of Formula XLVI or XLIX is reacted with a halogen-substituted carboxylic acid or equivalent thereof to form a compound of Formula XLVII or L. The reaction can be carried out as described in step (1) of Reaction Scheme I. A halogen-substituted 1H-imidazo[4,5-c][1,5]naphthyridine of Formula XLVII or L is converted to a compound of Formula XLVIII or LI according to the methods of steps (4), (5), and (6) of Reaction Scheme I. The tetrazolo group of a compound of Formula XLVIII or LI can then be removed to provide a 1H-imidazo[4,5-c]naphthyridin-4-amine of Formula VIIIa or VIIa. The removal of the tetrazolo group can be carried out using methods described in U.S. Pat. Nos. 6,194,425 (Gerster) and 6,518,280 (Gerster).

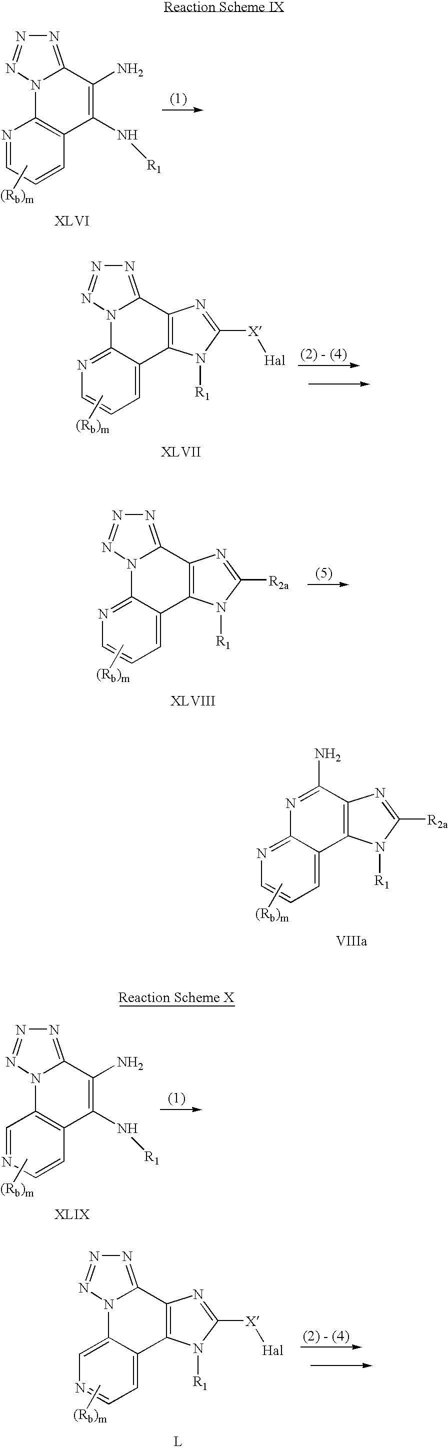

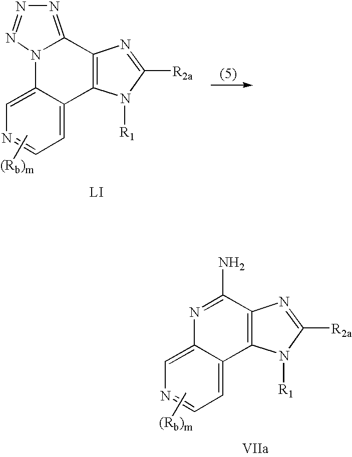

Tetrahydroquinolines of the invention can be prepared according to Reaction Scheme XI, wherein R_{1d}, R_{d}, and n are as defined above, P is a hydroxy protecting group, X′ₐ is C₁₋₄ alkylene, and R₂ₐ-1 is a subset of R₂ₐ as defined above in which X′ is C₁₋₄ alkylene.

In step (1) of Reaction Scheme XI, a compound of Formula XXa or a salt thereof is reacted with a carboxylic acid or an equivalent thereof to provide a compound of Formula LII. Compounds of Formula XXa are a subset of compounds of Formula XX, which are shown in Reaction Scheme I. Suitable carboxylic acid equivalents that can be used to provide a compound of formula LII include acid anhydrides of formula O[C(O)—X′ₐ—CH₂—O—P]₂ and acid chlorides of formula Cl—C(O)—X′ₐ—CH₂—O—P. The reaction is conveniently carried out by under the conditions described in step (1) of Reaction Scheme I for the reaction with acid chlorides of formula Hal-X′—C(O)Cl. Some compounds of formula Cl—C(O)—X′ₐ—O—P, such as acetoxyacetyl chloride, methoxyacetyl chloride, and 2-methoxypropionyl chloride, are commercially available. Others can be prepared by known synthetic methods.

Alternatively, step (1) can be carried out in two steps by first heating a quinoline-3,4-diamine of Formula XXa with a carboxylic acid of formula HO—X′ₐ—CO₂H, with a trialkyl orthoester of formula HO—X′ₐ—C(O—C₁₋₄ alkyl)₃, or with a combination thereof to provide a 2-hydroxyalkyl-1H-imidazo[4,5-c]quinoline. The reaction is run with sufficient heating to drive off any alcohol or water formed as a byproduct of the reaction and is typically run at about 130° C. The resultant hydroxy-substituted compound is protected with a removable protecting group such as an alkanoyloxy group (e.g., acetoxy) or aroyloxy group (e.g., benzoyloxy) to provide a 1H-imidazo[4,5-c]quinoline of Formula LII. Suitable protecting groups and reactions for their placement and removal are well known to those skilled in the art. See, for example, U.S. Pat. No. 4,689,338 (Gerster), Examples 115 and 120 and U.S. Pat. No. 5,389,640 (Gerster et al.), Examples 2 and 3.

In steps (2) and (3) of Reaction Scheme XI, a protected hydroxyalkyl-1H-imidazo[4,5-c]quinoline of Formula LII is first oxidized to an N-oxide of Formula LIII, which is then aminated to a hydroxyalkyl-1H-imidazo[4,5-c]quinolin-4-amine of Formula LIV. Steps (2) and (3) of Reaction Scheme XI can be carried out as described for steps (2) and (3) of Reaction Scheme I. Under the amination reaction conditions, some protecting groups are removed; for example, an ester group such as an acetoxy group would be hydrolyzed under these conditions. Other hydroxy protecting groups may need to be removed in a subsequent step prior to step (4) to provide a compound of Formula LIV. For example, a methyl ether, wherein P is methyl, can be dealkylated by treatment with boron tribromide in a suitable solvent such as dichloromethane at a sub-ambient temperature such as 0° C.

In step (4) of Reaction Scheme XI, a hydroxyalkyl-1H-imidazo[4,5-c]quinolin-4-amine of Formula LIV is reduced according to the method described in Reaction Scheme VI to provide a hydroxyalkyl-6,7,8,9-tetrahydro-1H-imidazo[4,5-c]quinolin-4-amine of Formula LV.

In step (5) of Reaction Scheme XI, a hydroxyalkyl-substituted compound of Formula LV is halogenated using conventional methods to provide a haloalkyl-6,7,8,9-tetrahydro-1H-imidazo[4,5-c]quinolin-4-amine of Formula LVI. For example, a hydroxyalkyl-substituted compound of Formula LV can be combined with thionyl chloride in a suitable solvent such as dichloromethane or 1,2-dichloroethane at room temperature.

In step (6) of Reaction Scheme XI, a haloalkyl-substituted compound of Formula LVI is treated with potassium phthalimide under the conditions described in step (4) of Reaction Scheme I to provide a phthalimide-substituted 6,7,8,9-tetrahydro-1H-imidazo[4,5-c]quinolin-4-amine of Formula XIIIa.

In steps (7) and (8) of Reaction Scheme XI a phthalimide-substituted compound of Formula XIIIa is deprotected to an aminoalkyl-substituted compound of Formula LVII, which is then converted to a urea of Formula IVb. Steps (7) and (8) of Reaction Scheme XI can be carried out according to the methods described in steps (5) and (6) of Reaction Scheme I.

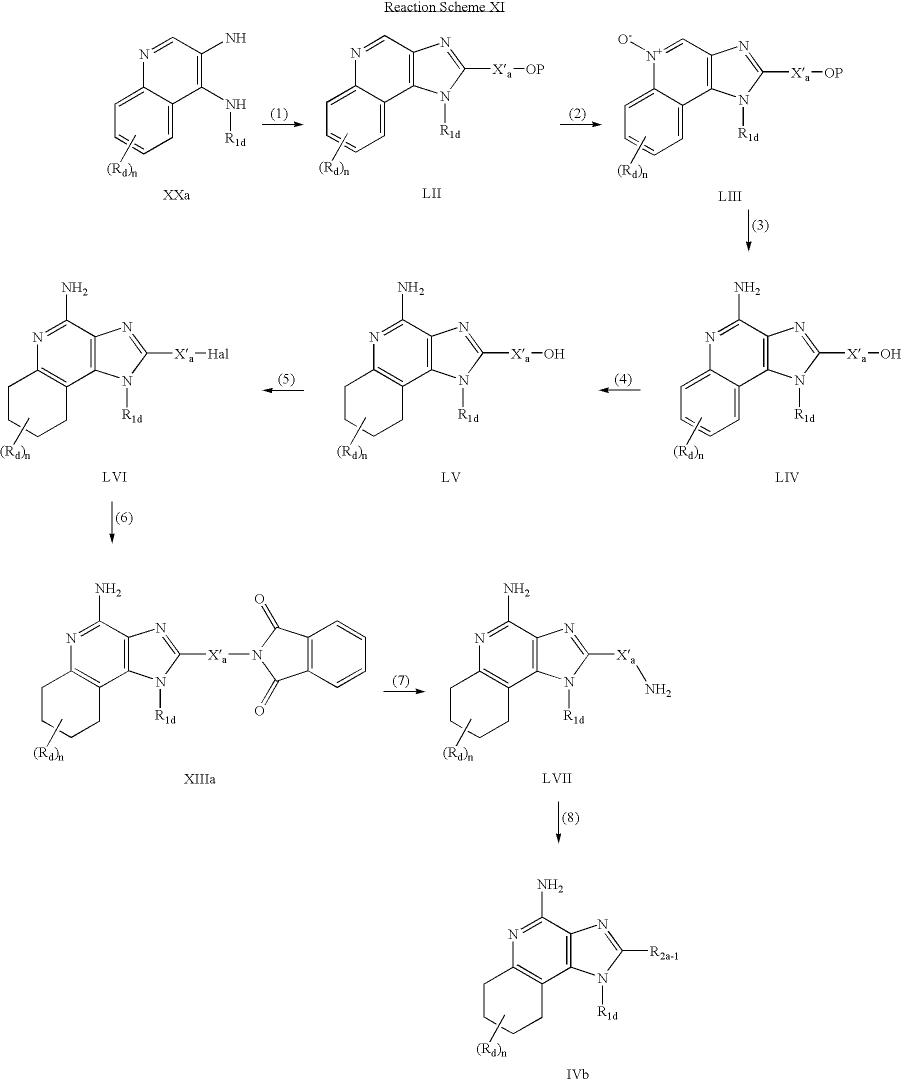

Some compounds of XX or XLI in which R₁ₐ is a 1-hydroxycycloalkylmethyl group can be prepared in two steps by (i) reacting 4-chloro-3-nitroquinoline or 4-chloro-3-nitro[1,5]naphthyridine with an amine of formula H₂N—R₁ₐ or a salt thereof and (ii) reducing the nitro group using conventional methods. Methods that can be used to carry out step (i) and step (ii) are described in the U.S. patents referenced in step (1) of Reaction Scheme I.

Some amines of the Formula H₂N—R₁ₐ in which R₁ₐ is a 1-hydroxycycloalkylmethyl group, or salts thereof, are commercially available. Others can be prepared by combining a cyclic ketone with excess nitromethane in a suitable solvent such as ethanol or methanol in the presence of a catalytic amount of base such as sodium ethoxide or sodium hydroxide and reducing the resultant nitromethyl-substituted compound using conventional heterogeneous hydrogenation conditions. The hydrogenation is typically carried out in the presence of a catalyst such as palladium hydroxide on carbon, palladium on carbon, or Raney nickel in a suitable solvent such as ethanol. Both the reaction with nitromethane and the reduction can be carried out at ambient temperature. A wide variety of cyclic ketones, such as cyclopentanone and cyclobutanone, can be obtained from commercial sources; others can be synthesized using known synthetic methods.

Compounds of the invention can also be prepared using variations of the synthetic routes shown in Reaction Schemes I through XI. For example, naphthyridines XLIV, XLV, and XLI can be used as starting materials for the routes shown in Reaction Schemes II, III, and V, respectively, to prepare compounds of Formula V. Certain naphthyridines of Formula XLI can be used as starting materials for the route shown in Reaction Scheme XI to prepare tetrahydronaphthyridines, and certain naphthyridines of Formula Va can be treated according to Reaction Scheme VI to prepare tetrahydronaphthyridines. Compounds of the invention can also be prepared using the synthetic routes described in the EXAMPLES below.

Prodrugs can be prepared in a variety of ways. For example, a compound wherein

R₁ is —X—OH (e.g. hydroxyalkyl) can be converted into a prodrug wherein R₁ is, for example, —X—O—C(R₆)—R₄, —X—O—C(R₆)—O—R₄, or —X—O—C(R₆)—N(R₈)—R₄, wherein X, R₄, R₆, and R₈ are as defined above, using methods known to one skilled in the art. In addition, a compound wherein R_{b} is hydroxy may also be converted to an ester, an ether, a carbonate, or a carbamate. For any of these compounds containing an alcohol functional group, a prodrug can be formed by the replacement of the hydrogen atom of the alcohol group with a group such as C₁₋₆ alkanoyloxymethyl, 1-(C₁₋₆ alkanoyloxy)ethyl, 1-methyl-1-(C₁₋₆ alkanoyloxy)ethyl, C₁₋₆ alkoxycarbonyloxymethyl, N—(C₁₋₆ alkoxycarbonyl)aminomethyl, succinoyl, C₁₋₆ alkanoyl, α-aminoC₁₋₄ alkanoyl, arylacyl, —P(O)(OH)₂, —P(O)(O—C₁₋₆ alkyl)₂, C₁₋₆ alkoxycarbonyl, C₁₋₆ alkylcarbamoyl, and α-aminoacyl or α-aminoacyl-α-aminoacyl, where each α-aminoacyl group is independently selected from the naturally occurring L-amino acids. For compounds containing an alcohol functional group, particularly useful prodrugs are esters made from carboxylic acids containing one to six carbon atoms, unsubstituted or substituted benzoic acid esters, or esters made from naturally occurring L-amino acids.

Prodrugs can also be made from a compound containing an amino group by conversion of the amino group to a functional group such as an amide, carbamate, urea, amidine, or another hydroylizable group using conventional methods. A prodrug of this type can be made by the replacement of a hydrogen atom in an amino group, particularly the amino group at the 4-position, with a group such as —C(O)—R′, α-aminoacyl, α-aminoacyl-α-aminoacyl, —C(O)—O—R′, —C(O)—N(R″)—R′, —C(═NY′)—R′, —CH(OH)—C(O)—OY′, —CH(OC₁₋₄ alkyl)Y₀, —CH₂Y₁, or —CH(CH₃)Y₁; wherein R′ and R″ are each independently C₁₋₁₀ alkyl, C₃₋₇ cycloalkyl, or benzyl, each of which may be unsubstituted or substituted by one or more substitutents selected from the group consisting of halogen, hydroxy, nitro, cyano, carboxy, C₁₋₆ alkyl, C₁₋₄ alkoxy, aryl, heteroaryl, arylC₁₋₄ alkylenyl, heteroarylC₁₋₄ alkylenyl, haloC₁₋₄ alkyl, haloC₁₋₄ alkoxy, —O—C(O)—CH₃, —C(O)—O—CH₃, —C(O)—NH₂, —O—CH₂—C(O)—NH₂, —NH₂, and —S(O)₂—NH₂; each α-aminoacyl group is independently selected from the naturally occurring L-amino acids; Y′ is hydrogen, C₁₋₆ alkyl, or benzyl; Y₀ is C₁₋₆ alkyl, carboxyC₁₋₆ alkyl, aminoC₁₋₄ alkyl, mono-N—C₁₋₆ alkylaminoC₁₋₄ alkyl, or di-N,N—C₁₋₆ alkylaminoC₁₋₄ alkyl; and Y₁ is mono-N—C₁₋₆ alkylamino, di-N,N—C₁₋₆ alkylamino, morpholin-4-yl, piperidin-1-yl, pyrrolidin-1-yl, or 4-C₁₋₄ alkylpiperazin-1-yl.

Pharmaceutical Compositions and Biological Activity

Pharmaceutical compositions of the invention contain a therapeutically effective amount of a compound or salt of the invention as described above in combination with a pharmaceutically acceptable carrier.

The terms “a therapeutically effective amount” and “effective amount” mean an amount of the compound or salt sufficient to induce a therapeutic or prophylactic effect, such as cytokine induction, immunomodulation, antitumor activity, and/or antiviral activity. Although the exact amount of active compound or salt used in a pharmaceutical composition of the invention will vary according to factors known to those of skill in the art, such as the physical and chemical nature of the compound or salt, the nature of the carrier, and the intended dosing regimen, it is anticipated that the compositions of the invention will contain sufficient active ingredient to provide a dose of about 100 nanograms per kilogram (ng/kg) to about 50 milligrams per kilogram (mg/kg), preferably about 10 micrograms per kilogram (μg/kg) to about 5 mg/kg, of the compound or salt to the subject. A variety of dosage forms may be used, such as tablets, lozenges, capsules, parenteral formulations, syrups, creams, ointments, aerosol formulations, transdermal patches, transmucosal patches and the like.

The compounds or salts of the invention can be administered as the single therapeutic agent in the treatment regimen, or the compounds or salts of the invention may be administered in combination with one another or with other active agents, including additional immune response modifiers, antivirals, antibiotics, antibodies, proteins, peptides, oligonucleotides, etc.

Compounds or salts of the invention have been shown to induce, and certain compounds or salts of the invention may inhibit, the production of certain cytokines in experiments performed according to the tests set forth below. These results indicate that the compounds or salts are useful as immune response modifiers that can modulate the immune response in a number of different ways, rendering them useful in the treatment of a variety of disorders.

Cytokines whose production may be induced by the administration of compounds or salts of the invention generally include interferon-α (IFN-α) and/or tumor necrosis factor-α (TNF-α) as well as certain interleukins (IL). Cytokines whose biosynthesis may be induced by compounds or salts of the invention include IFN-α, TNF-α, IL-1, IL-6, IL-10 and IL-12, and a variety of other cytokines. Among other effects, these and other cytokines can inhibit virus production and tumor cell growth, making the compounds or salts useful in the treatment of viral diseases and neoplastic diseases. Accordingly, the invention provides a method of inducing cytokine biosynthesis in an animal comprising administering an effective amount of a compound or salt or composition of the invention to the animal. The animal to which the compound or salt or composition is administered for induction of cytokine biosynthesis may have a disease as described infra, for example a viral disease or a neoplastic disease, and administration of the compound or salt may provide therapeutic treatment. Alternatively, the compound or salt may be administered to the animal prior to the animal acquiring the disease so that administration of the compound or salt may provide a prophylactic treatment.

In addition to the ability to induce the production of cytokines, compounds or salts of the invention can affect other aspects of the innate immune response. For example, natural killer cell activity may be stimulated, an effect that may be due to cytokine induction. The compounds or salts may also activate macrophages, which in turn stimulate secretion of nitric oxide and the production of additional cytokines. Further, the compounds or salts may cause proliferation and differentiation of B-lymphocytes.

Compounds or salts of the invention can also have an effect on the acquired immune response. For example, the production of the T helper type 1 (T_{H}1) cytokine IFN-γ may be induced indirectly and the production of the T helper type 2 (T_{H}2) cytokines IL-4, IL-5 and IL-13 may be inhibited upon administration of the compounds or salts.

Other cytokines whose production may be inhibited by the administration of compounds or salts of the invention include tumor necrosis factor-α (TNF-α). Among other effects, inhibition of TNF-α production can provide prophylaxis or therapeutic treatment of TNF-α mediated diseases in animals, making the compounds or salt useful in the treatment of, for example, autoimmune diseases. Accordingly, the invention provides a method of inhibiting TNF-α biosynthesis in an animal comprising administering an effective amount of a compound or salt or composition of the invention to the animal. The animal to which the compound or salt or composition is administered for inhibition of TNF-α biosynthesis may have a disease as described infra, for example an autoimmune disease, and administration of the compound or salt may provide therapeutic treatment. Alternatively, the compound or salt may be administered to the animal prior to the animal acquiring the disease so that administration of the compound or salt may provide a prophylactic treatment.

Whether for prophylaxis or therapeutic treatment of a disease, and whether for effecting innate or acquired immunity, the compound or salt or composition may be administered alone or in combination with one or more active components as in, for example, a vaccine adjuvant. When administered with other components, the compound or salt and other component or components may be administered separately; together but independently such as in a solution; or together and associated with one another such as (a) covalently linked or (b) non-covalently associated, e.g., in a colloidal suspension.

Conditions for which compounds or salts identified herein may be used as treatments include, but are not limited to:

(a) viral diseases such as, for example, diseases resulting from infection by an adenovirus, a herpesvirus (e.g., HSV-I, HSV-II, CMV, or VZV), a poxvirus (e.g., an orthopoxvirus such as variola or vaccinia, or molluscum contagiosum), a picornavirus (e.g., rhinovirus or enterovirus), an orthomyxovirus (e.g., influenzavirus), a paramyxovirus (e.g., parainfluenzavirus, mumps virus, measles virus, and respiratory syncytial virus (RSV)), a coronavirus (e.g., SARS), a papovavirus (e.g., papillomaviruses, such as those that cause genital warts, common warts, or plantar warts), a hepadnavirus (e.g., hepatitis B virus), a flavivirus (e.g., hepatitis C virus or Dengue virus), or a retrovirus (e.g., a lentivirus such as HIV);

(b) bacterial diseases such as, for example, diseases resulting from infection by bacteria of, for example, the genus Escherichia, Enterobacter, Salmonella, Staphylococcus, Shigella, Listeria, Aerobacter, Helicobacter, Klebsiella, Proteus, Pseudomonas, Streptococcus, Chlamydia, Mycoplasma, Pneumococcus, Neisseria, Clostridium, Bacillus, Corynebacterium, Mycobacterium, Campylobacter, Vibrio, Serratia, Providencia, Chromobacterium, Brucella, Yersinia, Haemophilus, or Bordetella;

(c) other infectious diseases, such chlamydia, fungal diseases including but not limited to candidiasis, aspergillosis, histoplasmosis, cryptococcal meningitis, or parasitic diseases including but not limited to malaria, pneumocystis carnii pneumonia, leishmaniasis, cryptosporidiosis, toxoplasmosis, and trypanosome infection;

(d) neoplastic diseases, such as intraepithelial neoplasias, cervical dysplasia, actinic keratosis, basal cell carcinoma, squamous cell carcinoma, renal cell carcinoma, Kaposi's sarcoma, melanoma, leukemias including but not limited to myelogeous leukemia, chronic lymphocytic leukemia, multiple myeloma, non-Hodgkin's lymphoma, cutaneous T-cell lymphoma, B-cell lymphoma, and hairy cell leukemia, and other cancers;

(e) T_{H}2-mediated, atopic diseases, such as atopic dermatitis or eczema, eosinophilia, asthma, allergy, allergic rhinitis, and Ommen's syndrome;

(f) certain autoimmune diseases such as systemic lupus erythematosus, essential thrombocythaemia, multiple sclerosis, discoid lupus, alopecia greata; and

(g) diseases associated with wound repair such as, for example, inhibition of keloid formation and other types of scarring (e.g., enhancing wound healing, including chronic wounds).

Additionally, a compound or salt of the present invention may be useful as a vaccine adjuvant for use in conjunction with any material that raises either humoral and/or cell mediated immune response, such as, for example, live viral, bacterial, or parasitic immunogens; inactivated viral, tumor-derived, protozoal, organism-derived, fungal, or bacterial immunogens, toxoids; toxins; self-antigens; polysaccharides; proteins; glycoproteins; peptides; cellular vaccines; DNA vaccines; autologous vaccines; recombinant proteins; and the like, for use in connection with, for example, BCG, cholera, plague, typhoid, hepatitis A, hepatitis B, hepatitis C, influenza A, influenza B, parainfluenza, polio, rabies, measles, mumps, rubella, yellow fever, tetanus, diphtheria, hemophilus influenza b, tuberculosis, meningococcal and pneumococcal vaccines, adenovirus, HIV, chicken pox, cytomegalovirus, dengue, feline leukemia, fowl plague, HSV-1 and HSV-2, hog cholera, Japanese encephalitis, respiratory syncytial virus, rotavirus, papilloma virus, yellow fever, and Alzheimer's Disease.

Compounds or salts of the present invention may be particularly helpful in individuals having compromised immune function. For example, compounds or salts may be used for treating the opportunistic infections and tumors that occur after suppression of cell mediated immunity in, for example, transplant patients, cancer patients and HIV patients.

Thus, one or more of the above diseases or types of diseases, for example, a viral disease or a neoplastic disease may be treated in an animal in need thereof (having the disease) by administering a therapeutically effective amount of a compound or salt of the invention to the animal.

An amount of a compound or salt effective to induce or inhibit cytokine biosynthesis is an amount sufficient to cause one or more cell types, such as monocytes, macrophages, dendritic cells and B-cells to produce an amount of one or more cytokines such as, for example, IFN-α, TNF-α, IL-1, IL-6, IL-10 and IL-12 that is increased (induced) or decreased (inhibited) over a background level of such cytokines. The precise amount will vary according to factors known in the art but is expected to be a dose of about 100 ng/kg to about 50 mg/kg, preferably about 10 μg/kg to about 5 mg/kg. The invention also provides a method of treating a viral infection in an animal and a method of treating a neoplastic disease in an animal comprising administering an effective amount of a compound or salt or composition of the invention to the animal. An amount effective to treat or inhibit a viral infection is an amount that will cause a reduction in one or more of the manifestations of viral infection, such as viral lesions, viral load, rate of virus production, and mortality as compared to untreated control animals. The precise amount that is effective for such treatment will vary according to factors known in the art but is expected to be a dose of about 100 ng/kg to about 50 mg/kg, preferably about 10 μg/kg to about 5 mg/kg. An amount of a compound or salt effective to treat a neoplastic condition is an amount that will cause a reduction in tumor size or in the number of tumor foci. Again, the precise amount will vary according to factors known in the art but is expected to be a dose of about 100 ng/kg to about 50 mg/kg, preferably about 10 μg/kg to about 5 mg/kg.

In addition to the formulations and uses described specifically herein, other formulations, uses, and administration devices suitable for compounds of the present invention are described in, for example, International Publication Nos. WO 03/077944 and WO 02/036592, U.S. Pat. No. 6,245,776, and U.S. Publication Nos. 2003/0139364, 2003/185835, 2004/0258698, 2004/0265351, 2004/076633, and 2005/0009858.

### EXAMPLES

Objects and advantages of this invention are further illustrated by the following examples, but the particular materials and amounts thereof recited in these examples, as well as other conditions and details, should not be construed to unduly limit this invention.

In the examples below normal phase preparative high performance flash chromatography (prep HPLC) was carried out using a COMBIFLASH system (an automated high-performance flash purification product available from Teledyne Isco, Inc., Lincoln, Nebr., USA), a HORIZON HPFC system (an automated high-performance flash purification product available from Biotage, Inc, Charlottesville, Va., USA) or a combination thereof. For some of these purifications, either a FLASH 40+M silica cartridge or a FLASH 65I silica cartridge (both available from Biotage, Inc, Charlottesville, Va., USA) was used. The eluent used for each purification is given in the example. In some chromatographic separations, the solvent mixture 80/18/2 v/v/v chloroform/methanol/concentrated ammonium hydroxide (CMA) was used as the polar component of the eluent. In these separations, CMA was mixed with chloroform in the indicated ratio.

### Example 1

### N-{[4-Amino-1-(2-methylpropyl)-1H-imidazo[4,5-c]quinolin-2-yl]methyl}-N′-methylurea

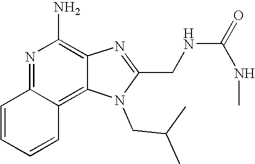

### Part A

N⁴-(2-Methylpropyl)quinoline-3,4-diamine (41 g, 0.190 mol, U.S. Pat. No. 5,389,640 Example 1), dichloromethane (550 mL), triethylamine (40 mL, 0.286 mol), and chloroacetyl chloride (16.7 mL, 0.210 mol) were combined and then stirred at ambient temperature over the weekend. The reaction mixture was diluted with 1,2-dichloroethane (75 mL) and then washed with saturated aqueous sodium bicarbonate (3×400 mL). The organic layer was dried over magnesium sulfate, filtered through a layer of CELITE filter agent, and then concentrated under reduced pressure to provide 52.81 g of 2-(chloromethyl)-1-(2-methylpropyl)-1H-imidazo[4,5-c]quinoline as a brown solid.

### Part B

3-Chloroperoxybenzoic acid (32.7 g of 77% pure material, 146 mmol) was added over a period of five minutes to a solution of 2-(chloromethyl)-1-(2-methylpropyl)-1H-imidazo[4,5-c]quinoline (20.0 g, 73.1 mmol) in chloroform (500 mL); the reaction mixture was stirred at ambient temperature for one hour. Ammonium hydroxide (200 mL) was added, and then p-toluenesulfonyl chloride (16.7 g, 87.7 mmol) was added in portions over a period of 10 minutes. The reaction mixture was stirred at ambient temperature for one hour, and then water (200 mL) was added. The aqueous layer was separated and extracted with dichloromethane (2×200 mL). The combined organic fractions were dried over magnesium sulfate, filtered, and concentrated under reduced pressure to provide 32 g of crude product as a tan solid. The crude product was dissolved in dichloromethane (50 mL), and the resulting solution was divided into two portions. Each portion was purified by prep HPLC on a HORIZON HPFC system using a FLASH 65I silica cartridge (eluting with ethyl acetate:methanol in a gradient from 98:2 to 85:15) to provide 11.24 g of 2-(chloromethyl)-1-(2-methylpropyl)-1H-imidazo[4,5-c]quinolin-4-amine as a tan solid.

### Part C

Potassium phthalimide (6.3 g, 34 mmol) was added to a solution of 2-(chloromethyl)-1-(2-methylpropyl)-1H-imidazo[4,5-c]quinolin-4-amine (8.2 g, 28 mmol) in N,N-dimethylformamide (DMF, 30 mL); a precipitate formed. The reaction mixture was stirred at ambient temperature overnight, and then water (300 mL) was added. The resulting mixture was stirred for 15 minutes, and the precipitate was isolated by filtration, washed with water, and dried overnight in a vacuum oven at 65° C. to provide 9.71 g of 2-{[4-amino-1-(2-methylpropyl)-1H-imidazo[4,5-c]quinolin-2-yl]methyl}-1H-isoindole-1,3(2H)-dione.

### Part D

Hydrazine (1.14 mL, 36.4 mmol) was added to a stirred suspension of 2-{[4-amino-1-(2-methylpropyl)-1H-imidazo[4,5-c]quinolin-2-yl]methyl}-1H-isoindole-1,3(2H)-dione (9.7 g, 24 mmol) in ethanol (200 mL). After 2.5 hours at ambient temperature, an analysis by liquid chromatography/mass spectrometry (LC/MS) indicated the presence of starting material. Additional hydrazine (2 mL) was added, and the reaction was stirred at ambient temperature overnight. The reaction mixture was filtered to remove a precipitate, and the filter cake was washed with dichloromethane. The filtrate was concentrated under reduced pressure, dissolved in methanol:dichloromethane, and purified by prep HPLC on a HORIZON HPFC system using a FLASH 40+M cartridge (eluting with chloroform:2 M ammonia in methanol in a gradient from 95:5 to 85:15) to provide 5.05 g of 2-(aminomethyl)-1-(2-methylpropyl)-1H-imidazo[4,5-c]quinolin-4-amine as a yellow solid.

### Part E

Methyl isocyanate (0.252 mL, 4.08 mmol) was added to a stirred suspension of 2-(aminomethyl)-1-(2-methylpropyl)-1H-imidazo[4,5-c]quinolin-4-amine (1.0 g, 3.7 mmol) in DMF (10 mL), and the resulting solution was stirred at ambient temperature for one hour. Dichloromethane (30 mL) was added, and a precipitate formed. The precipitate was isolated by filtration, washed with dichloromethane, and dried overnight in a vacuum oven at 65° C. to provide 0.571 g of N-{[4-amino-1-(2-methylpropyl)-1H-imidazo[4,5-c]quinolin-2-yl]methyl}-N-methylurea as white crystals, mp 223-225° C.

¹H NMR (300 MHz, DMSO-d₆) δ 8.01 (d, J=7.3 Hz, 1H), 7.62 (dd, J=8.4, 1.2 Hz, 1H), 7.43 (ddd, J=8.3, 7.0, 1.2 Hz, 1H), 7.26 (ddd, J=8.3, 7.0, 1.2 Hz, 1H), 6.56 (m, 1H) 6.51 (br s, 2H), 5.94 (q, J=4.7 Hz, 1H), 4.57 (d, J=5.6 Hz, 2H), 4.43 (d, J=7.6 Hz, 2H), 2.60 (d, J=4.7 Hz, 3H), 2.19 (m, 1H), 0.92 (d, J=6.7 Hz, 6H);

MS (APCD m/z 327.1 (M+H)⁺;

Anal. Calcd for C₁₇H₂₂N₆O: C, 62.56; H, 6.79; N, 25.75. Found: C, 62.30; H, 6.94; N, 25.68.

### Example 2

### N-{[4-Amino-1-(2-methylpropyl)-1H-imidazo[4,5-c]quinolin-2-yl]methyl}-N′-ethylurea

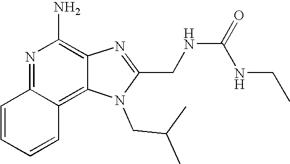

Ethyl isocyanate (0.323 mL, 4.08 mmol) was added to a stirred suspension of 2-(aminomethyl)-1-(2-methylpropyl)-1H-imidazo[4,5-c]quinolin-4-amine (1.0 g, 3.7 mmol) in DMF (10 mL), and the resulting solution was stirred at ambient temperature for 30 minutes. A precipitate formed and was isolated by filtration, washed with dichloromethane, and dried overnight in a vacuum oven at 65° C. to provide 0.511 g of N-{[4-amino-1-(2-methylpropyl)-1H-imidazo[4,5-c]quinolin-2-yl]methyl}-m-ethylurea as white crystals, mp 225-227° C.

¹H NMR (300 MHz, DMSO-d₆) 8.00 (d, J=8.4 Hz, 1H), 7.62 (dd, J=8.3, 1.2 Hz, 1H), 7.43 (ddd, J=8.3, 7.0, 1.2 Hz, 1H), 7.26 (ddd, J=8.3, 7.0, 1.2 Hz, 1H), 6.52 (br s, 2H), 6.48 (m, 1H), 6.02 (t, J=5.5 Hz, 1H), 4.57 (d, J=5.8 Hz, 2H), 4.43 (d, J=7.5 Hz, 2H), 3.06 (dq, J=7.2, 5.6 Hz, 2H), 2.20 (m, 1H), 1.00 (t, J=7.2 Hz, 3H), 0.92 (d, J=6.7 Hz, 6H);

MS (APCI) m/z 341.1 (M+H)⁺;

Anal. Calcd for C₁₈H₂₄N₆O: C, 63.51; H, 7.11; N, 24.69. Found: C, 63.20; H, 6.94; N, 24.71.

### Example 3

### N′-{[4-Amino-1-(2-methylpropyl)-1H-imidazo[4,5-c]quinolin-2-yl]methyl}-N,N-dimethylurea

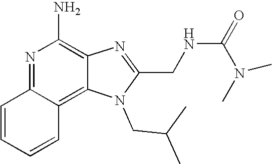

Triethylamine (0.776 mL, 5.57 mmol) and dimethylcarbamyl chloride (0.376 mL, 4.08 mmol) were sequentially added to a stirred suspension of 2-(aminomethyl)-1-(2-methylpropyl)-1H-imidazo[4,5-c]quinolin-4-amine (1.0 g, 3.7 mmol) in DMF (10 mL). After the reaction mixture was stirred at ambient temperature for one hour, an analysis by LC/MS indicated the presence of starting material. Additional triethylamine (0.300 mL) and dimethylcarbamyl chloride (0.200 mL) were added, and the resulting solution was stirred at ambient temperature for 45 minutes. The solution was diluted with dichloromethane (30 mL) and washed with saturated aqueous sodium bicarbonate (1×50 mL). The aqueous layer was extracted with dichloromethane (3×50 mL), and the combined organic fractions were allowed to stand overnight and then concentrated under reduced pressure to provide 1.36 g of the crude product as a light yellow solid. The crude product was dissolved in dichloromethane (15 mL) and purified by prep HPLC on a HORIZON HPFC system using a FLASH 40+M cartridge (eluting with chloroform:methanol in a gradient from 95:5 to 85:15). The fractions containing the desired product were combined and concentrated under reduced pressure, and the resulting solid was dried overnight in a vacuum oven at 65° C. to provide 0.323 g of N-{[4-amino-1-(2-methylpropyl)-1H-imidazo[4,5-c]quinolin-2-yl]methyl}-N,N-dimethylurea as white crystals, mp 162-163° C.

¹H NMR (300 MHz, DMSO-d₆) δ 8.00 (d, J=8.1 Hz, 1H), 7.62 (dd, J=8.4, 1.2 Hz, 1H), 7.42 (ddd, J=8.3, 7.0, 1.2 Hz, 1H), 7.26 (ddd, J=8.3, 7.0, 1.2 Hz, 1H), 6.96 (t, J=5.6 Hz, 1H) 6.50 (br s, 2H), 4.59 (d, J=5.5 Hz, 2H), 4.47 (d, J=7.6 Hz, 2H), 2.83 (s, 6H), 2.20 (m, 1H), 0.92 (d, J=6.7 Hz, 6H);

MS (APCI) m/z 341.1 (M+H)⁺;

Anal. Calcd for C₁₈H₂₄N₆O: C, 63.51; H, 7.11; N, 24.69. Found: C, 63.28; H, 7.22; N, 24.48.

### Examples 4-27

An isocyanate, isothiocyanate, or carbamoyl chloride (0.09 mmol, 0.9 equivalents) from the table below was added to a test tube containing 2-(aminomethyl)-1-(2-methylpropyl)-1H-imidazo[4,5-c]quinolin-4-amine (27 mg, 0.10 mmol) and N,N-diisopropylethylamine (0.022 mL, 0.12 mmol) in DMF (2 mL). The test tube was capped and shaken overnight at ambient temperature. One drop of water was added to each test tube, and the solvent was removed by vacuum centrifugation.

The compounds were purified by reversed phase prep HPLC using a Waters Fraction Lynx automated purification system. The prep HPLC fractions were analyzed using a Micromass LC/TOF-MS, and the appropriate fractions were centrifuge evaporated to provide the trifluoroacetate salt of the desired compound. Column: Zorbax BonusRP, 21.2×50 millimeters (mm), 5 micron particle size; non-linear gradient elution from 5-95% B where A is 0.05% trifluoroacetic acid/water and B is 0.05% trifluoroacetic acid/acetonitrile; fraction collection by mass-selective triggering. The table below shows the reagent (isocyanate, isothiocyanate, or carbamoyl chloride) used for each example, the structure of the resulting compound, and the observed accurate mass for the isolated trifluoroacetate salt.

### Examples 4-27

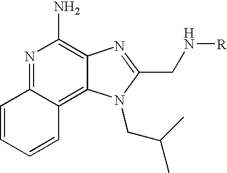

| | | | Measured Mass |
|---|---|---|---|
| Example | Reagent | R | (M + H) |
| 4 | n-Propyl isocyanate | 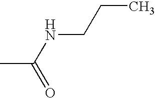 | 355.2248 |
| 5 | Cyclopropyl isothiocyanate | 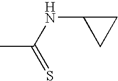 | 369.1892 |
| 6 | Dimethylcarbamyl chloride | 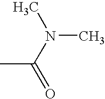 | 341.2094 |
| 7 | Benzyl isocyanate | 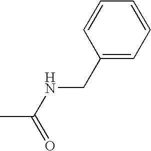 | 403.2267 |
| 8 | m-Tolyl isocyanate | 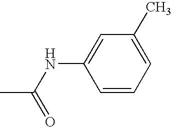 | 403.2275 |
| 9 | p-Tolyl isocyanate | 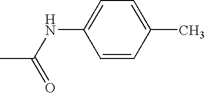 | 403.2258 |
| 10 | 2-Fluorophenyl isocyanate | 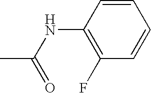 | 407.2033 |
| 11 | 3-Fluorophenyl isocyanate | 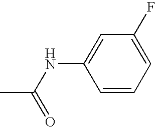 | 407.2017 |
| 12 | 3-Cyanophenyl isocyanate | 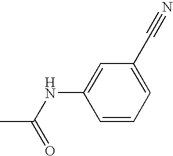 | 414.2073 |
| 13 | 4-Cyanophenyl isocyanate | 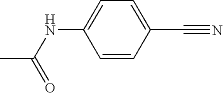 | 414.2049 |
| 14 | Benzoyl isocyanate | 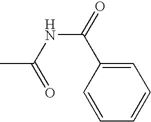 | 417.2049 |
| 15 | Phenethyl isocyanate | 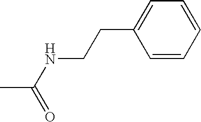 | 417.2402 |
| 16 | 3-Methoxyphenyl isocyanate | 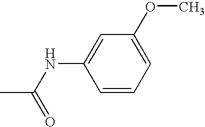 | 419.2209 |
| 17 | 4-Methoxyphenyl isocyanate | 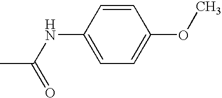 | 419.2217 |
| 18 | Morpholine-4-carbonyl chloride | 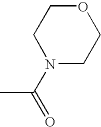 | 383.2227 |
| 19 | 2-Chlorophenyl isocyanate | 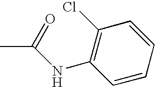 | 423.1710 |
| 20 | trans-2-Phenylcyclopropyl isocyanate | 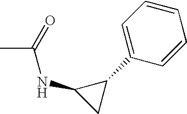 | 429.2434 |
| 21 | 3-Cyanophenyl isothiocyanate | 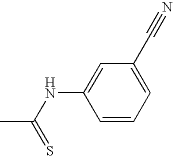 | 430.1816 |
| 22 | 3-Acetylphenyl isocyanate | 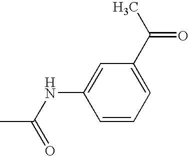 | 431.2216 |
| 23 | 4-(Dimethylamino)phenyl isocyanate | 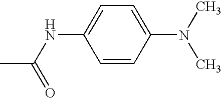 | 432.2502 |
| 24 | 3-Nitrophenyl isocyanate | 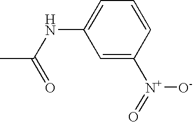 | 434.1958 |
| 25 | 3-(Diethylamino)propyl isothiocyanate | 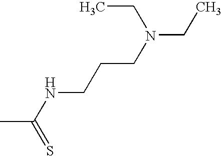 | 442.2769 |
| 26 | 3-(Trifluoromethyl)phenyl isocyanate | 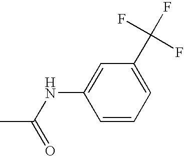 | 457.1966 |
| 27 | 4-(Trifluoromethyl)phenyl isocyanate | 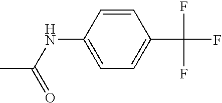 | 457.1960 |

### Examples 28-40

### Part A

Under a nitrogen atmosphere, a solution of 2-(chloromethyl)-1-(2-methylpropyl)-1H-imidazo[4,5-c]quinolin-4-amine (2.0 g, 6.9 mmol), piperazine (6 g, 70 mmol), and N,N-diisopropylethylamine (1.4 mL, 14 mmol) in acetonitrile (100 mL) was heated at reflux for three hours, cooled to 60° C., and stirred overnight. The solvent was removed under reduced pressure, and the residue was dissolved in chloroform. The resulting solution was washed with water (4×100 mL) and concentrated under reduced pressure to provide 1.7 g of 1-(2-methylpropyl)-2-(piperazin-1-ylmethyl)-1H-imidazo[4,5-c]quinolin-4-amine.

### Part B

An isocyanate (0.110-0.120 mmol, 0.11-0.125 equivalents) from the table below was added to a test tube containing 1-(2-methylpropyl)-2-(piperazin-1-ylmethyl)-1H-imidazo[4,5-c]quinolin-4-amine (32.6 mg, 0.096 mmol) and N,N-diisopropylethylamine (0.022 mL, 0.126 mmol) in chloroform (2 mL). The test tube was capped, shaken for four hours at ambient temperature, and allowed to stand at ambient temperature overnight. The solvent was removed by vacuum centrifugation, and the compounds were purified by prep HPLC according to the method described in Examples 4-27. The table below shows the isocyanate added to each test tube, the structure of the resulting compound, and the observed accurate mass for the isolated trifluoroacetate salt.

### Examples 28-40

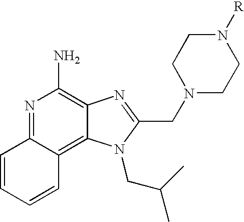

| | | | Measured Mass |
|---|---|---|---|
| Example | Isocyanate | R | (M + H) |
| 28 | Isopropyl isocyanate | 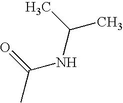 | 424.2817 |
| 29 | tert-Butyl isocyanate | 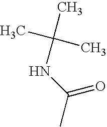 | 438.2981 |
| 30 | Phenyl isocyanate | 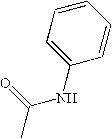 | 458.2675 |
| 31 | Ethyl isocyanatoacetate | 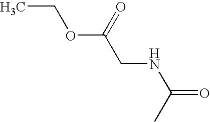 | 468.2721 |
| 32 | Benzyl isocyanate | 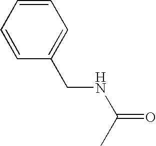 | 472.2815 |
| 33 | m-Tolyl isocyanate | 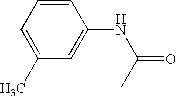 | 472.2844 |
| 34 | o-Tolyl isocyanate | 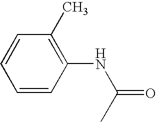 | 472.2798 |
| 35 | p-Tolyl isocyanate | 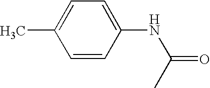 | 472.2812 |
| 36 | 2-Fluorophenyl isocyanate | 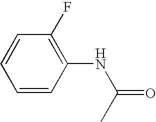 | 476.2566 |
| 37 | 3-Fluorophenyl isocyanate | 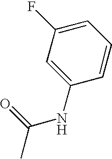 | 476.2572 |
| 38 | 2-Methoxyphenyl isocyanate | 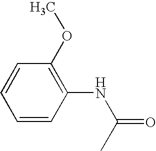 | 488.2740 |
| 39 | 3-Methoxyphenyl isocyanate | 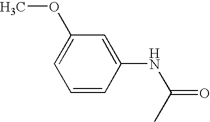 | 488.2749 |
| 40 | 4-Methoxyphenyl isocyanate | 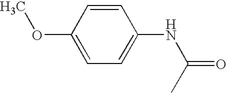 | 488.2779 |

### Example 41

### N-{[4-Amino-1-(2-methylpropyl)-6,7-dimethyl-1H-imidazo[4,5-c]pyridin-2-yl]methyl}-N′-methylurea

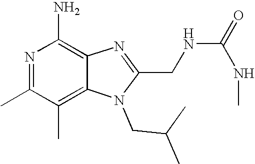

### Part A

Ethyl chloroacetimidate hydrochloride (60 g, 380 mmol), prepared according to the procedure of Stillings, M. R. et al., J. Med. Chem., 29, pp. 2280-2284, (1986), was added to a solution of 5,6-dimethyl-N-(2-methylpropyl)-2-phenoxypyridine-3,4-diamine (36.08 g, 126.4 mmol, see the methods in the examples of U.S. Pat. No. 6,743,920) in chloroform (520 mL), and the reaction was stirred at 60° C. overnight, allowed to cool to ambient temperature, and diluted with chloroform (400 mL). The resulting solution was washed with brine (2×500 mL), dried over magnesium sulfate, filtered through a layer of CELITE filter agent, and concentrated under reduced pressure to provide 53.17 g of a dark brown oil. The oil was purified in two portions by column chromatography on silica gel (eluting with dichloromethane:methanol in a gradient from 99.5:0.5 to 98:2) to provide 18.10 g of 2-(chloromethyl)-6,7-dimethyl-1-(2-methylpropyl)-4-phenoxy-1H-imidazo[4,5-c]pyridine as a light pink solid.

### Part B

A solution of 2-(chloromethyl)-6,7-dimethyl-1-(2-methylpropyl)-4-phenoxy-1H-imidazo[4,5-c]pyridine (8.51 g, 24.7 mmol) and ammonia (300 mL of 7 N solution in methanol) was heated in a high-pressure vessel overnight at 150° C., allowed to cool to ambient temperature, and concentrated under reduced pressure to provide 9.05 g of a dark brown solid. The solid was mixed with 10.53 g of material from another run and purified by column chromatography on silica gel (eluting with dichloromethane:methanol:ammonium hydroxide in a gradient from 89.1:9.9:1 to 85.1:13.9:1) to provide 6.39 g of 2-(aminomethyl)-6,7-dimethyl-1-(2-methylpropyl)-1H-imidazo[4,5-c]pyridin-4-amine as a brown solid.

### Part C

Triethylamine (0.880 mL, 6.31 mmol) and methyl isocyanate (0.270 mg, 4.73 mmol) were sequentially added to a solution of 2-(aminomethyl)-6,7-dimethyl-1-(2-methylpropyl)-1H-imidazo[4,5-c]pyridin-4-amine (0.780 g, 3.15 mmol) in dichloromethane (20 mL), and the reaction was stirred at ambient temperature for 1.5 hours. The solution was then diluted with dichloromethane (20 mL) and washed with brine (4×35 mL). The combined aqueous washings were extracted with dichloromethane (1×40 mL), and the combined organic fractions were dried over magnesium sulfate, filtered through a layer of CELITE filter agent, and concentrated under reduced pressure to provide 440 mg of the crude product as a light brown solid. The crude product was purified twice by column chromatography on silica gel (eluting first with 94:5:1 dichloromethane:methanol:ammonium hydroxide and second with dichloromethane:methanol:ammonium hydroxide in a gradient from 97:2:1 to 94:5:1) to provide 110 mg of N-{[4-amino-1-(2-methylpropyl)-6,7-dimethyl-1H-imidazo[4,5-c]pyridin-2-yl]methyl}-v-methylurea as a beige powder, mp 205-206° C.

Anal. Calcd for C₁₅H₂₄N₆O.0.2CH₂Cl₂: C, 56.81; H, 7.65; N, 26.15. Found: C, 56.69; H, 8.18; N, 25.79.

MS (APCI) m/z 305.2088 (M+H)⁺.

### Examples 42-53

An isocyanate, isothiocyanate, or carbamoyl chloride (0.12 mmol, 1.2 equivalents) from the table below was added to a test tube containing 2-(aminomethyl)-6,7-dimethyl-1-(2-methylpropyl)-1H-imidazo[4,5-c]pyridin-4-amine (24.3 mg, 0.098 mmol) and N,N-diisopropylethylamine (0.057 mL, 0.33 mmol) in DMF (1 mL). The test tube was capped and shaken overnight at ambient temperature, and then the solvent was removed by vacuum centrifugation. The compounds were purified by prep HPLC according to the method described in Examples 4-27. The table below shows the reagent (isocyanate, isothiocyanate, or carbamoyl chloride) added to each test tube, the structure of the resulting compound, and the observed accurate mass for the isolated trifluoroacetate salt.

### Examples 42-53

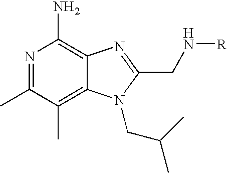

| | | | Measured Mass |
|---|---|---|---|
| Example | Reagent | R | (M + H) |
| 42 | Methyl isocyanate | 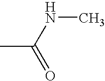 | 305.2085 |
| 43 | Ethyl isocyanate | 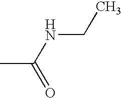 | 319.2277 |
| 44 | Methyl isothiocyanate | 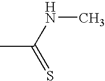 | 321.1877 |
| 45 | Ethyl isothiocyanate | 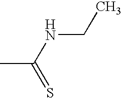 | 335.2043 |
| 46 | Cyclopropyl isothiocyanate | 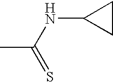 | 347.2019 |
| 47 | Isopropyl isothiocyanate | 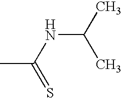 | 349.2180 |
| 48 | Phenyl isocyanate | 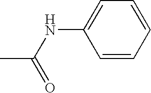 | 367.2251 |
| 49 | 3-Pyridyl isothiocyanate | 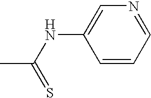 | 384.1998 |
| 50 | (R)-(+)-alpha-Methylbenzyl isocyanate | 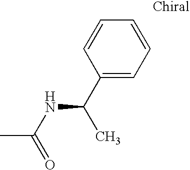 | 395.2566 |
| 51 | 3,4-Difluorophenyl isocyanate | 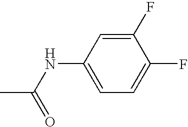 | 403.2092 |
| 52 | N,N-Dimethylcarbamoyl chloride | 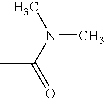 | 319.2251 |
| 53 | N-Methyl-N- phenylcarbamoyl chloride | 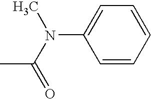 | 381.2421 |

### Examples 54-65

### Part A

A solution of N⁴-(2-methylpropyl)[1,5]naphthyridine-3,4-diamine (approximately 15 g, 70 mmol, U.S. Pat. No. 6,194,425 Example 30, Part A), dichloromethane (280 mL) was cooled to 0° C.; chloroacetyl chloride (6.1 mL, 77 mmol) was added dropwise over a period of ten minutes. The reaction was allowed to warm to ambient temperature, stirred for two hours, and concentrated under reduced pressure to provide 2-chloro-N⁴-(2-methylpropylamino)-([1,5]naphthyridin-3-yl)acetamide hydrochloride as a pale-yellow solid.

### Part B

Aqueous potassium carbonate (17.5 mL of 6 M, 105 mmol) was added to a solution of the material from Part A in 3:1 ethanol:water (280 mL); the reaction was stirred for three days and concentrated under reduced pressure. The residue was partitioned between dichloromethane (200 mL) and brine (100 mL). The aqueous layer was separated and extracted with dichloromethane (2×50 mL). The combined organic fractions were dried over magnesium sulfate, filtered, and concentrated under reduced pressure to provide 19.5 g of 2-(chloromethyl)-1-(2-methylpropyl)-1H-imidazo[4,5-c][1,5]naphthyridine as a brown solid containing a small amount of dichloromethane.

### Part C

3-Chloroperoxybenzoic acid (5.38 g of 77% pure material, 31.2 mmol) was added to a solution of 2-(chloromethyl)-1-(2-methylpropyl)-1H-imidazo[4,5-c][1,5]naphthyridine (3.0 g, 11 mmol) in chloroform (45 mL); the reaction mixture was stirred at ambient temperature for one hour. An analysis by LC/MS indicated the reaction was incomplete, and additional 3-chloroperoxybenzoic acid (1.8 g) was added. The reaction was stirred for one hour and diluted with dichloromethane (150 mL) and saturated aqueous sodium bicarbonate (75 mL). The organic layer was separated and washed with saturated aqueous sodium bicarbonate (75 mL). The combined aqueous fractions were extracted with dichloromethane (2×30 mL), and the combined organic layers were dried over magnesium sulfate, filtered, and concentrated under reduced pressure to provide 2-(chloromethyl)-1-(2-methylpropyl)-5-oxido-1H-imidazo[4,5-c][1,5]naphthyridine as an orange semi-solid.

### Part D

A solution of the material from Part C in methanol (40 mL) was cooled to 0° C., and ammonium hydroxide (3.6 mL of 15 M) was added. Benzenesulfonyl chloride (2.9 mL, 23 mmol) was added dropwise over a period of ten minutes, and the reaction was stirred at 0° C. for one hour and then concentrated under reduced pressure. The residue was partitioned between dichloromethane (120 mL) and saturated aqueous sodium bicarbonate (80 mL). The aqueous layer was extracted with dichloromethane (2×25 mL), and the combined organic fractions were dried over magnesium sulfate, filtered, and concentrated under reduced pressure. The resulting brown solid was triturated with chloroform, isolated by filtration, and purified by prep HPLC on a HORIZON HPFC system using a FLASH 40+M cartridge (eluting with chloroform:CMA in a gradient from 100:0 to 75:25) to provide 1.82 g of 2-(chloromethyl)-1-(2-methylpropyl)-1H-imidazo[4,5-c][1,5]naphthyridin-4-amine as a yellow solid.

### Part E

Potassium phthalimide (1.40 g, 7.54 mmol) was added to a solution of 2-(chloromethyl)-1-(2-methylpropyl)-1H-imidazo[4,5-c][1,5]naphthyridin-4-amine (1.82 g, 6.28 mmol) in DMF (50 mL). The reaction mixture was stirred at ambient temperature for three hours, and a white precipitate formed. The DMF was removed under reduced pressure, and the residue was triturated with methanol, isolated by filtration, and dried under high vacuum to provide 1.51 g of 2-{[4-amino-1-(2-methylpropyl)-1H-imidazo[4,5-c][1,5]naphthyridin-2-yl]methyl}-1H-isoindole-1,3(2H)-dione.

### Part F

Hydrazine (0.59 mL, 19 mmol) was added to a stirred suspension of 2-{[4-amino-1-(2-methylpropyl)-1H-imidazo[4,5-c][1,5]naphthyridin-2-yl]methyl}-1H-isoindole-1,3(2H)-dione (1.51 g, 3.77 mmol) in ethanol (60 mL). After four hours at ambient temperature, an analysis by HPLC indicated the presence of starting material. Additional hydrazine (0.3 mL) was added, and the reaction was stirred at ambient temperature overnight. The ethanol was removed under reduced pressure, and the residue was sonicated in hydrochloric acid (30 mL of 1 M) for 15 minutes. The resulting mixture was filtered to remove a solid, which was washed with water. The filtrate was adjusted to pH 7 with the addition of solid sodium bicarbonate. A white precipitate formed and was isolated by filtration, washed with water, and dried for three hours in a vacuum oven at 60° C. to provide 1.02 g of 2-(aminomethyl)-1-(2-methylpropyl)-1H-imidazo[4,5-c][1,5]naphthyridin-4-amine.

### Part G

A reagent (0.11 mmol, 1.1 equivalents) from the table below was added to a test tube containing 2-(aminomethyl)-1-(2-methylpropyl)-1H-imidazo[4,5-c][1,5]naphthyridin-4-amine (27 mg, 0.10 mmol) and N,N-diisopropylethylamine (0.035 mL, 0.20 mmol) in DMF (1 mL). The test tube was capped and shaken overnight at ambient temperature. Two drops of water were added to each test tube, and the solvent was removed by vacuum centrifugation.

The compounds were purified by prep HPLC according to the method described in Examples 4-27. The table below shows the reagent added to each test tube, the structure of the resulting compound, and the observed accurate mass for the isolated trifluoroacetate salt.

### Examples 54-65

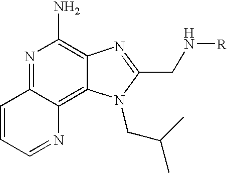

| | | | Measured Mass |
|---|---|---|---|
| Example | Reagent | R | (M + H) |
| 54 | Methyl chloroformate | 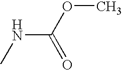 | 329.1715 |
| 55 | Ethyl chloroformate | 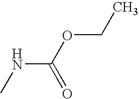 | 343.1884 |
| 56 | Methyl isocyanate | 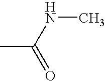 | 328.1895 |
| 57 | Ethyl isocyanate | 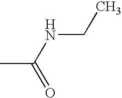 | 342.2015 |
| 58 | Isopropyl isocyanate | 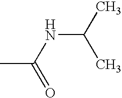 | 356.2182 |
| 59 | Isopropyl isothiocyanate | 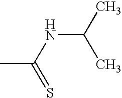 | 372.1953 |
| 60 | Phenyl isocyanate | 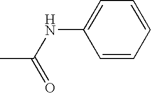 | 390.2025 |
| 61 | Benzylisocyanate | 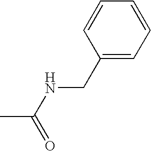 | 404.2169 |
| 62 | Phenethyl isocyanate | 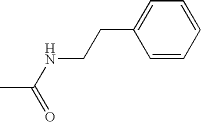 | 418.2346 |
| 63 | 3,4-Difluorophenyl isocyanate | 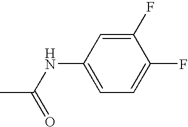 | 426.1846 |
| 64 | 3,4-Dimethoxyphenyl isocyanate | 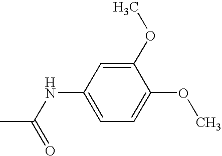 | 450.2234 |
| 65 | N,N-Dimethylcarbamoyl chloride | 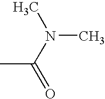 | 342.2022 |

### Example 66-115

### Part A

Triethylamine (58.2 g, 575 mmol) and 4-chloro-3-nitroquinoline (80.0 g, 384 mmol) were added to a solution of tert-butyl N-(2-aminoethyl)carbamate (67.6 g, 422 mmol) in DMF (300 mL), and the reaction was stirred overnight at ambient temperature. Water (600 mL) was added, and the resulting mixture was stirred for one hour. A precipitate formed and was isolated by filtration, washed with water (3×150 mL), and dried for two days in a vacuum oven at 45° C. to provide 125.36 g of tert-butyl 2-[(3-nitroquinolin-4-yl)amino]ethylcarbamate as a yellow solid.

### Part B

A solution of tert-butyl 2-[(3-nitroquinolin-4-yl)amino]ethylcarbamate (46.46 g, 139.8 mmol) in ethyl acetate was added to a Parr vessel; 5% platinum on carbon (16.4 g, 84.0 mmol) was added. The vessel was placed under hydrogen pressure (3.0 psi, 2.1×10⁵ Pa) and shaken overnight. The reaction mixture was filtered through a layer of CELITE filter agent, and the filter cake was washed with methanol and dichloromethane. The filtrate was concentrated under reduced pressure to provide 40.23 g of tert-butyl 2-[(3-aminoquinolin-4-yl)amino]ethylcarbamate.

### Part C

Triethylamine (37.1 mL, 266 mmol) and chloroacetyl chloride (10.6 mL, 133 mmol) were sequentially added to a solution of tert-butyl 2-[(3-aminoquinolin-4-yl)amino]ethylcarbamate (40.23 g, 133 mmol) in dichloromethane (400 mL), and the reaction was stirred at ambient temperature for ten minutes and then concentrated under reduced pressure. The residue was further dried under high vacuum for 30 minutes and then dissolved in ethanol (1 L). The resulting solution was stirred for two days at ambient temperature and concentrated under reduced pressure. The residue was dissolved in dichloromethane, and the resulting solution was washed sequentially with 5% aqueous ammonium chloride and water, dried over magnesium sulfate, filtered, concentrated under reduced pressure, and further dried under high vacuum to provide 50.73 g of tert-butyl 2-[2-(chloromethyl)-1H-imidazo[4,5-c]quinolin-1-yl]ethylcarbamate.

### Part D

3-Chloroperoxybenzoic acid (7.5 g of 77% pure material, 33 mmol) was added to a solution of tert-butyl 2-[2-(chloromethyl)-1H-imidazo[4,5-c]quinolin-1-yl]ethylcarbamate (10.0 g, 27.7 mmol) in chloroform; the reaction mixture was stirred at ambient temperature for one hour. Additional portions of 3-chloroperoxybenzoic acid were added, and the reaction was stirred until analysis by thin layer chromatography (TLC) indicated that the reaction was complete. Ammonium hydroxide (100 mL) and p-toluenesulfonyl chloride (5.81 g, 30.45 mmol) were sequentially added, and the reaction mixture was stirred vigorously at ambient temperature overnight. The organic layer was separated, washed with ammonium hydroxide, and concentrated under reduced pressure. The crude product was purified by normal phase prepHPLC (eluting with dichloromethane:methanol:triethylamine in a gradient from 100:0:0 to 95:4.5:0.5) to provide 3.99 g of tert-butyl 2-[4-amino-2-(chloromethyl)-1H-imidazo[4,5-c]quinolin-1-yl]ethylcarbamate.

### Part E

A solution of tert-butyl 2-[4-amino-2-(chloromethyl)-1H-imidazo[4,5-c]quinolin-1-yl]ethylcarbamate (3.99 g, 10.6 mmol) and ammonia (50 mL of 7 N solution in methanol) was stirred overnight at ambient temperature, concentrated under reduced pressure, and further dried under high vacuum to provide 3.49 g of tert-butyl 2-[4-amino-2-(aminomethyl)-1H-imidazo[4,5-c]quinolin-1-yl]ethylcarbamate.

### Part F

Methyl isocyanate (610.6 mg, 10.70 mmol) was added to a solution of tert-butyl 2-[4-amino-2-(aminomethyl)-1H-imidazo[4,5-c]quinolin-1-yl]ethylcarbamate (3.47 g, 9.73 mmol) in DMF (35 mL), and the resulting solution was stirred at ambient temperature for two days. The solvent was removed under reduced pressure, and the residue was purified by normal phase prepHPLC (eluting with dichloromethane:methanol:triethylamine in a gradient from 100:0:0 to 90:9:1) to provide 2.2 g of tert-butyl 2-[4-amino-2-({[(methylamino)carbonyl]amino}methyl)-1H-imidazo[4,5-c]quinolin-1-yl]ethylcarbamate.

### Part G

Hydrogen chloride (20 mL of a 4 N solution in 1,4-dioxane) was added to a solution of tert-butyl 2-[4-amino-2-({[(methylamino)carbonyl]amino}methyl)-1H-imidazo[4,5-c]quinolin-1-yl]ethylcarbamate (2.2 g, 5.3 mmol) in dichloromethane (40 mL) and methanol (5 mL), and the reaction was stirred overnight at ambient temperature. Diethyl ether was added to the reaction, and a precipitate formed. The precipitate was isolated by filtration, washed with diethyl ether, and dissolved in methanol. An excess of triethylamine was added, and the resulting mixture was concentrated under reduced pressure. The residue was washed with dichloromethane to provide 1.72 g of N-{[4-amino-1-(2-aminoethyl)-1H-imidazo[4,5-c]quinolin-2-yl]methyl}-N′-methylurea.

### Part H

A reagent (0.11 mmol, 1.1 equivalents) from the table below was added to a test tube containing N-{[4-amino-1-(2-aminoethyl)-1H-imidazo[4,5-c]quinolin-2-yl]methyl}-N-methylurea (31 mg, 0.10 mmol) and N,N-diisopropylethylamine (0.034 mL, 0.20 mmol) in DMF (1 mL). The test tube was capped and shaken overnight at ambient temperature. Two drops of water were added to each test tube, and the solvent was removed by vacuum centrifugation.

The compounds were purified by prep HPLC according to the method described in Examples 4-27. The table below shows the reagent added to each test tube, the structure of the resulting compound, and the observed accurate mass for the isolated trifluoroacetate salt.

### Examples 66-115

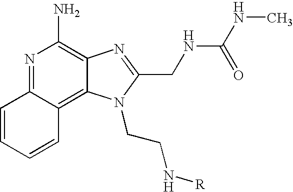

| | | | Measured Mass |
|---|---|---|---|
| Example | Reagent | R | (M + H) |
| 66 | none | 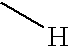 | 314.1711 |
| 67 | Acetyl chloride | 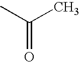 | 356.1843 |
| 68 | Methyl chloroformate | 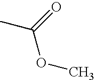 | 372.1799 |
| 69 | Cyclopropanecarbonyl chloride | 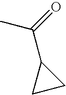 | 382.1977 |
| 70 | Methyl chlorothiolformate | 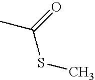 | 388.1542 |
| 71 | tert-Butylacetyl chloride | 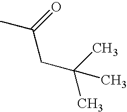 | 412.2461 |
| 72 | Benzoyl chloride | 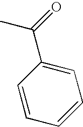 | 418.1974 |
| 73 | Cyclopentylacetyl chloride | 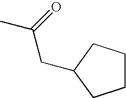 | 424.2456 |
| 74 | m-Toluoyl chloride | 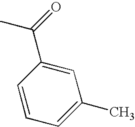 | 432.2137 |
| 75 | 3-Fluorobenzoyl chloride | 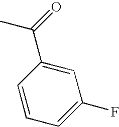 | 436.1890 |
| 76 | 3-Cyanobenzoyl chloride | 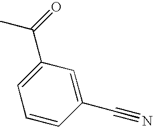 | 443.1948 |
| 77 | 3-Methoxybenzoyl chloride | 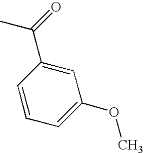 | 448.2080 |
| 78 | 3-Chlorobenzoyl chloride | 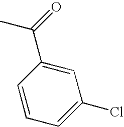 | 452.1571 |
| 79 | Isonicotinoyl chloride hydrochloride | 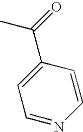 | 419.1941 |
| 80 | Nicotinoyl chloride hydrochloride | 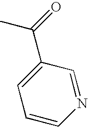 | 419.1918 |
| 81 | trans-2-Phenyl-1- cyclopropanecarbonyl chloride | 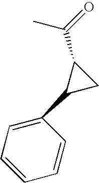 | 458.2301 |
| 82 | 2-Naphthoyl chloride | 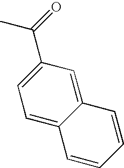 | 468.2154 |
| 83 | 3-Indoleglyoxylyl chloride | 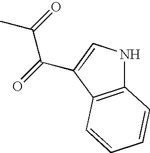 | 485.2027 |
| 84 | 4-Biphenylcarbonyl chloride | 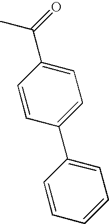 | 494.2285 |
| 85 | Methanesulfonyl chloride | 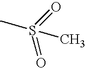 | 392.1501 |
| 86 | Isopropylsulfonyl chloride | 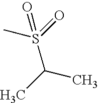 | 420.1808 |
| 87 | Dimethylsulfamoyl chloride | 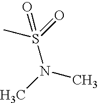 | 421.1761 |
| 88 | Trifluoromethanesulfonyl chloride | 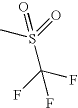 | 446.1208 |
| 89 | Benzenesulfonyl chloride | 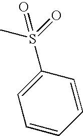 | 454.1667 |
| 90 | 1-Methylimidazole-4- sulfonyl chloride | 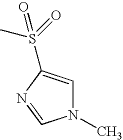 | 458.1718 |
| 91 | 2-Thiophenesulfonyl chloride | 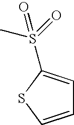 | 460.1195 |
| 92 | α-Toluenesulfonyl chloride | 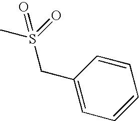 | 468.1798 |
| 93 | 3-Fluorobenzenesulfonyl chloride | 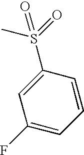 | 472.1556 |
| 94 | β-Styrenesulfonyl chloride | 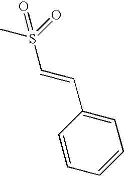 | 480.1786 |
| 95 | 3-Methoxybenzenesulfonyl chloride | 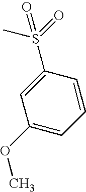 | 484.1732 |
| 96 | 3-Chlorobenzenesulfonyl chloride | 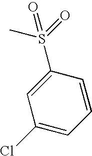 | 488.1230 |
| 97 | 2-Naphthalenesulfonyl chloride | 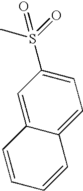 | 504.1810 |
| 98 | 3-(Trifluoromethyl)benzene- sulfonyl chloride | 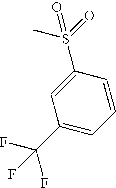 | 522.1498 |
| 99 | 3,4-Dichlorobenzenesulfonyl chloride | 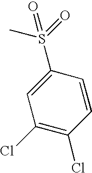 | 522.0850 |
| 100 | Ethyl isocyanate | 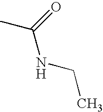 | 385.2105 |
| 101 | Isopropyl isocyanate | 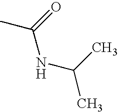 | 399.2246 |
| 102 | Isopropyl isothiocyanate | 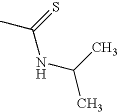 | 415.2017 |
| 103 | Cyclopropylmethyl isothiocyanate | 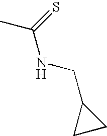 | 427.2034 |
| 104 | Isobutyl isothiocyanate | 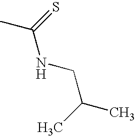 | 429.2170 |
| 105 | Phenyl isocyanate | 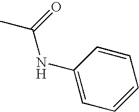 | 433.2098 |
| 106 | Benzyl isocyanate | 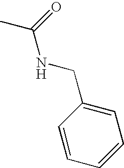 | 447.2237 |
| 107 | 3-Pyridyl isothiocyanate | 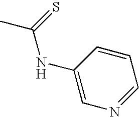 | 450.1799 |
| 108 | Benzoyl isocyanate | 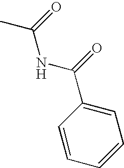 | 461.2035 |
| 109 | 3-Methoxyphenyl isocyanate | 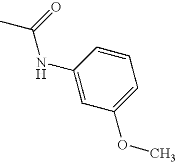 | 463.2194 |
| 110 | 3-Chlorophenyl isocyanate | 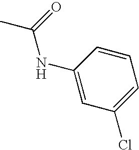 | 467.1705 |
| 111 | 1-Naphthyl isocyanate | 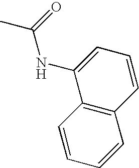 | 483.2242 |
| 112 | 2-Morpholinoethyl isothiocyanate | 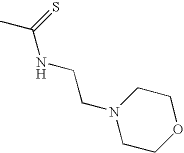 | 486.2375 |
| 113 | N,N-Dimethylcarbonyl chloride | 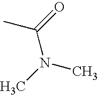 | 385.2067 |
| 114 | 4-Morpholinylcarbonyl chloride | 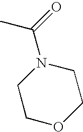 | 427.2167 |
| 115 | N-Methyl-N- phenylcarbamoyl chloride | 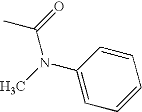 | 447.2228 |

### Example 116

### tert-Butyl 4-[4-amino-1-(2-methylpropyl)-1H-imidazo[4,5-c]quinolin-2-yl]butylcarbamate

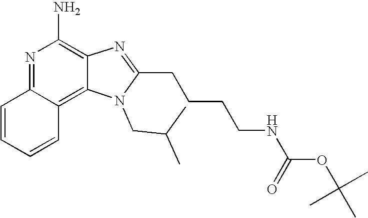

### Part A

Under a nitrogen atmosphere, a solution of 5-[(tertbutoxycarbonyl)amino]pentanoic acid (Boc 5-Ava-OH, 9.50 g, 43.7 mmol) in anhydrous 1,2-dichloroethane (100 mL) was cooled to −20° C., and trimethylacetyl chloride (5.4 mL, 43.7 mmol) and anhydrous triethylamine (25 mL, 0.199 mol) were sequentially added. The reaction was warmed to 0° C. and stirred for three hours. A solution of N⁴-(2-methylpropyl)quinoline-3,4-diamine (8.56 g, 39.8 mmol) in 1,2-dichloroethane (125 mL) was added, and the reaction was allowed to warm to room temperature, heated at reflux overnight, and allowed to cool to room temperature. Chloroform was added, and the resulting solution was washed sequentially with water and cold saturated ammonium chloride (2×200 mL), dried over sodium sulfate, filtered, and concentrated under reduced pressure. The crude product was purified by column chromatography on silica gel (240 g, eluting with 92.5:7.5 dichloromethane:methanol). The column fractions were divided into two portions to provide two solids. Each solid was dissolved in a small volume of dichloromethane, and hexanes were added to cause a precipitate to form. The precipitate was isolated by filtration, and the filtrate was concentrated and treated again with dichloromethane and hexanes as described above. The process was repeated until no additional solid precipitated with the addition of hexanes. A mixture of tert-butyl 4-[1-(2-methylpropyl)-1H-imidazo[4,5-c]quinolin-2-yl]butylcarbamate containing a small amount of tert-butyl 5-({4-[(2-methylpropyl)amino]quinolin-3-yl}amino)-5-oxopentylcarbamate (9.26 g total) was obtained.

### Part B

3-Chloroperoxybenzoic acid (1.60 g of 60% pure material, 5 mmol) was added in one portion to a solution of the material from Part A (1.63 g, 4.11 mmol) in chloroform (50 mL); the reaction mixture was stirred at room temperature overnight. An analysis by TLC indicated the presence of starting material, and additional 3-chloroperoxybenzoic acid (0.40 g) was added. The reaction was stirred for an additional three hours and then washed sequentially with saturated aqueous sodium bicarbonate (2×100 mL) and brine (100 mL), dried over magnesium sulfate, filtered, and concentrated under reduced pressure to provide tert-butyl 4-[1-(2-methylpropyl)-5-oxido-1H-imidazo[4,5-c]quinolin-2-yl]butylcarbamate as an orange solid.

### Part C

Concentrated ammonium hydroxide (10 mL) was added to a stirred solution of the material from Part B in chloroform (50 mL). The mixture was stirred rapidly under a nitrogen atmosphere and cooled to 0° C. p-Toluenesulfonyl chloride (1.57 g, 8.23 mmol) was added in portions over a period of 45 minutes. The reaction mixture was stirred at 0° C. for 15 minutes, allowed to warm to room temperature, and stirred overnight. An analysis by HPLC indicated the presence of starting material, and the reaction was cooled to 0° C. Additional p-toluenesulfonyl chloride (0.79 g) was added, and the reaction mixture was stirred at 0° C. for 15 minutes, allowed to warm to room temperature, and stirred for two hours. The organic layer was separated and washed sequentially with 1% aqueous sodium carbonate (2×50 mL) and water (100 mL), dried over magnesium sulfate, filtered, and concentrated under reduced pressure to provide a sticky, orange solid. The solid was dissolved in a small volume of dichloromethane, and hexanes were added to cause a precipitate to form. The precipitate was isolated by filtration. A second crop of solid was isolated from the mother liquor and washed with hexanes. The two solids were combined to provide 1.62 g of tert-butyl 4-[4-amino-1-(2-methylpropyl)-1H-imidazo[4,5-c]quinolin-2-yl]butylcarbamate as a white crystalline solid, mp 165-167° C.

¹H NMR (300 MHz, DMSO-d₆) δ 7.98 (d, J=7.8 Hz, 1H), 7.61 (dd, J=8.3, 1.0 Hz, 1H), 7.40 (m, 1H), 7.25 (m, 1H), 6.80 (m, 1H), 6.45 (s, 2H), 4.34 (d, J=7.8 Hz, 2H), 3.02-2.88 (m, 4H), 2.17 (m, 1H), 1.80 (m, 2H), 1.54 (m, 2H), 1.37 (s, 9H), 0.93 (d, J=6.8 Hz, 6H);

MS (APCI) m/z 412 (M+H);

Anal calcd for C₂₃H₃₃N₅O₂: C, 67.13; H, 8.08; N, 17.02. Found: C, 67.10; H, 7.93; N, 16.82.

### Example 117

### N-{4-[4-Amino-1-(2-methylpropyl)-6,7,8,9-tetrahydro-1H-imidazo[4,5-c]quinolin-2-yl]butyl}-N′-phenylurea

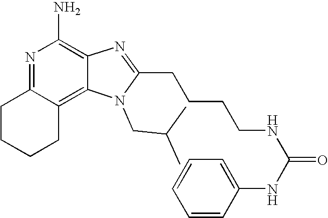

### Part A

A solution of tert-butyl 4-[4-amino-1-(2-methylpropyl)-1H-imidazo[4,5-c]quinolin-2-yl]butylcarbamate (6.81 g, 16.5 mmol) in trifluoroacetic acid (135 mL) was added to a Parr vessel charged with platinum(IV) oxide (0.55 g, 11.2 mmol), and the reaction was placed under hydrogen pressure (50 psi, 3.4×10⁵ Pa). The progress of the reaction was followed by TLC and LC/MS. Additional platinum(IV) oxide was added after three days (0.61 g), after six days (0.50 g), after seven days (0.69 g), and after ten days (0.20 g), and the reaction was placed under hydrogen pressure for a total of two weeks. The reaction mixture was then filtered through a layer of CELITE filter agent, and the filtrate was concentrated under reduced pressure. The residue was dissolved in 6 M hydrochloric acid, and the resulting solution was washed with dichloromethane and then made basic with the addition of 50% w/w aqueous sodium hydroxide. The basic solution was extracted several times with dichloromethane and chloroform. The combined extracts were washed with deionized water, concentrated under reduced pressure, dissolved in toluene, and concentrated under reduced pressure to provide 3.7 g of a light brown solid. The solid was dissolved in hot toluene (600 mL) and filtered. The volume of the solution was reduced to 100 mL and hexanes were added. A precipitate formed, and the mixture was stirred for a few hours before the precipitate was isolated by filtration to provide 3.02 g of 2-(4-aminobutyl)-1-(2-methylpropyl)-6,7,8,9-tetrahydro-1H-imidazo[4,5-c]quinolin-4-amine as a white powder.

### Part B

A solution of 2-(4-aminobutyl)-1-(2-methylpropyl)-6,7,8,9-tetrahydro-1H-imidazo[4,5-c]quinolin-4-amine (1.0 g, 3.2 mmol) in dichloromethane (50 mL) was cooled to 0° C. under a nitrogen atmosphere. Phenyl isocyanate (0.35 mL, 3.2 mmol) was added dropwise, and the reaction was stirred for one hour at 0° C., allowed to warm to room temperature, and stirred overnight. The solvent was removed under reduced pressure, and the residue was purified by column chromatography on silica gel (45 g, eluting with 80:20 dichloromethane:methanol), dried under high vacuum, and farther dried in a vacuum oven overnight at 80° C. to provide 0.60 g of N-{4-[4-amino-1-(2-methylpropyl)-6,7,8,9-tetrahydro-1H-imidazo[4,5-c]quinolin-2-yl]butyl}-N′-phenylurea as an off-white powder, mp 197-199° C.

¹H NMR (300 MHz, DMSO-d₆) δ 8.40 (s, 1H), 7.37 (d, J=7.3 Hz, 2H), 7.20 (m, 2H), 6.87 (t, J=7.3 Hz, 1H), 6.16 (m, 1H), 5.69 (s, 2H), 4.02 (d, J=7.3 Hz, 2H), 3.13 (m, 2H), 2.90 (s, 2H), 2.81 (m, 2H), 2.65 (s, 2H), 1.95 (m, 1H), 1.74 (m, 6H), 1.55 (m, 2H), 0.83 (d, J=6.8 Hz, 6H);

¹³C NMR (125 MHz, DMSO-d₆) 155.1, 153.0, 148.9, 145.7, 140.5, 138.2, 128.5, 124.6, 120.8, 117.5, 105.2, 50.6, 41.1, 32.2, 30.7, 29.4, 26.5, 24.7, 23.3, 22.7, 22.6, 19.1;

MS (ACPI) m/z 435 (M+H);

Anal calcd for C₂₅H₃₄N₆O: C, 69.09; H, 7.89; N, 19.34. Found: C, 68.81; H, 7.69; N, 19.05.

### Example 118

### N-{4-[4-Amino-1-(2-methylpropyl)-1H-imidazo[4,5-c]quinolin-2-yl]butyl}-N′-phenylurea

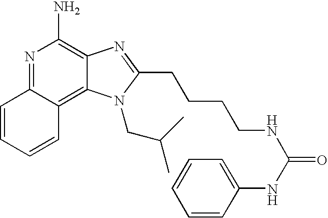

### Part A

Hydrogen chloride (25 mL of a 6 M solution in ethanol) was added to tert-butyl 4-[4-amino-1-(2-methylpropyl)-1H-imidazo[4,5-c]quinolin-2-yl]butylcarbamate (3.60 g, 8.75 mmol), and the resulting solution was diluted with additional ethanol (30 mL). The reaction was heated at reflux for one hour and allowed to cool to room temperature; a precipitate formed as the solution cooled. Nitrogen gas was bubbled through the mixture for one hour. The solvent was removed under reduced pressure, and the residue was dissolved in deionized water and adjusted to pH 11 with the addition of ammonium hydroxide. The basic mixture was extracted with chloroform (2×75 mL), and the combined extracts were concentrated under reduced pressure. Toluene was added to the residue and then removed under reduced pressure to provide 2.38 g of 2-(4-aminobutyl)-1-(2-methylpropyl)-1H-imidazo[4,5-c]quinolin-4-amine.

### Part B

A solution of 2-(4-aminobutyl)-1-(2-methylpropyl)-1H-imidazo[4,5-c]quinolin-4-amine (0.500 g, 1.61 mmol) in dichloromethane (40 mL) was cooled to 0° C. under a nitrogen atmosphere. Phenyl isocyanate (0.178 mL, 1.61 mmol) was added dropwise, and a precipitate formed. The reaction was allowed to warm to room temperature and stirred overnight. The precipitate was isolated by filtration and dried under high vacuum to provide 0.360 g of N-{4-[4-amino-1-(2-methylpropyl)-1H-imidazo[4,5-c]quinolin-2-yl]butyl}-N′-phenylurea as an off-white powder, mp 113-115° C.

¹H NMR (300 MHz, DMSO-d₆) δ 8.42 (s, 1H), 7.98 (d, J=7.6 Hz, 1H), 7.62 (dd, J=8.3, 1.2 Hz, 1H), 7.44-7.37 (m, 3H), 7.29-7.18 (m, 3H), 6.88 (m, 1H), 6.52 (s, 2H), 6.19 (m, 1H), 4.35 (d, J=7.4 Hz, 2H), 3.17 (dd, J=12.4, 6.5 Hz, 2H), 2.95 (t, J=7.5 Hz, 2H), 2.17 (m, 1H), 1.87 (m, 2H), 1.60 (m, 2H), 0.92 (d, J=6.6 Hz, 6H);

¹³C NMR (125 MHz, DMSO-d₆) 155.6, 153.9, 152.0, 144.9, 140.9, 132.7, 129.0, 126.9, 126.6, 126.5, 121.5, 121.3, 120.6, 117.9, 115.2, 51.7, 39.0, 29.9, 29.2, 26.8, 25.2, 19.5;

HRMS (EI) m/z 430.2480 (430.2481 calcd for C₂₅H₃₀N₆O);

Anal calcd for C₂₅H₃₀N₆O.0.31H₂O: C, 68.85; H, 7.08; N, 19.27; H₂O, 1.28. Found: C, 68.62; H, 6.98; N, 19.32; H₂O, 1.68.

### Example 119

### N-{4-[4-Amino-1-(2-methylpropyl)-1H-imidazo[4,5-c]quinolin-2-yl]butyl}morpholine-4-carboxamide

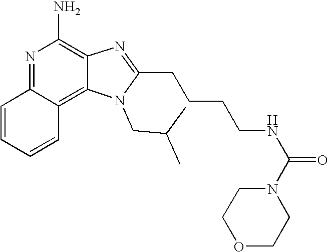

A solution of 2-(4-aminobutyl)-1-(2-methylpropyl)-1H-imidazo[4,5-c]quinolin-4-amine (0.500 g, 1.61 mmol) in dichloromethane (40 mL) was cooled to 0° C. under a nitrogen atmosphere. Triethylamine (0.250 mL, 1.76 mmol) and morpholinecarbonyl chloride (0.240 mL, 2.06 mmol) were sequentially added, and the reaction was allowed to warm to room temperature and stirred for 20 hours. An analysis by TLC indicated the presence of starting material. The solution was cooled again to 0° C., and additional morpholinecarbonyl chloride (0.050 mL) was added. The reaction was allowed to warm to room temperature; stirred for three more hours; washed sequentially with deionized water (50 mL), dilute ammonium hydroxide (50 mL), and deionized water (50 mL); dried over sodium sulfate; filtered; and concentrated under reduced pressure. The crude product was purified by column chromatography on silica gel (40 g, eluting with 90:10 dichloromethane:methanol) followed by recrystallization from ethyl acetate and hexanes. The crystals were washed with hexanes, dried in a vacuum oven, dissolved in dichloromethane, which was removed under reduced pressure, and dried under high vacuum for three days to provide 0.133 g of N-{4-[4-amino-1-(2-methylpropyl)-1H-imidazo[4,5-c]quinolin-2-yl]butyl}morpholine-4-carboxamide as a light yellow, crystalline solid, mp 97-100° C.

¹H NMR (300 MHz, DMSO-d₆) δ 7.98 (d, J=7.3 Hz, 1H), 7.62 (dd, J=8.3, 1.0 Hz, 1H), 7.419 (m, 1H), 7.27 (dt, J=8.3, 1.5 Hz, 1H), 6.56 (m, 3H), 4.34 (d, J=7.3 Hz, 2H), 3.52 (m, 4H), 3.24 (t, J=4.9 Hz, 4H), 3.11 (m, 2H), 2.92 (m, 2H), 2.17 (m, 1H), 1.81 (m, 2H), 1.56 (m, 2H), 0.93 (d, J=6.4 Hz, 6H);

¹³C NMR (125 MHz, DMSO-d₆) 157.6, 153.6, 151.5, 144.2, 132.3, 126.4, 126.2, 125.9, 121.1, 120.1, 114.7, 65.9, 51.3, 43.8, 29.4, 28.8, 26.4, 24.9, 19.1;

MS (APCI) m/z 425 (M+H);

Anal calcd for C₂₃H₃₂N₆O₂.0.18H₂O: C, 64.58; H, 7.62; N, 19.65; H₂O, 0.76. Found: C, 64.28; H, 7.74; N, 19.62; H₂O, 0.75.

### Example 120

### N-[4-(4-Amino-1-butyl-1H-imidazo[4,5-c]quinolin-2-yl)butyl]-N′-phenylurea

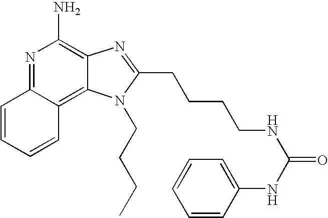

### Part A

The methods of Parts A through C of Example 116 were followed using 3-amino-4-(n-butylamino)quinoline (6.50 g, 30.2 mmol, U.S. Pat. No. 4,689,338 Example 29) as the starting material. The following modifications were made. Part A was driven to completion by heating at reflux for three days and adding a small amount of DMAP. Chromatographic purification was not carried out. In Parts B and C, the reactions did not require the addition of more reagent to drive the reaction to completion. Following Part C, 2.10 g of tert-butyl 4-(4-amino-1-butyl-1H-imidazo[4,5-c]quinolin-2-yl)butylcarbamate were obtained as a tan solid.

### Part B

Hydrogen chloride (15 mL of a 6 M solution in ethanol) was added to a solution of tert-butyl 4-(4-amino-1-butyl-1H-imidazo[4,5-c]quinolin-2-yl)butylcarbamate (2.10 g, 5.10 mmol) in ethanol (35 mL), and the reaction was heated at reflux for one hour and allowed to cool to room temperature. Nitrogen gas was bubbled through the solution, and a precipitate formed. The solvent was removed under reduced pressure, and the residue was dissolved in deionized water and adjusted to pH 11 with the addition of ammonium hydroxide. The basic mixture was extracted with chloroform (2×100 mL), and the combined extracts were dried over magnesium sulfate, filtered, and concentrated under reduced pressure to provide 1.50 g of 2-(4-aminobutyl)-1-butyl-1H-imidazo[4,5-c]quinolin-4-amine.

### Part C

The method of Part B of Example 118 was used to treat 2-(4-aminobutyl)-1-butyl-1H-imidazo[4,5-c]quinolin-4-amine (1.50 g, 4.82 mmol) with phenyl isocyanate (0.530 mL, 4.81 mmol) in dichloromethane (100 mL). After the precipitate (1.39 g) was isolated by filtration, it was recrystallized from 1,2-dichloroethane (150 mL) and a small amount of methanol. The solid was dried for two days in a vacuum oven at 80° C. to provide 0.62 g of N-[4-(4-amino-1-butyl-1H-imidazo[4,5-c]quinolin-2-yl)butyl]-N-phenylurea as a white powder, mp 222-225° C.

¹H NMR (300 MHz, DMSO-d₆) δ 8.41 (s, 1H), 8.01 (d, J=7.3 Hz, 1H), 7.61 (dd, J=8.3, 1.0 Hz, 1H), 7.43-7.37 (m, 3H), 7.28-7.18 (m, 3H), 6.87 (m 1H), 6.47 (s, 2H), 6.17 (m, 1H), 4.50 (t, J=7.3 Hz, 2H), 3.17 (m, 2H), 2.96 (m, 2H), 1.88-1.76 (m, 4H), 1.61 (m, 2H), 1.46 (m, 2H), 0.92 (t, J=7.3 Hz, 3H);

¹³C NMR (75 MHz, DMSO-d₆) 153.3, 150.9, 149.7, 142.7, 138.6, 130.3, 126.6, 124.4, 124.3, 119.2, 118.9, 118.0, 115.6, 112.8, 42.7, 29.9, 27.6, 24.2, 22.9, 17.3, 11.7;

MS (APCI) m/z 431 (M+H);

Anal calcd for C₂₅H₃₀N₆O: C, 69.74; H, 7.02; N, 19.52. Found: C, 69.50; H, 7.08; N, 19.37.

### Example 121

### N-({4-Amino-1-[2-(1,1-dioxidoisothiazolidin-2-yl)ethyl]-1H-imidazo[4,5-c]quinolin-2-yl}methyl)-N′-methylurea

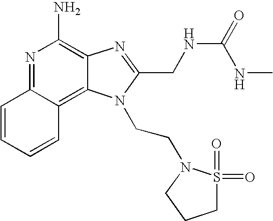

### Part A

The methods described in Parts A through E of Examples 66-115 were followed to provide tert-butyl 2-[4-amino-2-(aminomethyl)-1H-imidazo[4,5-c]quinolin-1-yl]ethylcarbamate, which was purified by column chromatography (silica gel, eluting with chloroform:2 N ammonia in methanol in a 42-minute gradient from 100:0 to 90:10) and recrystallized from isopropanol. The mother liquor from the recrystallization was concentrated under reduced pressure to provide 0.620 g (1.74 mmol) of material, which was dissolved in DMF. Methyl isocyanate (0.100 mL, 1.74 mmol) was added, and the reaction was stirred overnight at room temperature. The solvent was removed under reduced pressure, and the residue was dissolved in methanol (5 mL) and treated with hydrogen chloride (5 mL of a 4 N solution in 1,4-dioxane). The mixture was stirred overnight at room temperature. A precipitate formed and was isolated by filtration, washed with dichloromethane and diethyl ether, and dried under vacuum to provide N-{[4-amino-1-(2-aminoethyl)-1H-imidazo[4,5-c]quinolin-2-yl]methyl}-NV-methylurea hydrochloride as a white solid.

### Part B

3-Chloropropanesulfonyl chloride (0.233 mL, 1.91 mmol) and 1,8-diazabicyclo[5.4.0]undec-7-ene (DBU) (0.521 mL, 3.38 mmol) were sequentially added to a solution of the material from Part A in DMF (5 mL), and the reaction was stirred overnight at room temperature. An analysis by LC/MS indicated the reaction was incomplete, and additional DBU (0.521 mL, 3.38 mmol) was added. The reaction was stirred overnight at room temperature, and then the solvent was removed under reduced pressure. The residue was purified by normal phase prep HPLC (silica cartridge, eluting with a gradient of dichloromethane:5% ammonium hydroxide in methanol) followed by recrystallization from 1:1 acetonitrile:isopropanol. The crystals were dried in an oven for two days at 65° C. to provide 151 mg of N-({4-amino-1-[2-(1,1-dioxidoisothiazolidin-2-yl)ethyl]-1H-imidazo[4,5-c]quinolin-2-yl}methyl)-IV-methylurea, mp 229-230° C.

Anal. Calcd for C₁₈H₂₃N₇O₃S: C, 51.46; H, 5.71; N, 22.96. Found: C, 51.47; H, 5.35; N, 22.63.

### Examples 122-138

### Part A

A mixture of [4-amino-1-(2-methylpropyl)-1H-imidazo[4,5-c]quinolin-2-yl]methanol (10.9 g, 40.3 mmol, U.S. Pat. No. 5,389,640 Example 9), platinum(IV) oxide (5.5 g), and trifluoroacetic acid (75 mL) was placed under hydrogen pressure (50 psi, 3.4×10⁵ Pa) on a Parr apparatus for two days. The mixture was diluted with dichloromethane (200 mL) and filtered through CELITE filter agent; the filter cake was washed with dichloromethane. The filtrate was concentrated under reduced pressure, and the residue was partitioned between dichloromethane (200 mL) and water (200 mL). The mixture was adjusted to pH 10 with the addition of solid sodium carbonate. The aqueous layer was separated and extracted with dichloromethane (2×200 mL). A solid was present in the aqueous layer and was isolated by filtration, washed with water, and combined with the organic fractions. The combined organic fractions were concentrated under reduced pressure and purified by prep HPLC using a HORIZON HPFC system (silica gel, eluting with dichloromethane:1 M ammonia in methanol in a gradient from 95:5 to 80:20) to afford 4.92 g of [4-amino-1-(2-methylpropyl)-6,7,8,9-tetrahydro-1H-imidazo[4,5-c]quinolin-2-yl]methanol as a grey solid.

### Part B

Thionyl chloride (1.56 mL, 21.4 mmol) was added to a stirred suspension of [4-amino-1-(2-methylpropyl)-6,7,8,9-tetrahydro-1H-imidazo[4,5-c]quinolin-2-yl]methanol (4.92 g, 17.9 mmol) in 1,2-dichloroethane (180 mL). The reaction became homogeneous, and then a precipitate formed after five minutes. The reaction mixture was stirred at room temperature for 1.5 hours and concentrated under reduced pressure to yield 2-(chloromethyl)-1-(2-methylpropyl)-6,7,8,9-tetrahydro-1H-imidazo[4,5-c]quinolin-4-amine hydrochloride as a tan solid.

### Part C

A mixture of the material from Part B, potassium phthalimide (2.53 g, 13.7 mmol), potassium carbonate (4.72 g, 34.2 mmol), and DMF (75 mL) was stirred at room temperature overnight. Water (300 mL) was added. A solid was present and was isolated by filtration and washed with water and diethyl ether to provide 3.1 g of 2-{[4-amino-1-(2-methylpropyl)-6,7,8,9-tetrahydro-1H-imidazo[4,5-c]quinolin-2-yl]methyl)}-1H-isoindole-1,3(2H)-dione as a yellow solid.

### Part D

Hydrazine (0.745 mL, 15.4 mmol) was added to a stirred suspension of 2-{[4-amino-1-(2-methylpropyl)-6,7,8,9-tetrahydro-1H-imidazo[4,5-c]quinolin-2-yl]methyl)}-1H-isoindole-1,3(2H)-dione (3.1 g, 7.7 mmol) in ethanol (35 mL). After 2.5 hours at room temperature, the reaction became homogeneous. The reaction was stirred at room temperature overnight, concentrated under reduced pressure, dissolved in methanol, and purified by prep HPLC using a HORIZON HPFC system (FLASH 40+M cartridge, eluting sequentially with 90:10 chloroform:methanol and dichloromethane: 1 M ammonia in methanol in a gradient from 90:10 to 80:20) to provide 1.77 g of 2-aminomethyl-1-(2-methylpropyl)-6,7,8,9-tetrahydro-1H-imidazo[4,5-c]quinolin-4-amine as a yellow solid.

### Part E

A reagent (0.11 mmol, 1.1 equivalents) from the table below was added to a test tube containing 2-aminomethyl-1-(2-methylpropyl)-6,7,8,9-tetrahydro-1H-imidazo[4,5-c]quinolin-4-amine (27 mg, 0.10 mmol) and N,N-diisopropylethylamine (0.034 mL, 0.20 mmol) in N,N-dimethylacetamide (DMA) (1 mL). The test tube was capped and vortexed overnight at ambient temperature. Two drops of water were added to each test tube, and the solvent was removed by vacuum centrifugation.

The compounds were purified by reversed phase prep HPLC using a Waters FractionLynx automated purification system. The prep HPLC fractions were analyzed using a Waters LC/TOF-MS, and the appropriate fractions were centrifuge evaporated to provide the trifluoroacetate salt of the desired compound. Reversed phase preparative liquid chromatography was performed with non-linear gradient elution from 5-95% B where A is 0.05% trifluoroacetic acid/water and B is 0.05% trifluoroacetic acid/acetonitrile. Fractions were collected by mass-selective triggering. The table below shows the reagent added to each test tube, the structure of the resulting compound, and the observed accurate mass for the isolated trifluoroacetate salt.

### Examples 122-138

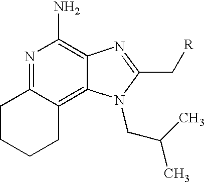

| | | | Measured Mass |
|---|---|---|---|
| Example | Reagent | R | (M + H) |
| 122 | Methyl chloroformate | 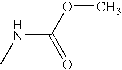 | 332.2057 |
| 123 | Ethyl chloroformate | 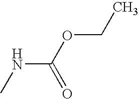 | 346.2272 |
| 124 | Ethyl isocyanate | 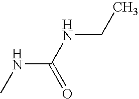 | 345.2404 |
| 125 | Methyl isothiocyanate | 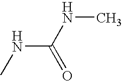 | 347.2002 |
| 126 | Phenyl isocyanate | 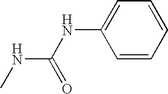 | 393.2372 |
| 127 | Phenyl isothiocyanate | 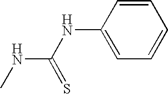 | 409.2180 |
| 128 | 3-Pyridyl isothiocyanate | 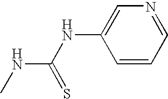 | 410.2107 |
| 129 | (R)-(+)-alpha- Methylbenzyl isocyanate | 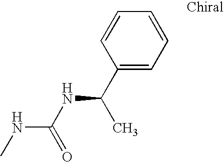 | 421.2697 |
| 130 | (S)-(−)-alpha- Methylbenzyl isocyanate | 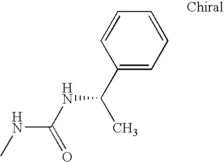 | 421.2711 |
| 131 | 3-Chlorophenyl isocyanate | 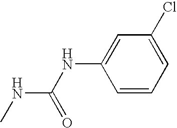 | 427.1992 |
| 132 | 4-Chlorophenyl isocyanate | 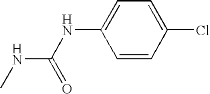 | 427.2014 |
| 133 | 3,4-Difluorophenyl isocyanate | 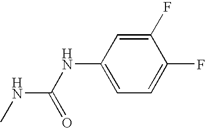 | 429.2206 |
| 134 | N,N- Dimethylcarbamoyl chloride | 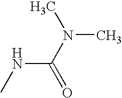 | 345.2388 |
| 135 | 1-Pyrrolidinecarbonyl chloride | 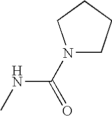 | 371.2531 |
| 136 | 1-Piperidinecarbonyl chloride | 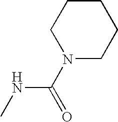 | 385.2703 |
| 137 | 4- Morpholinylcarbonyl chloride | 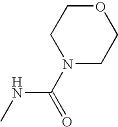 | 387.2485 |
| 138 | 4-Methyl-1- piperazinecarbonyl chloride | 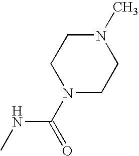 | 400.2802 |

### Example 139-161

### Part A

A solution of tert-butyl 4-[(3-amino[1,5]naphthyridin-4-yl)amino]butylcarbamate (15.3 g, 46.2 mmol, U.S. Pat. No. 6,514,985 Example 42) in dichloromethane was cooled to 0° C., and triethylamine (11.2 mL, 80.9 mmol) was added. A solution of chloroacetyl chloride (4.0 mL, 51 mmol) in dichloromethane was added dropwise. The total amount of dichloromethane used was 150 mL. The solution was allowed to warm to room temperature and stirred overnight. An analysis by LC/MS indicated the presence of starting material, and 1,2-dichloroethane was added. The reaction was heated at reflux overnight. Water was added, and the organic layer was separated and washed twice with brine. The aqueous layer was extracted with dichloromethane. The combined organic fractions were concentrated under reduce pressure and purified by normal phase prep HPLC (silica cartridge, eluting with dichloromethane:methanol:ammonium hydroxide in a gradient from 100:0:0 to 90:9.5:0.5) to provide 9.1 g of tert-butyl 4-[2-(chloromethyl)-1H-imidazo[4,5-c][1,5]naphthyridin-1-yl]butylcarbamate.

### Part B

Triethylamine (4.9 mL, 35 mmol) and potassium phthalimide (5.2 g, 28 mmol) were sequentially added to a solution of tert-butyl 4-[2-(chloromethyl)-1H-imidazo[4,5-c][1,5]naphthyridin-1-yl]butylcarbamate (9.1 g, 23 mmol) in DMF (100 mL), and the reaction was stirred overnight at room temperature, concentrated under reduced pressure, and diluted with dichloromethane. The resulting solution was washed with brine and concentrated under reduced pressure. An analysis by LC/MS indicated the presence of starting material. The product mixture was again subjected to the reaction conditions and stirred overnight at room temperature. The reaction was still incomplete, and it was heated at reflux overnight. The DMF was removed under reduced pressure, and the residue was dissolved in dichloromethane. The resulting solution was washed with brine and concentrated under reduced pressure. The crude product was purified by normal phase prep HPLC (silica cartridge, eluting with dichloromethane:methanol:ammonium hydroxide in a gradient from 100:0:0 to 95:4.7:0.3) to provide 8.0 g of tert-butyl 4-{2-[(1,3-dioxo-1,3-dihydro-2H-isoindol-2-yl)methyl]-1H-imidazo[4,5-c][1,5]naphthyridin-1-yl}butylcarbamate.

### Part C

Hydrazine (0.99 mL, 32 mmol) was added to a stirred suspension of tert-butyl 4-{2-[(1,3-dioxo-1,3-dihydro-2H-isoindol-2-yl)methyl]-1H-imidazo[4,5-c][1,5]naphthyridin-1-yl}butylcarbamate (8.0 g, 16 mmol) in ethanol (100 mL), and the reaction was stirred at room temperature overnight. An analysis by LC/MS indicated the presence of starting material, and additional hydrazine (0.99 mL) was added. The reaction was stirred at room temperature for six hours. Dichloromethane was added and removed under reduced pressure; this was repeated two additional times. The residue was purified by normal phase prep HPLC (silica cartridge, eluting with dichloromethane:methanol containing a small amount of ammonium hydroxide in a gradient from 100:0 to 90:10) to provide 3.0 g of tert-butyl 4-[2-(aminomethyl)-1H-imidazo[4,5-c][1,5]naphthyridin-1-yl]butylcarbamate as a sticky, yellow solid.

### Part D

Methyl isocyanate (607 mg, 12.2 mmol) was added dropwise to a solution of tert-butyl 4-[2-(aminomethyl)-1H-imidazo[4,5-c][1,5]naphthyridin-1-yl]butylcarbamate (3.0 g, 8.1 mmol) in pyridine (50 mL). The reaction was stirred for one hour at room temperature and concentrated under reduced pressure. The residue was dissolved in ethyl acetate, and the resulting solution was washed twice with brine and concentrated under reduced pressure to provide tert-butyl 4-[2-({[(methylamino)carbonyl]amino}methyl)-1H-imidazo[4,5-c][1,5]naphthyridin-1-yl]butylcarbamate as a brown solid.

### Part E

3-Chloroperoxybenzoic acid (3.7 g of 77% pure material, 16 mmol) was added in one portion to a solution of tert-butyl 4-[2-(f{[(methylamino)carbonyl]amino}methyl)-1H-imidazo[4,5-c][1,5]naphthyridin-1-yl]butylcarbamate (3.5 g, 8.2 mmol) in 1,2-dichloroethane (100 mL); the reaction mixture was stirred at ambient temperature for two hours. Concentrated ammonium hydroxide (100 mL) was added, and then p-toluenesulfonyl chloride (1.37 g, 9.00 mmol) was added in one portion. The reaction mixture was stirred at room temperature for two hours, and then dichloromethane and brine were added. The organic fraction was separated and was twice with brine, dried over magnesium sulfate, filtered through a layer of CELITE filter agent, and concentrated under reduced pressure to provide 3.6 g of tert-butyl 4-[4-amino-2-({[(methylamino)carbonyl]amino}methyl)-1H-imidazo[4,5-c][1,5]naphthyridin-1-yl]butylcarbamate as a brown solid.

### Part F

Hydrogen chloride (50.8 mL of a 4 M solution in 1,4-dioxane) was added to a solution of tert-butyl 4-[4-amino-2-({[(methylamino)carbonyl]amino}methyl)-1H-imidazo[4,5-c][1,5]naphthyridin-1-yl]butylcarbamate (3.0 g, 6.8 mmol) in dichloromethane. The reaction was stirred overnight at room temperature and then concentrated under reduced pressure. The residue was purified by normal phase prep HPLC (silica cartridge, eluting with dichloromethane:methanol:ammonium hydroxide in a gradient from 100:0:0 to 75:23.7:1.3) to provide 1.9 g of N-{[4-amino-1-(4-aminobutyl)-1H-imidazo[4,5-c][1,5]naphthyridin-2-yl]methyl}-N′-methylurea as an orange solid.

### Part G

A reagent (0.11 mmol, 1.1 equivalents) from the table below was added to a test tube containing N-{[4-amino-1-(4-aminobutyl)-1H-imidazo[4,5-c][1,5]naphthyridin-2-yl]methyl}-N′-methylurea (34 mg, 0.10 mmol) and N,N-diisopropylethylamine (0.034 mL, 0.20 mmol) in DMA (1 mL). The test tube was capped and vortexed overnight at ambient temperature. Water (0.100 mL) was added to each test tube, and the solvent was removed by vacuum centrifugation.

The compounds were purified by prep HPLC according to the method described in Examples 122-138. The table below shows the reagent added to each test tube, the structure of the resulting compound, and the observed accurate mass for the isolated trifluoroacetate salt.

### Examples 139-161

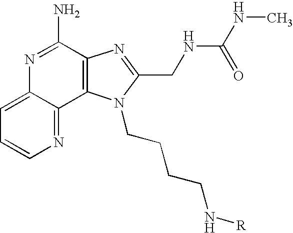

| | | | Measured Mass |
|---|---|---|---|
| Example | Reagent | R | (M + H) |
| 139 | None | 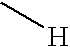 | 343.1991 |
| 140 | Propionyl chloride | 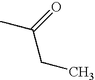 | 399.2258 |
| 141 | Cyclobutanecarbonyl chloride | 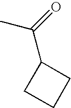 | 425.2420 |
| 142 | Cyclohexanecarbonyl chloride | 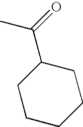 | 453.2683 |
| 143 | Hydrocinnamoyl chloride | 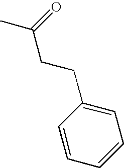 | 475.2560 |
| 144 | 3-Methoxybenzoyl chloride | 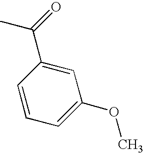 | 477.2359 |
| 145 | p-Anisoyl chloride | 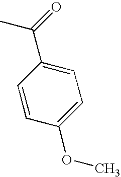 | 477.2335 |
| 146 | 3-Chorobenzoyl chloride | 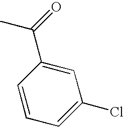 | 481.1890 |
| 147 | Nicotinoyl chloride hydrochloride | 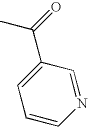 | 448.2222 |
| 148 | trans-2-Phenyl-1- Cyclopropanecarbonyl chloride | 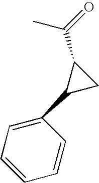 | 487.2566 |
| 149 | 1-Butanesulfonyl chloride | 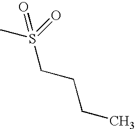 | 463.2256 |
| 150 | Benzenesulfonyl chloride | 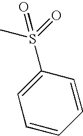 | 483.1953 |
| 151 | 1-Methylimidazole-4- sulfonyl chloride | 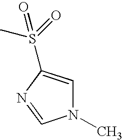 | 487.2029 |
| 152 | 4-Methoxybenzenesulfonyl chloride | 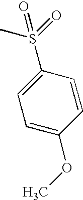 | 513.2026 |
| 153 | 3-Chlorobenzenesulfonyl chloride | 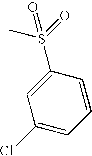 | 517.1539 |
| 154 | 4-Chlorobenzenesulfonyl chloride | 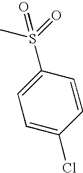 | 517.1551 |
| 155 | Ethyl isocyanate | 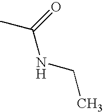 | 414.2363 |
| 156 | Phenyl isocyanate | 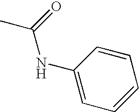 | 462.2347 |
| 157 | Cyclohexyl isocyanate | 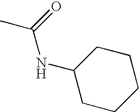 | 468.2851 |
| 158 | 3-Pyridyl isothiocyanate | 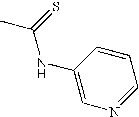 | 479.2106 |
| 159 | Benzoyl isocyanate | 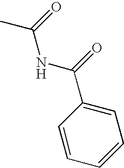 | 490.2351 |
| 160 | 4-Methoxyphenyl isocyanate | 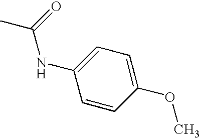 | 492.2483 |
| 161 | 4-Methyl-1- piperazinecarbonyl chloride | 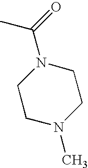 | 469.2811 |

### Examples 162-178

### Part A

A solution of N-(4-{[tert-butyl(dimethyl)silyl]oxy}butyl)quinoline-3,4-diamine (33 g, 96 mmol, U.S. Pat. No. 6,664,264 Example 1, Parts A through C) in dichloromethane (250 mL) was cooled to 0° C., and a solution of chloroacetyl chloride (8.4 mL, 105 mmol) in dichloromethane (100 mL) was added dropwise. The reaction was stirred for 30 minutes. Additional dichloromethane and then triethylamine (23.5 mL, 167 mmol) were added. The resulting solution was allowed to warm to room temperature and stirred overnight. Water was added, and the organic layer was separated and washed twice with water, dried over magnesium sulfate, filtered through a layer of CELITE filter agent, and concentrated under reduced pressure to provide 37 g of 1-(4-{[tertbutyl(dimethyl)silyl]oxy}butyl)-2-(chloromethyl)-1H-imidazo[4,5-c]quinoline as a brown oil.

### Part B

Triethylamine (15.3 mL, 109 mmol) and potassium phthalimide (20.1 g, 109 mmol) were sequentially added to a solution of 1-(4-{[tertbutyl(dimethyl)silyl]oxy}butyl)-2-(chloromethyl)-1H-imidazo[4,5-c]quinoline (37 g, 92 mmol) in DMF (240 mL), and the reaction was stirred for one hour at room temperature and quenched with water. A precipitate formed and was isolated by filtration and dissolved in dichloromethane. The resulting solution was washed with water, using ethyl acetate and brine to break up an emulsion, dried over sodium sulfate, filtered through a layer of CELITE filter agent, and concentrated under reduced pressure to provide 2-{[1-(4-{[tert-butyl(dimethyl)silyl]oxy}butyl)-1H-imidazo[4,5-c]quinolin-2-yl]methyl}-1H-isoindole-1,3(2H)-dione as a red semi-solid.

### Part C

Hydrazine (1.93 mL, 62.2 mmol) was added to a stirred suspension of 2-{[1-(4-{[tert-butyl(dimethyl)silyl]oxy}butyl)-1H-imidazo[4,5-c]quinolin-2-yl]methyl}-1H-isoindole-1,3(2H)-dione (16 g, 31 mmol) in ethanol (100 mL), and the reaction was stirred at room temperature overnight. The solvent was removed under reduced pressure. Dichloromethane was added to the residue and removed under reduced pressure; this was repeated two additional times. The residue was purified by normal phase prep HPLC (silica cartridge, eluting with dichloromethane:methanol:ammonium hydroxide in a gradient from 100:0:0 to 93:6.6:0.4) to provide 3.0 g of 2-(aminomethyl)-1-(4-{[tertbutyl(dimethyl)silyl]oxy}butyl)-1H-imidazo[4,5-c]quinolin-4-amine as a sticky, tan solid.

### Part D

Methyl isocyanate (585 mg, 11.7 mmol) was added dropwise to a solution of 2-(aminomethyl)-1-(4-{[tert-butyl(dimethyl)silyl]oxy}butyl)-1H-imidazo[4,5-c]quinolin-4-amine (3.0 g, 7.8 mmol) and triethylamine (21.7 mL, 156 mmol) in pyridine (100 mL). The reaction was stirred for two hours at room temperature and concentrated under reduced pressure. The residue was dissolved in ethyl acetate, and the resulting solution was washed twice with brine, dried over magnesium sulfate, filtered through a layer of CELITE filter agent, and concentrated under reduced pressure to provide 3 g of N-{[1-(4-{[tert-butyl(dimethyl)silyl]oxy}butyl)-1H-imidazo[4,5-c]quinolin-2-yl]methyl}-N-methylurea as a brown solid.

### Part E

The methods described in Part E of Examples 139-161 were used to oxidize and aminate N-{[1-(4-{[tert-butyl(dimethyl)silyl]oxy}butyl)-1H-imidazo[4,5-c]quinolin-2-yl]methyl}-N′-methylurea (3 g, 7 mmol) with the following modifications. The oxidation reaction was stirred for one hour, and the amination reaction was stirred overnight. At the end of the amination reaction, most of the 1,2-dichloroethane was removed under reduced pressure, and the resulting mixture was partitioned between ethyl acetate and brine. The organic layer was separated, dried, and isolated as described in Part E of Examples 139-161 to provide 3 g of N-{[4-amino-1-(4-{[tert-butyl(dimethyl)silyl]oxy}butyl)-1H-imidazo[4,5-c]quinolin-2-yl]methyl}-N′-methylurea as a brown solid.

### Part F

Water (30 mL) and concentrated acetic acid (90 mL) were sequentially added to a solution of N-{[4-amino-1-(4-{[tert-butyl(dimethyl)silyl]oxy}butyl)-1H-imidazo[4,5-c]quinolin-2-yl]methyl}-N′-methylurea (3 g, 6.5 mmol) in THF (30 mL), and the reaction was stirred at 60° C. for three days, allowed to cool to room temperature, and cooled to 0° C. Aqueous sodium hydroxide (190 mL of 6 M) was added to adjust the mixture to pH 8. The aqueous layer was washed with ethyl acetate and concentrated under reduced pressure. The residue was mixed with DMF, and a solid was removed by filtration. The filtrate was concentrated under reduced pressure and further dried under high vacuum to provide N-{[4-amino-1-(4-hydroxybutyl)-1H-imidazo[4,5-c]quinolin-2-yl]methyl}-N-methylurea acetic acid salt.

### Part G

Thionyl chloride (0.431 mL, 5.81 mmol) was added to a suspension of N-{[4-amino-1-(4-hydroxybutyl)-1H-imidazo[4,5-c]quinolin-2-yl]methyl}-N′-methylurea acetic acid salt (1.0 g, 2.9 mmol) in 1,2-dichloroethane (50 mL), and the reaction was stirred at room temperature for four hours. An analysis by LC/MS indicated the presence of starting material; additional thionyl chloride (0.215 mL, 2.89 mmol) was added. The reaction was stirred at room temperature overnight. The reaction was still incomplete, and additional thionyl chloride (0.215 mL, 2.89 mmol) was added. The reaction was stirred at room temperature for six hours and then cooled to 0° C. Methanol (10 mL) was slowly added. The volatiles were removed under reduced pressure, and the residue was dried under high vacuum to provide N-{[4-amino-1-(4-chlorobutyl)-1H-imidazo[4,5-c]quinolin-2-yl]methyl}-N′-methylurea as a yellow solid.

### Part H

A secondary amine or substituted phenol (0.15 mmol, 1.5 equivalents) from the table below was added to a test tube containing N-{[4-amino-1-(4-chlorobutyl)-1H-imidazo[4,5-c]quinolin-2-yl]methyl}-N′-methylurea (36 mg, 0.10 mmol), potassium carbonate (0.055 g, 0.40 mmol), and DMA (1 mL). For Examples 162-176, the test tube was capped, heated overnight at 70° C., and then heated at 85° C. for eight hours. For Examples 177-178, the test tube was capped, heated overnight at 85° C., and then heated at 100° C. for eight hours. Each reaction mixture was filtered, and the filter cake was washed with DMA (0.200 mL). The solvent was removed from each filtrate by vacuum centrifugation.

The compounds were purified by prep HPLC according to the method described in Examples 122-138. The table below shows the secondary amine or substituted phenol added to each test tube, the structure of the resulting compound, and the observed accurate mass for the isolated trifluoroacetate salt.

### Examples 162-178

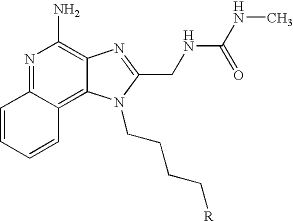

| | | | Measured Mass |
|---|---|---|---|
| Example | Reagent | R | (M + H) |
| 162 | None | 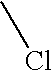 | 361.1567 |
| 163 | 3-Hydroxypiperidine | 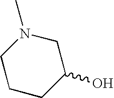 | 426.2654 |
| 164 | 4-Hydroxypiperidine | 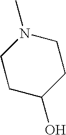 | 426.2651 |
| 165 | Thiomorpholine | 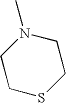 | 428.2264 |
| 166 | 3-(Dimethylamino)pyrrolidine | 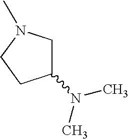 | 439.2971 |
| 167 | N,N′-Dimethyl-3- aminopyrrolidine | 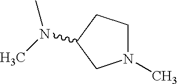 | 439.2971 |
| 168 | N-Methylhomopiperazine | 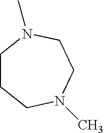 | 439.2921 |
| 169 | 3-(Hydroxymethyl)piperidine | 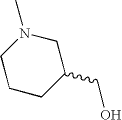 | 440.2793 |
| 170 | 4-(Hydroxymethyl)piperidine | 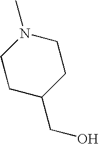 | 440.2792 |
| 171 | Isonipecotamide | 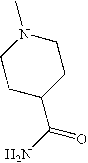 | 453.2736 |
| 172 | Nipecotamide | 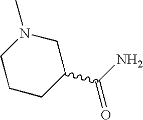 | 453.2737 |
| 173 | 1-Acetylpiperazine | 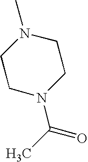 | 453.2714 |
| 174 | 1-Methyl-4- (Methylamino)piperidine | 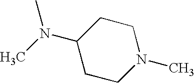 | 453.3100 |
| 175 | 4-Piperidineethanol | 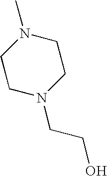 | 455.2906 |
| 176 | 4-(1-Pyrrolidinyl)- piperidine | 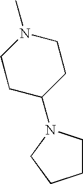 | 479.3239 |
| 177 | 3-Methoxyphenol | 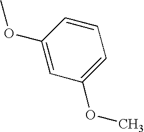 | 449.2332 |
| 178 | 3-Chlorophenol | 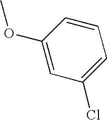 | 453.1813 |

### Examples 179-187

### Part A

A mixture of platinum(IV) oxide (0.400 g, 1.28 mmol), N-{[4-amino-1-(4-hydroxybutyl)-1H-imidazo[4,5-c]quinolin-2-yl]methyl}-N′-methylurea acetic acid salt (prepared in Part F of Examples 162-178, 0.440 mg, 1.28 mmol) and trifluoroacetic acid (50 mL) was placed under hydrogen pressure on a Parr apparatus for six days. The reaction mixture was diluted with methanol and filtered through a layer of CELITE filter agent. The filtrate was concentrated under reduced pressure. The residue was stirred with hydrogen chloride (30 mL of a 4 M solution in 1,4-dioxane), and the resulting mixture was adjusted to pH 10 with the addition of aqueous sodium hydroxide (60 mL of 4 N). The basic mixture was extracted with chloroform (3×100 mL), and the combined extracts were concentrated under reduced pressure, combined with material from another run, and purified by normal phase prep HPLC (silica cartridge, eluting with dichloromethane:methanol:ammonium hydroxide in a gradient from 100:0:0 to 90:9.5:0.5) to provide N-{[4-amino-1-(4-hydroxybutyl)-6,7,8,9-tetrahydro-1H-imidazo[4,5-c]quinolin-2-yl]methyl}-NA-methylurea as a white solid.

### Part B

Thionyl chloride (0.330 mL, 4.55 mmol) was added to a suspension of N-{[4-amino-1-(4-hydroxybutyl)-6,7,8,9-tetrahydro-1H-imidazo[4,5-c]quinolin-2-yl]methyl}-N′-methylurea (0.519 g, 1.5 mmol) in 1,2-dichloroethane (15 mL), and the reaction was stirred at room temperature overnight and then cooled to 0° C. Methanol (10 mL) was slowly added. The volatiles were removed under reduced pressure, and the residue was dried under high vacuum to provide N-{[4-amino-1-(4-chlorobutyl)-6,7,8,9-tetrahydro-1H-imidazo[4,5-c]quinolin-2-yl]methyl}-N′-methylurea as a white solid.

### Part C

A secondary amine or substituted phenol (0.15 mmol, 1.5 equivalents) from the table below was added to a test tube containing N-{[4-amino-1-(4-chlorobutyl)-6,7,8,9-tetrahydro-1H-imidazo[4,5-c]quinolin-2-yl]methyl}-N′-methylurea (36 mg, 0.10 mmol), potassium carbonate (0.055 g, 0.40 mmol), and DMA (1 mL). The test tube was capped and heated overnight at 70° C. Each reaction mixture was allowed to cool to room temperature and filtered, and the filter cake was washed with DMA (0.200 mL). The solvent was removed from each filtrate by vacuum centrifugation.

The compounds were purified by prep HPLC according to the method described in Examples 122-138. The table below shows the secondary amine or substituted phenol added to each test tube, the structure of the resulting compound, and the observed accurate mass for the isolated trifluoroacetate salt.

### Examples 179-187

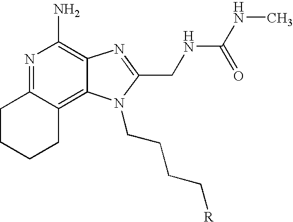

| | | | Measured Mass |
|---|---|---|---|
| Example | Reagent | R | (M + H) |
| 179 | None | 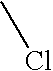 | 365.1867 |
| 180 | Piperidine | 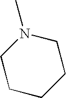 | 414.2967 |
| 181 | Morplioline | 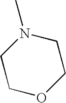 | 416.2776 |
| 182 | 3-(Dimethylamino)pyrrolidine | 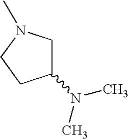 | 443.3268 |
| 183 | N,N′-Dimethyl-3- aminopyrrolidine | 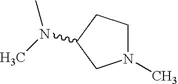 | 443.3273 |
| 184 | N-Methylhomopiperazine | 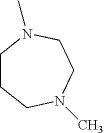 | 443.3257 |
| 185 | N-Methylbenzylamine | 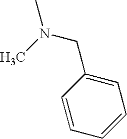 | 450.2996 |
| 186 | 1 -Acetylpiperazine | 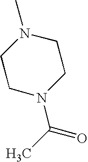 | 457.3043 |
| 187 | 4-Piperidineethanol | 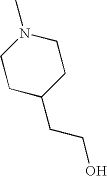 | 458.3247 |

### Example 188

### N′-({4-Amino-1-[(1-hydroxycyclohexyl)methyl]-1H-imidazo[4,5-c]quinolin-2-yl}methyl)-N,N-dimethylurea

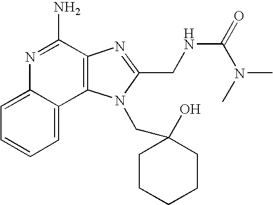

### Part A

A suspension of 1-aminomethyl-1-cyclohexanol hydrochloride (20.0 g, 121 mmol) and 4-chloro-3-nitroquinoline (24.0 g, 115 mmol) in dichloromethane (550 mL) was cooled to 0° C., and triethylamine (40 mL, 290 mmol) was added dropwise over a period of 30 minutes. The reaction was allowed to warm to room temperature over two hours. An analysis by HPLC indicated that the 4-chloro-3-nitroquinoline starting material actually contained some 3-nitroquinolin-4-ol, and additional pure 4-chloro-3-nitroquinoline (2.0 g, 57.5 mmol) was added. The reaction was stirred for four hours, and additional 1-aminomethyl-1-cyclohexanol hydrochloride (2.0 g, 12 mmol) was added, and the resulting suspension was stirred for three days. The solvent was removed under reduced pressure, and the residue was triturated in water for one hour and isolated by filtration. The resulting solid was triturated with hot dichloromethane and isolated by filtration from the hot mixture to provide 36.5 g of 1-{[(3-nitroquinolin-4-yl)amino]methyl}cyclohexanol as a bright yellow powder.

### Part B

A suspension of 1-{[(3-nitroquinolin-4-yl)amino]methyl}cyclohexanol (15.0 g, 49.8 mmol) in ethyl acetate (225 mL) in a Parr vessel was purged with nitrogen; 5% platinum on carbon (1.5 g) was added. The reaction was placed under hydrogen pressure (35 psi, 2.4×10⁵ Pa) for 3.5 hours and then filtered through a layer of CELITE filter agent. The filter cake was washed with ethyl acetate (100 mL), and the filtrate was concentrated under reduced pressure to provide 1-{[(3-aminoquinolin-4-yl)amino]methyl}cyclohexanol as a yellow solid.

### Part C

A solution of the material from Part B in dichloromethane (200 mL) was cooled to 0° C., and chloroacetyl chloride (4.4 mL, 55 mmol) was added over a period of ten minutes. The reaction was stirred for one hour at 0° C. and then concentrated under reduced pressure to provide 2-chloro-N-(4-{[(1-hydroxycyclohexyl)methyl]amino}quinolin-3-yl)acetamide hydrochloride as a yellow solid.

### Part D

Triethylamine (21 mL, 150 mmol) was added to a solution of the material from Part C in ethanol (200 mL), and the reaction was heated at 60° C. for four hours. The solvent was removed under reduced pressure, and the residue was partitioned between dichloromethane (150 mL) and saturated aqueous sodium bicarbonate (125 mL). The aqueous layer was separated and extracted with dichloromethane (2×50 mL), and the combined organic fractions were dried over magnesium sulfate, filtered, and concentrated under reduced pressure to provide 14.2 g of an orange solid. The solid was triturated with acetonitrile and isolated by filtration to provide 10.74 g of 1-{[2-(chloromethyl)-1H-imidazo[4,5-c]quinolin-1-yl]methyl}cyclohexanol as a pale yellow solid.

### Part E

3-Chloroperoxybenzoic acid (8.37 g of 70% pure material, 34 mmol) was added to a suspension of 1-{[2-(chloromethyl)-1H-imidazo[4,5-c]quinolin-1-yl]methyl}cyclohexanol (8.0 g, 24 mmol) in chloroform (100 mL), and the reaction was stirred at room temperature for four hours. Saturated aqueous sodium bicarbonate (100 mL) was added, and the mixture was stirred for 15 minutes. A precipitate formed and was isolated by filtration to provide 1-{[2-(chloromethyl)-5-oxido-1H-imidazo[4,5-c]quinolin-1-yl]methyl}cyclohexanol as a white solid.

### Part F

Ammonium hydroxide (8 mL of 15 M) was added to a suspension of the material from Part E in methanol (100 mL). The mixture was cooled to 0° C., and benzenesulfonyl chloride (6.5 mL, 51 mmol) was added dropwise over a period of eight minutes. The reaction was stirred at 0° C. for one hour, and an analysis by HPLC indicated the presence of starting material. Additional benzenesulfonyl chloride (6.5 mL, 51 mmol) was added in two portions over two hours. The reaction was allowed to warm to room temperature slowly and stirred overnight. A precipitate was present and was isolated by filtration, stirred with saturated aqueous sodium bicarbonate (100 mL), isolated by filtration, washed with water (50 mL), and dried to provide 6.14 g of 1-{[4-amino-2-(chloromethyl)-1H-imidazo[4,5-c]quinolin-1-yl]methyl}cyclohexanol.

### Part G

Potassium phthalimide (2.59 g, 14.0 mmol) was added to a solution of 1-{[4-amino-2-(chloromethyl)-1H-imidazo[4,5-c]quinolin-1-yl]methyl}cyclohexanol (5.07 g, 13.3 mmol) in DMF (50 mL), and the reaction mixture was stirred at room temperature overnight. An analysis by HPLC indicated the presence of starting material, and additional potassium phthalimide (1 g) was added. The reaction was stirred for an additional five hours, and then concentrated under reduced pressure. The residue was triturated with methanol, and the resulting white solid was isolated by filtration. The filtrate was concentrated under reduced pressure, and the residue was triturated with methanol to afford additional white solid, which was isolated by filtration. The two solids were combined to provide 2-({4-amino-1-[(1-hydroxycyclohexyl)methyl]-1H-imidazo[4,5-c]quinolin-2-yl}methyl)-1H-isoindole-1,3(2H)-dione.

### Part H

Hydrazine (2.1 mL, 66 mmol) was added to a suspension of the material from Part G in ethanol (50 mL), and the reaction was stirred for 24 hours at room temperature. The ethanol was removed under reduced pressure, and the resulting white solid was sonicated with hydrochloric acid (50 mL of 1M). The resulting suspension was filtered to remove a solid, and the filtrate was adjusted to pH 8 with the addition of solid sodium bicarbonate. A precipitate formed and was isolated by filtration and dried at 50° C. overnight in a vacuum oven to provide 2.99 g of 1-{[4-amino-2-(aminomethyl)-1H-imidazo[4,5-c]quinolin-1-yl]methyl}cyclohexanol as a white powder.

### Part I

Dimethylcarbamyl chloride (0.17 mL, 1.8 mmol) was added dropwise to a suspension of 1-{[4-amino-2-(aminomethyl)-1H-imidazo[4,5-c]quinolin-1-yl]methyl}cyclohexanol (0.595 g, 1.83 mmol) and triethylamine (0.33 mL, 2.4 mmol) in dichloromethane (20 mL), and the reaction was stirred for 24 hours at room temperature. An analysis by HPLC indicated that no reaction had taken place. 4-Dimethylaminopyridine (20 mg) was added, and the reaction was heated at reflux for seven days. Additional dimethylcarbamyl chloride (0.25 equivalent) was added on the fifth day. Dichloromethane (25 mL) and saturated aqueous sodium bicarbonate (50 mL) were added, but the layers did not separate. The mixture was concentrated under reduced pressure, and the residue was triturated with water. A white solid was present and was isolated by filtration and was purified by prep HPLC using a HORIZON HPFC system (FLASH 40+M column, eluting with chloroform:CMA in a gradient from 100:0 to 55:45) and dried in a vacuum oven for three days at 85° C. to provide 0.321 g of N-({4-amino-1-[(1-hydroxycyclohexyl)methyl]-1H-imidazo[4,5-c]quinolin-2-yl}methyl)-N,N-dimethylurea as a white powder, mp is greater than 250° C. Anal. Calcd for C₂₁H₂₈N₆O₂: C, 63.62; H, 7.12; N, 21.20. Found: C, 63.42; H, 6.93; N, 21.11.

### Examples 189-200

A reagent (0.11 mmol, 1.1 equivalents) from the table below was added to a test tube containing 1-{[4-amino-2-(aminomethyl)-1H-imidazo[4,5-c]quinolin-1-yl]methyl}cyclohexanol (33 mg, 0.10 mmol) and N,N-diisopropylethylamine (0.035 mL, 0.20 mmol) in DMA (1 mL). The test tube was capped and shaken overnight at ambient temperature. The solvent was then removed by vacuum centrifugation.

The compounds were purified by prep HPLC according to the method described in Examples 122-138. The table below shows the isocyanate or carbamoyl chloride added to each test tube, the structure of the resulting compound, and the observed accurate mass for the isolated trifluoroacetate salt.

### Examples 189-200

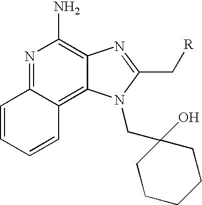

| | | | Measured Mass |
|---|---|---|---|
| Example | Reagent | R | (M + H) |
| 189 | Methyl chloroformate | 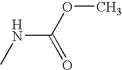 | 384.2025 |
| 190 | Methyl isocyanate | 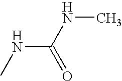 | 383.2161 |
| 191 | Ethyl isocyanate | 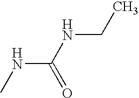 | 397.2356 |
| 192 | Isopropyl isocyanate | 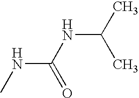 | 411.2500 |
| 193 | n-Propyl isocyanate | 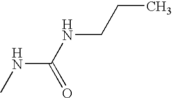 | 411.2512 |
| 194 | Phenyl isocyanate | 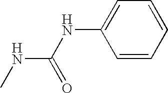 | 445.2344 |
| 195 | 3-Pyridyl isothiocyanate | 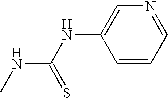 | 462.2056 |
| 196 | (R)-(+)-alpha- Methylbenzyl isocyanate | 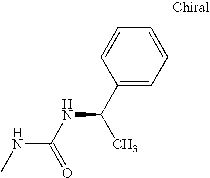 | 473.2672 |
| 197 | (S)-(−)-alpha- Methylbenzyl isocyanate | 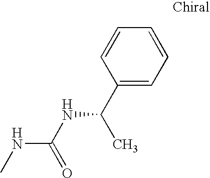 | 473.2676 |
| 198 | N,N- Dimethylcarbamoyl chloride | 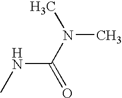 | 397.2336 |
| 199 | 1-Piperidinecarbonyl chloride | 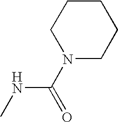 | 437.2650 |
| 200 | 4- Morpholinylcarbonyl chloride | 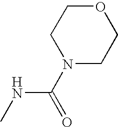 | 439.2470 |

### Examples 201-212

### Part A

Under a nitrogen atmosphere, nitromethane (116 mL, 2.14 mol) and sodium ethoxide (2.6 g of 96% pure material, 36 mmol) were sequentially added to a solution of cyclobutanone (50.0 g, 713 mmol) in ethanol (71 mL), and the resulting solution was stirred at room temperature for three days. Some of the ethanol was removed under reduced pressure, and water (100 mL) was added. The resulting mixture was extracted with ethyl acetate (3×150 mL). The combined extracts were washed sequentially with water (2×80 mL) and brine (40 mL), dried over sodium sulfate, filtered, and concentrated under reduced pressure. The residue was purified by vacuum distillation under high vacuum at 70° C. to provide 46.1 g of 1-(nitromethyl)cyclobutanol as an orange liquid.

### Part B

A mixture of 1-(nitromethyl)cyclobutanol (46.0 g, 351 mmol), 20% palladium hydroxide on carbon (6.9 g) and ethanol (1 L) was placed under hydrogen pressure (30 psi, 2.1×10⁵ Pa) on a Parr apparatus for two days. An analysis by nuclear magnetic resonance spectoscopy indicated the reaction was incomplete, and additional 20% palladium hydroxide on carbon (5 g) was added. The reaction was placed under hydrogen pressure (30 psi, 2.1×10⁵ Pa) for four days. The reaction mixture was filtered through a layer of CELITE filter agent, and the filter cake was washed with methanol. The filtrate was concentrated under reduced pressure to provide 34.8 g of 1-(aminomethyl)cyclobutanol as a white solid.

### Part C

A solution of 4-chloro-3-nitroquinoline (30.0 g, 144 mmol) in dichloromethane (350 mL) was cooled to 0° C. under a nitrogen atmosphere, and triethylamine (22.1 mL, 158 mmol) was added. A solution of 1-(aminomethyl)cyclobutanol (16.0 g, 158 mmol) in dichloromethane (130 mL) was then added over a period of one hour, followed by a rinse of dichloromethane (100 mL). The reaction was stirred at room temperature overnight. The solvent was removed under reduced pressure, and the residue was triturated in water (500 mL) and saturated aqueous sodium bicarbonate (200 mL) for two hours. A solid was present and was isolated by filtration, washed with a large amount of water, and dried in a vacuum oven at 55° C. to provide 38.7 g of 1-{[(3-nitroquinolin-4-yl)amino]methyl}cyclobutanol as a yellow solid.

### Part D

1-{[(3-Nitroquinolin-4-yl)amino]methyl}cyclobutanol (14.0 g, 51.2 mmol) was hydrogenated (50 psi, 3.5×10⁵ Pa) according to the method described in Part B of Example 188 to provide 1-{[(3-aminoquinolin-4-yl)amino]methyl}cyclobutanol as a yellow solid.

### Part E

A solution of the material from Part D in dichloromethane (250 mL) was cooled to 0° C., and chloroacetyl chloride (4.50 mL, 56.4 mmol) was added over a period of 15 minutes. The reaction was allowed to warm to room temperature and stirred for four hours. An analysis by LC/MS indicated the presence of starting material, and additional chloroacetyl chloride (1 mL) was added. The reaction was stirred overnight at room temperature and then concentrated under reduced pressure to provide 2-chloro-N-(4-{[(1-hydroxycyclobutyl)methyl]amino}quinolin-3-yl)acetamide hydrochloride.

### Part F

The method described in Part D of Example 188 was used to treat the material from Part E with triethylamine (21.4 mL, 154 mmol) with the modifications that the reaction was heated at 50° C. for four hours, chloroform was used in the work-up procedure, and following the work-up procedure, the product was not purified by trituration. 1-{[2-(Chloromethyl)-1H-imidazo[4,5-c]quinolin-1-yl]methyl}cyclobutanol (16.5 g) was obtained as a yellow solid containing small amounts of chloroform and triethylamine.

### Part G

3-Chloroperoxybenzoic acid (9.15 g of 70% pure material, 37.1 mmol) was added to a suspension of 1-{[2-(chloromethyl)-1H-imidazo[4,5-c]quinolin-1-yl]methyl}cyclobutanol (8.00 g, 26.5 mmol) in chloroform (100 mL) under a nitrogen atmosphere, and the reaction was stirred at room temperature overnight. Additional chloroform (200 mL) was added, and the solution was washed sequentially with saturated aqueous sodium bicarbonate (2×80 mL) and brine (20 mL), dried over magnesium sulfate, filtered, and concentrated under reduced pressure to provide 1-{[2-(chloromethyl)-5-oxido-1H-imidazo[4,5-c]quinolin-1-yl]methyl}cyclobutanol as a yellow solid.

### Part H

Ammonium hydroxide (8.83 mL of 15 M) was added to a solution of the material from Part G in methanol (100 mL). The mixture was cooled to 0° C. under a nitrogen atmosphere, and benzenesulfonyl chloride (7.10 mL, 55.7 mmol) was added dropwise over a period of eight minutes. The reaction was stirred at 0° C. for two hours, combined with material from another run, and concentrated under reduced pressure. The residue was dissolved in chloroform (300 mL), and the resulting solution was washed sequentially with saturated aqueous sodium carbonate (2×80 mL) and brine (40 mL), dried over sodium sulfate, filtered, and concentrated under reduced pressure. The crude product was purified by prep HPLC using a HORIZON HPFC system (silica cartridge, eluting with chloroform:CMA in a gradient from 100:0 to 70:30) to provide 4.00 g of 1-{[4-amino-2-(chloromethyl)-1H-imidazo[4,5-c]quinolin-1-yl]methyl}cyclobutanol as a yellow solid.

### Part I

Under a nitrogen atmosphere, potassium phthalimide (1.21 g, 6.52 mmol) was added to a solution of 1-{[4-amino-2-(chloromethyl)-1H-imidazo[4,5-c]quinolin-1-yl]methyl}cyclobutanol (1.88 g, 5.93 mmol) in DMF (30 mL), and the reaction mixture was stirred at room temperature overnight and then concentrated under reduced pressure. The residue was partitioned between chloroform (200 mL) and water (25 mL)/saturated aqueous sodium bicarbonate (2×40 mL), dried over sodium sulfate, filtered, and concentrated under reduced pressure to provide 2.42 g of 2-({4-amino-1-[(1-hydroxycyclobutyl)methyl]-1H-imidazo[4,5-c]quinolin-2-yl}methyl)-1H-isoindole-1,3(2H)-dione.

### Part J

Under a nitrogen atmosphere, hydrazine (0.89 mL, 28 mmol) was added to a suspension of 2-({4-amino-1-[(1-hydroxycyclobutyl)methyl]-1H-imidazo[4,5-c]quinolin-2-yl}methyl)-1H-isoindole-1,3(2H)-dione (2.42 g, 5.66 mmol) in ethanol (57 mL), and the reaction was stirred for two hours at room temperature. The ethanol was removed under reduced pressure, and the resulting white solid was triturated with 2 N hydrochloric acid. The resulting suspension was filtered to remove a solid, and the filter cake was washed with water. The filtrate was made basic with the addition of solid sodium bicarbonate. A precipitate formed and was isolated by filtration, washed with water, and dried at 50° C. for three days in a vacuum oven to provide 0.994 g of 1-{[4-amino-2-(aminomethyl)-1H-imidazo[4,5-c]quinolin-1-yl]methyl}cyclobutanol as a yellow solid.

### Part K

A reagent (0.11 mmol, 1.1 equivalents) from the table below was added to a test tube containing 1-{[4-amino-2-(aminomethyl)-1H-imidazo[4,5-c]quinolin-1-yl]methyl}cyclobutanol (30 mg, 0.10 mmol) and N,N-diisopropylethylamine (0.036 mL, 0.20 mmol) in DMA (1 mL). The test tube was capped and vortexed overnight at ambient temperature. Two drops of water were added to each reaction, and the solvent was then removed by vacuum centrifugation.

The compounds were purified by prep HPLC according to the method described in Example 122-138. The table below shows the isocyanate or carbamoyl chloride added to each test tube, the structure of the resulting compound, and the observed accurate mass for the isolated trifluoroacetate salt.

### Examples 201-212

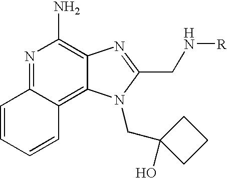

| | | | Measured |
|---|---|---|---|
| Ex- | | | Mass |
| ample | Reagent | R | (M + H) |
| | None | —H | 298.1677 |
| 201 | Methyl chloroformate | 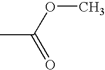 | 356.1715 |
| 202 | Methyl isocyanate | 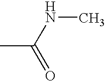 | 355.1904 |
| 203 | Ethyl isocyanate | 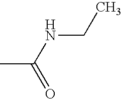 | 369.2061 |
| 204 | Isopropyl isocyanate | 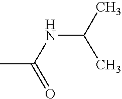 | 383.2231 |
| 205 | Cyclopropyl isothiocyanate | 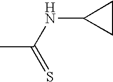 | 397.1817 |
| 206 | Cyclopropylmethyl isothiocyanate | 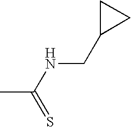 | 411.1964 |
| 207 | Phenylisocyanate | 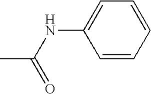 | 417.2057 |
| 208 | Phenyl isothiocyanate | 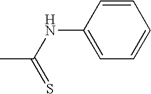 | 433.1826 |
| 209 | 3-Pyridyl isothiocyanate | 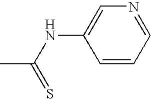 | 434.1765 |
| 210 | N,N- Dimethylcarbamoyl chloride | 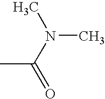 | 369.2035 |
| 211 | 1- Pyrrolidinecarbonyl chloride | 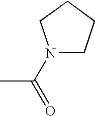 | 395.2226 |
| 212 | 4- Morpholinylcarbonyl chloride | 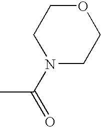 | 411.2164 |

Exemplary Compounds

Certain exemplary compounds, including some of those described above in the Examples, have the following Formulas (IIb, IIIi, IVc, or Vb) and the following R₁, and R₂₋₁ substituents, wherein each line of the table is matched with Formula IIb, IIIi, IVc, or Vb to represent a specific embodiment of the invention.

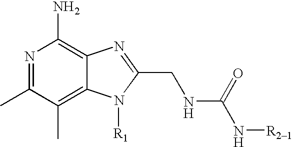

IIb

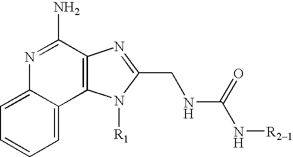

IIIi

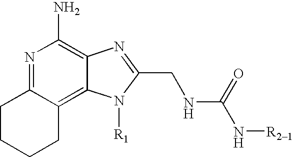

IVc

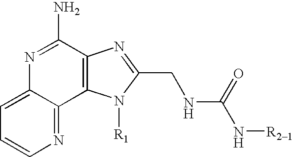

Vb

| R₁ | R₂₋₁ |
|---|---|
| 2-methylpropyl | hydrogen |
| 2-methylpropyl | methyl |
| 2-methylpropyl | ethyl |
| 2-hydroxy-2-methylpropyl | hydrogen |
| 2-hydroxy-2-methylpropyl | methyl |
| 2-hydroxy-2-methylpropyl | ethyl |
| 2-methyl-2-[(methylsulfonyl)amino]propyl | hydrogen |
| 2-methyl-2-[(methylsulfonyl)amino]propyl | methyl |
| 2-methyl-2-[(methylsulfonyl)amino]propyl | ethyl |
| 2-(cyclohexanecarbonylamino)-2-methylpropyl | hydrogen |
| 2-(cyclohexanecarbonylamino)-2-methylpropyl | methyl |
| 2-(cyclohexanecarbonylamino)-2-methylpropyl | ethyl |
| 4-[(methylsulfonyl)amino]butyl | hydrogen |
| 4-[(methylsulfonyl)amino]butyl | methyl |
| 4-[(methylsulfonyl)amino]butyl | ethyl |
| 4-(isobutyrylamino)butyl | hydrogen |
| 4-(isobutyrylamino)butyl | methyl |
| 4-(isobutyrylamino)butyl | ethyl |
| 4-[(morpholine-4-carbonyl)amino]butyl | hydrogen |
| 4-[(morpholine-4-carbonyl)amino]butyl | methyl |
| 4-[(morpholine-4-carbonyl)amino]butyl | ethyl |
| 3-methoxypropyl | hydrogen |
| 3-methoxypropyl | methyl |
| 3-methoxypropyl | ethyl |
| (1-hydroxycyclohexyl)methyl | hydrogen |
| (1-hydroxycyclohexyl)methyl | methyl |
| (1-hydroxycyclohexyl)methyl | ethyl |
| (1-hydroxycyclobutyl)methyl | hydrogen |
| (1-hydroxycyclobutyl)methyl | methyl |
| (1-hydroxycyclobutyl)methyl | ethyl |
| tetrahydro-2H-pyran-4-ylmethyl | hydrogen |
| tetrahydro-2H-pyran-4-ylmethyl | methyl |
| tetrahydro-2H-pyran-4-ylmethyl | ethyl |

### Cytokine Induction in Human Cells

Compounds of the invention have been found to modulate cytokine biosynthesis by inducing the production of interferon α and/or tumor necrosis factor α in human cells when tested using the method described below.

An in vitro human blood cell system is used to assess cytokine induction. Activity is based on the measurement of interferon (α) and tumor necrosis factor (a) (IFN-α and TNF-α, respectively) secreted into culture media as described by Testerman et. al. in “Cytokine Induction by the Immunomodulators Imiquimod and S-27609”, Journal of Leukocyte Biology, 58, 365-372 (September, 1995).

Blood Cell Preparation for Culture

Whole blood from healthy human donors is collected by venipuncture into vacutainer tubes or syringes containing EDTA. Peripheral blood mononuclear cells (PBMC) are separated from whole blood by density gradient centrifugation using HISTOPAQUE-1077 (Sigma, St. Louis, Mo.) or Ficoll-Paque Plus (Amersham Biosciences Piscataway, N.J.). Blood is diluted 1:1 with Dulbecco's Phosphate Buffered Saline (DPBS) or Hank's Balanced Salts Solution (HBSS). Alternately, whole blood is placed in Accuspin (Sigma) or LeucoSep (Greiner Bio-One, Inc., Longwood, Fla.) centrifuge frit tubes containing density gradient medium. The PBMC layer is collected and washed twice with DPBS or HBSS and re-suspended at 4×10⁶ cells/mL in RPMI complete. The PBMC suspension is added to 96 well flat bottom sterile tissue culture plates containing an equal volume of RPMI complete media containing test compound.

Compound Preparation

The compounds are solubilized in dimethyl sulfoxide (DMSO). The DMSO concentration should not exceed a final concentration of 1% for addition to the culture wells. The compounds are generally tested at concentrations ranging from 30-0.014 μM. Controls include cell samples with media only, cell samples with DMSO only (no compound), and cell samples with reference compound.

Incubation

The solution of test compound is added at 60 μM to the first well containing RPMI complete and serial 3 fold dilutions are made in the wells. The PBMC suspension is then added to the wells in an equal volume, bringing the test compound concentrations to the desired range (usually 30-0.014 μM). The final concentration of PBMC suspension is 2×10⁶ cells/mL. The plates are covered with sterile plastic lids, mixed gently and then incubated for 18 to 24 hours at 37° C. in a 5% carbon dioxide atmosphere.

Separation

Following incubation the plates are centrifuged for 10 minutes at 1000 rpm (approximately 200×g) at 4° C. The cell-free culture supernatant is removed and transferred to sterile polypropylene tubes. Samples are maintained at −30 to −70° C. until analysis. The samples are analyzed for IFN-α by ELISA and for TNF-α by IGEN/BioVeris Assay.

Interferon (α) and Tumor Necrosis Factor (α) Analysis

IFN-α concentration is determined with a human multi-subtype colorimetric sandwich ELISA (Catalog Number 41105) from PBL Biomedical Laboratories, Piscataway, N.J. Results are expressed in pg/mL.

The TNF-α concentration is determined by ORIGEN M-Series Immunoassay and read on an IGEN M-8 analyzer from BioVeris Corporation, formerly known as IGEN International, Gaithersburg, Md. The immunoassay uses a human TNF-α capture and detection antibody pair (Catalog Numbers AHC3419 and AHC3712) from Biosource International, Camarillo, Calif. Results are expressed in pg/mL.

Assay Data and Analysis

In total, the data output of the assay consists of concentration values of TNF-α and IFN-α (y-axis) as a function of compound concentration (x-axis).

Analysis of the data has two steps. First, the greater of the mean DMSO (DMSO control wells) or the experimental background (usually 20 pg/mL for IFN-α and 40 pg/mL for TNF-α) is subtracted from each reading. If any negative values result from background subtraction, the reading is reported as “*”, and is noted as not reliably detectable. In subsequent calculations and statistics, “*”, is treated as a zero. Second, all background subtracted values are multiplied by a single adjustment ratio to decrease experiment to experiment variability. The adjustment ratio is the area of the reference compound in the new experiment divided by the expected area of the reference compound based on the past 61 experiments (unadjusted readings). This results in the scaling of the reading (y-axis) for the new data without changing the shape of the dose-response curve. The reference compound used is 2-[4-amino-2-ethoxymethyl-6,7,8,9-tetrahydro-α,α-dimethyl-1H-imidazo[4,5-c]quinolin-1-yl]ethanol hydrate (U.S. Pat. No. 5,352,784; Example 91) and the expected area is the sum of the median dose values from the past 61 experiments.

The minimum effective concentration is calculated based on the background-subtracted, reference-adjusted results for a given experiment and compound. The minimum effective concentration (μmolar) is the lowest of the tested compound concentrations that induces a response over a fixed cytokine concentration for the tested cytokine (usually 20 pg/mL for IFN-α and 40 pg/mL for TNF-α). The maximal response is the maximal amount of cytokine (pg/ml) produced in the dose-response.

### Cytokine Induction in Human Cells

### High Throughput Screen

The CYTOKINE INDUCTION IN HUMAN CELLS test method described above was modified as follows for high throughput screening.

Blood Cell Preparation for Culture

Whole blood from healthy human donors is collected by venipuncture into vacutainer tubes or syringes containing EDTA. Peripheral blood mononuclear cells (PBMC) are separated from whole blood by density gradient centrifugation using HISTOPAQUE-1077 (Sigma, St. Louis, Mo.) or Ficoll-Paque Plus (Amersham Biosciences Piscataway, N.J.). Whole blood is placed in Accuspin (Sigma) or LeucoSep (Greiner Bio-One, Inc., Longwood, Fla.) centrifuge frit tubes containing density gradient medium. The PBMC layer is collected and washed twice with DPBS or HBSS and re-suspended at 4×10⁶ cells/mL in RPMI complete (2-fold the final cell density). The PBMC suspension is added to 96-well flat bottom sterile tissue culture plates.

Compound Preparation

The compounds are solubilized in dimethyl sulfoxide (DMSO). The compounds are generally tested at concentrations ranging from 30-0.014 μM. Controls include cell samples with media only, cell samples with DMSO only (no compound), and cell samples with a reference compound 2-[4-amino-2-ethoxymethyl-6,7,8,9-tetrahydro-α,α-dimethyl-1H-imidazo[4,5-c]quinolin-1-yl]ethanol hydrate (U.S. Pat. No. 5,352,784; Example 91) on each plate. The solution of test compound is added at 7.5 mM to the first well of a dosing plate and serial 3 fold dilutions are made for the 7 subsequent concentrations in DMSO. RPMI Complete media is then added to the test compound dilutions in order to reach a final compound concentration of 2-fold higher (60-0.028 LM) than the final tested concentration range.

Incubation

Compound solution is then added to the wells containing the PBMC suspension bringing the test compound concentrations to the desired range (usually 30-0.014 μM) and the DMSO concentration to 0.4%. The final concentration of PBMC suspension is 2×10⁶ cells/mL. The plates are covered with sterile plastic lids, mixed gently and then incubated for 18 to 24 hours at 37° C. in a 5% carbon dioxide atmosphere.

Separation

Following incubation the plates are centrifuged for 10 minutes at 1000 rpm (approximately 200 g) at 4° C. 4-plex Human Panel MSD MULTI-SPOT 96-well plates are pre-coated with the appropriate capture antibodies by MesoScale Discovery, Inc. (MSD, Gaithersburg, Md.). The cell-free culture supernatants are removed and transferred to the MSD plates. Fresh samples are typically tested, although they may be maintained at −30 to −70° C. until analysis.

Interferon-α and Tumor Necrosis Factor-α Analysis

MSD MULTI-SPOT plates contain within each well capture antibodies for human TNF-α and human IFN-α that have been pre-coated on specific spots. Each well contains four spots: one human TNF-α capture antibody (MSD) spot, one human IFN-α capture antibody (PBL Biomedical Laboratories, Piscataway, N.J.) spot, and two inactive bovine serum albumin spots. The human TNF-α capture and detection antibody pair is from MesoScale Discovery. The human IFN-α multi-subtype antibody (PBL Biomedical Laboratories) captures all IFN-α subtypes except IFN-α F (IFNA21). Standards consist of recombinant human TNF-α (R&D Systems, Minneapolis, Minn.) and IFN-α (PBL Biomedical Laboratories). Samples and separate standards are added at the time of analysis to each MSD plate. Two human IFN-α detection antibodies (Cat. Nos. 21112 & 21100, PBL) are used in a two to one ratio (weight:weight) to each other to determine the IFN-α concentrations. The cytokine-specific detection antibodies are labeled with the SULFO-TAG reagent (MSD). After adding the SULFO-TAG labeled detection antibodies to the wells, each well's electrochemoluminescent levels are read using MSD's SECTOR HTS READER. Results are expressed in pg/mL upon calculation with known cytokine standards.

Assay Data and Analysis

In total, the data output of the assay consists of concentration values of TNF-α or IFN-α (y-axis) as a function of compound concentration (x-axis).

A plate-wise scaling is performed within a given experiment aimed at reducing plate-to-plate variability associated within the same experiment. First, the greater of the median DMSO (DMSO control wells) or the experimental background (usually 20 pg/mL for IFN-α and 40 pg/mL for TNF-α) is subtracted from each reading. Negative values that may result from background subtraction are set to zero. Each plate within a given experiment has a reference compound that serves as a control. This control is used to calculate a median expected area under the curve across all plates in the assay. A plate-wise scaling factor is calculated for each plate as a ratio of the area of the reference compound on the particular plate to the median expected area for the entire experiment. The data from each plate are then multiplied by the plate-wise scaling factor for all plates. Only data from plates bearing a scaling factor of between 0.5 and 2.0 (for both cytokines IFN-α, TNF-α) are reported. Data from plates with scaling factors outside the above mentioned interval are retested until they bear scaling factors inside the above mentioned interval. The above method produces a scaling of the y-values without altering the shape of the curve. The reference compound used is 2-[4-amino-2-ethoxymethyl-6,7,8,9-tetrahydro-α,α-dimethyl-1H-imidazo[4,5-c]quinolin-1-yl]ethanol hydrate (U.S. Pat. No. 5,352,784; Example 91). The median expected area is the median area across all plates that are part of a given experiment.

A second scaling may also be performed to reduce inter-experiment variability (across multiple experiments). All background-subtracted values are multiplied by a single adjustment ratio to decrease experiment-to-experiment variability. The adjustment ratio is the area of the reference compound in the new experiment divided by the expected area of the reference compound based on an average of previous experiments (unadjusted readings). This results in the scaling of the reading (y-axis) for the new data without changing the shape of the dose-response curve. The reference compound used is 2-[4-amino-2-ethoxymethyl-6,7,8,9-tetrahydro-α,α-dimethyl-1H-imidazo[4,5-c]quinolin-1-yl]ethanol hydrate (U.S. Pat. No. 5,352,784; Example 91) and the expected area is the sum of the median dose values from an average of previous experiments.

The minimum effective concentration is calculated based on the background-subtracted, reference-adjusted results for a given experiment and compound. The minimum effective concentration (μmolar) is the lowest of the tested compound concentrations that induces a response over a fixed cytokine concentration for the tested cytokine (usually 20 pg/mL for IFN-α and 40 pg/mL for TNF-α). The maximal response is the maximal amount of cytokine (pg/ml) produced in the dose-response.

### TNF-α Inhibition in Mouse Cells

Certain compounds of the invention may modulate cytokine biosynthesis by inhibiting production of tumor necrosis factor α (TNF-α) when tested using the method described below.

The mouse macrophage cell line Raw 264.7 is used to assess the ability of compounds to inhibit tumor necrosis factor-α (TNF-α) production upon stimulation by lipopolysaccharide (LPS).

Single Concentration Assay:

Blood Cell Preparation for Culture

Raw cells (ATCC) are harvested by gentle scraping and then counted. The cell suspension is brought to 3×10⁵ cells/mL in RPMI with 10% fetal bovine serum (FBS). Cell suspension (100 μL) is added to 96-well flat bottom sterile tissues culture plates (Becton Dickinson Labware, Lincoln Park, N.J.). The final concentration of cells is 3×10⁴ cells/well. The plates are incubated for 3 hours. Prior to the addition of test compound the medium is replaced with colorless RPMI medium with 3% FBS.

Compound Preparation

The compounds are solubilized in dimethyl sulfoxide (DMSO). The DMSO concentration should not exceed a final concentration of 1% for addition to the culture wells. Compounds are tested at 5 μM. LPS (Lipopolysaccaride from Salmonella typhimurium, Sigma-Aldrich) is diluted with colorless RPMI to the EC₇₀ concentration as measured by a dose response assay.

Incubation

A solution of test compound (1 μl) is added to each well. The plates are mixed on a microtiter plate shaker for 1 minute and then placed in an incubator. Twenty minutes later the solution of LPS (1 μL, EC₇₀ concentration ˜10 ng/ml) is added and the plates are mixed for 1 minute on a shaker. The plates are incubated for 18 to 24 hours at 37° C. in a 5% carbon dioxide atmosphere.

TNF-α Analysis

Following the incubation the supernatant is removed with a pipet. TNF-α concentration is determined by ELISA using a mouse TNF-α kit (from Biosource International, Camarillo, Calif.). Results are expressed in pg/mL. TNF-α expression upon LPS stimulation alone is considered a 100% response.

Dose Response Assay:

Blood Cell Preparation for Culture

Raw cells (ATCC) are harvested by gentle scraping and then counted. The cell suspension is brought to 4×10⁵ cells/mL in RPMI with 10% FBS. Cell suspension (250 μL) is added to 48-well flat bottom sterile tissues culture plates (Costar, Cambridge, Mass.). The final concentration of cells is 1×10⁵ cells/well. The plates are incubated for 3 hours. Prior to the addition of test compound the medium is replaced with colorless RPMI medium with 3% FBS.

Compound Preparation

The compounds are solubilized in dimethyl sulfoxide (DMSO). The DMSO concentration should not exceed a final concentration of 1% for addition to the culture wells. Compounds are tested at 0.03, 0.1, 0.3, 1, 3, 5 and 10 μM. LPS (Lipopolysaccaride from Salmonella typhimurium, Sigma-Aldrich) is diluted with colorless RPMI to the EC₇₀ concentration as measured by dose response assay.

Incubation

A solution of test compound (200 μl) is added to each well. The plates are mixed on a microtiter plate shaker for 1 minute and then placed in an incubator. Twenty minutes later the solution of LPS (200 μL, EC₇₀ concentration ˜10 ng/ml) is added and the plates are mixed for 1 minute on a shaker. The plates are incubated for 18 to 24 hours at 37° C. in a 5% carbon dioxide atmosphere.

TNF-α Analysis

Following the incubation the supernatant is removed with a pipet. TNF-α concentration is determined by ELISA using a mouse TNF-α kit (from Biosource International, Camarillo, Calif.). Results are expressed in pg/mL. TNF-α expression upon LPS stimulation alone is considered a 100% response.

The complete disclosures of the patents, patent documents, and publications cited herein are incorporated by reference in their entirety as if each were individually incorporated. Various modifications and alterations to this invention will become apparent to those skilled in the art without departing from the scope and spirit of this invention. It should be understood that this invention is not intended to be unduly limited by the illustrative embodiments and examples set forth herein and that such examples and embodiments are presented by way of example only with the scope of the invention intended to be limited only by the claims set forth herein as follows.

1. A compound of the formula (I); 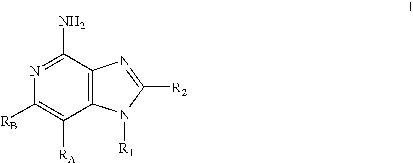
wherein:
R₂ is selected from the group consisting of:
—X′—N(R₈ₐ)—C(R₆)—N(R₈ₐ)—W—R₂₋₁,
—X′—N(R₈ₐ)—C(R₆)—N(OR₈ₐ)—R₂₋₁, 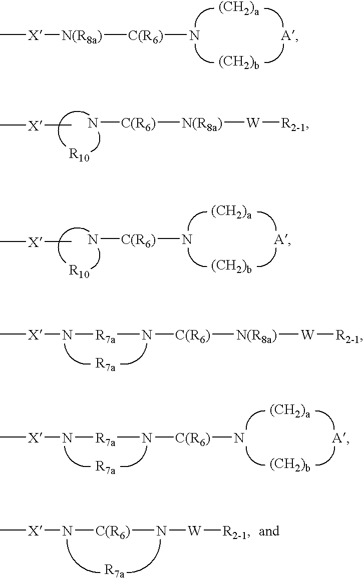
—X′—N(R₈ₐ)—C(R₆)—O—R₂₋₁;
X′ is selected from the group consisting of C₁₋₄ alkylene and C₂₋₄ alkenylene;
R₂₋₁ is selected from the group consisting of hydrogen, C₁₋₄ alkyl, C₂₋₄ alkenyl, C₂₋₄ alkynyl, aryl, arylC₁₋₄ alkylenyl, aryloxyC₁₋₄ alkylenyl, C₁₋₄ alkylarylenyl, heteroaryl, heteroarylC₁₋₄ alkylenyl, heteroaryloxyC₁₋₄ alkylenyl, CJ 4 alkylheteroarylenyl, and heterocyclyl wherein the C₁₋₄ alkyl, C₂₋₄ alkenyl, C₁₋₂₄ alkynyl, aryl, arylC₁₋₄ alkylenyl, aryloxyC₁₋₄alkylenyl, C₁₋₄ alkylarylenyl, heteroaryl, heteroarylC₁₋₄alkylenyl, heteroaryloxyC₁₋₄ alkylenyl, C₁₋₄ alkylheteroarylenyl, and heterocyclyl groups are unsubstituted or substituted by one or more substituents independently selected from the group consisting of C₁₋₄ alkyl, C₁₋₄ alkoxy, C₁₋₄ alkanoyl, C₁₋₄ alkoxycarbonyl, hydroxyC₁₋₄ alkylenyl, haloC₁₋₄ alkylenyl, haloC₁₋₄ alkyleneoxy, halogen, nitro, hydroxy, mercapto, cyano, amino, C₁₋₄ alkylamino, di(C₁₋₄ alkyl)amino, and in the case of C₁₋₄ alkyl, C₂₋₄ alkenyl, C₂₋₄ alkynyl, and heterocyclyl, oxo;
A′ is selected from the group consisting of —O—, —C(O)—, —CH₂—, —S(O)₀₋₂—, —NH—, and —N(C₁₋₄ alkyl)-;
R₇ₐ is C₂₋₄ alkylene;
R₈ₐ is selected from the group consisting of hydrogen and C₁₋₄ alkyl;
R_{A} and R_{B} are independently selected from the group consisting of:
hydrogen,
halogen,
alkyl,
alkenyl,
alkoxy,
alkylthio, and
—N(R₉)₂;
or R_{A} and R_{B} taken together form either a fused aryl ring that is unsubstituted or substituted by one or more Rₐ groups, or a fused 5 to 7 membered saturated ring that is unsubstituted or substituted by one or more R_{c} groups;
or R_{A} and R_{B} taken together form a fused heteroaryl or 5 to 7 membered saturated ring containing one heteroatom selected from the group consisting of N and S, wherein the heteroaryl ring is unsubstituted or substituted by one or more R_{b} groups, and the 5 to 7 membered saturated ring is unsubstituted or substituted by one or more R_{b} groups;
Rₐ is selected from the group consisting of halogen, alkyl, haloalkyl, alkoxy, and —N(R₉)₂;
R_{b} is selected from the group consisting of halogen, hydroxy, alkyl, haloalkyl, alkoxy, and —N(R₉)—;
R_{c} is selected from the group consisting of halogen, hydroxy, alkyl, alkenyl, haloalkyl, alkoxy, alkylthio, and —N(R₉)₂;
R₁ is selected from the group consisting of:
—R₄,
—X—Y—R₄,
—X—Y—X—Y—R₄, and
—X—R₅;
X is selected from the group consisting of alkylene, arylene, heteroarylene, and heterocyclylene wherein the alkylene group can be optionally interrupted or terminated by arylene, heteroarylene or heterocyclylene and optionally interrupted by one or more —O— groups;
Y is selected from the group consisting of:
—S(O)₀₋₂—,
—C(R₆)—,
—C(R₆)—O—,
—O—C(R₆)—,
—O—C(O)—O—,
—N(R₈)-Q-,
—O—C(R₆)—N(R₈)—,
—C(R₆)—N(OR₉)—, 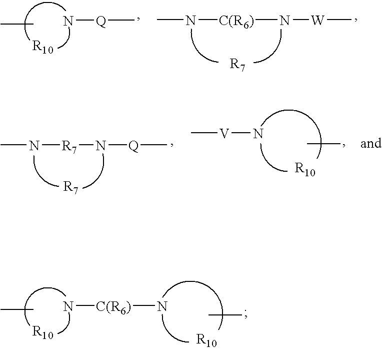
R₄ is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the alkyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups can be unsubstituted or substituted by one or more substituents independently selected from the group consisting of alkyl, alkoxy, hydroxyalkylenyl, haloalkylenyl, haloalkyleneoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, (dialkylamino)alkyleneoxy, and in the case of alkyl and heterocyclyl, oxo;
R₅ is selected from the group consisting of: 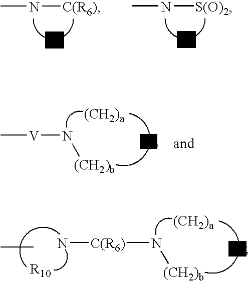
R₆ is selected from the group consisting of ═O and ═S;
R₇ is C₂₋₇ alkylene;
R₉ is selected from the group consisting of hydrogen, alkyl, alkoxyalkylenyl, and arylalkylenyl;
R₉ is selected from the group consisting of hydrogen and alkyl;
R₁₀ is C₃₋₈ alkylene;
A is selected from the group consisting of —O—, —C(O)—, —CH₂—, —S(O)₀₋₂—, and —N(R₄)—;
Q is selected from the group consisting of a bond, —C(R₆)—, —C(R₆)—C(R₆)—, —S(O)₂—, —C(R₆)—N(R₈)—W—, —S(O)₂—N(R₈)—, —C(R₆)—O—, —C(R₆)—S—, and —C(R₆)—N(OR₉)—;
V is selected from the group consisting of —O—C(R₆)— and —N(R₈)—C(R₆)—;
W is selected from the group consisting of a bond, —C(O)—, and —S(O)₂—; and
a and b are independently integers from 1 to 6 with the proviso that a+b is ≦7;
with the proviso that when R_{A} and R_{B} taken together for a ring, and X is interrupted with one —O— group, then Y is other than —S(O)₀₋₂—; and
with the further proviso that when R_{A} and R_{B} are independently hydrogen, halogen, alkyl, alkenyl, alkoxy, alkylthio, or —N(R₉)₂, and R₂ is selected from the group consisting of: 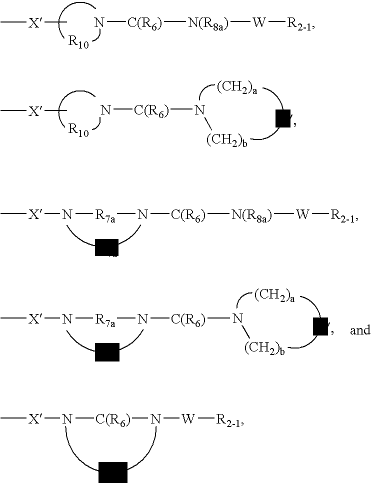
then X is not interrupted with one or more —O— groups and Y is other than —S(O)₀₋₂—;
or a pharmaceutically acceptable salt thereof.

2. (canceled)

3. The compound or salt of claim 1 wherein the compound is of the following formula (III): 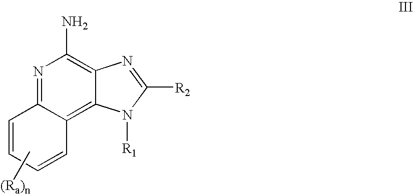
wherein:
R₂ is selected from the group consisting of:
—X′—N(R₈ₐ)—C(R₆)—N(R₈ₐ)—W—R₂₋₁,
—X′—N(R₈ₐ)—C(R₆)—N(OR₈ₐ)—R₂₋₁, 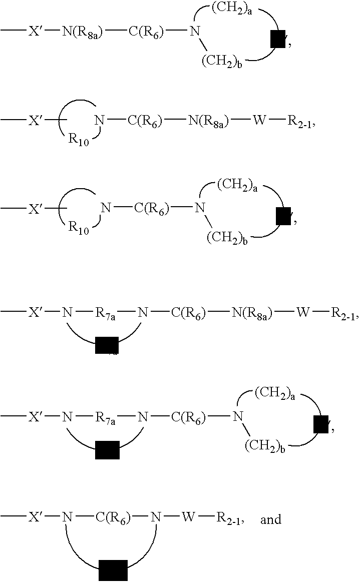
—X′—N(R₈ₐ)—C(R₆)—O—R₂₋₁;
X′ is selected from the group consisting of C₁₋₄ alkylene and C₂₋₄ alkenylene;
R₂₋₁ is selected from the group consisting of hydrogen, C₁₋₄ alkyl, C₂₋₄ alkenyl, C₂₋₄ alkynyl, aryl, arylC₁₋₄ alkylenyl, aryloxyC₁₋₄ alkylenyl, C₁₋₄alkylarylenyl, heteroaryl, heteroarylC₁₋₄ alkylenyl, heteroaryloxyC₁₋₄ alkylenyl, C₁₋₄ alkylheteroarylenyl, and heterocyclyl wherein the C₁₋₄ alkyl, C₂₋₄ alkenyl, C₂₋₄ alkynyl, aryl, arylC₁₋₄alkylenyl, aryloxyC₁₋₄ alkylenyl, C₁₋₄ alkylarylenyl, heteroaryl, heteroarylC₁₋₄ alkylenyl, heteroaryloxyC₁₋₄ alkylenyl, C₁₋₄ alkylheteroarylenyl, and heterocyclyl groups are unsubstituted or substituted by one or more substituents independently selected from the group consisting of C₁₋₄ alkyl, C₁₋₄ alkoxy, C₁₋₄ alkanoyl, C₁₋₄ alkoxycarbonyl, hydroxyC₁₋₄ alkylenyl, haloC₁₋₄ alkylenyl, haloC₁₋₄ alkyleneoxy, halogen, nitro, hydroxy, mercapto, cyano, amino, C₁₋₄ alkylamino, di(C₁₋₄ alkyl)amino, and in the case of C₁₋₄ alkyl, C₂₋₄ alkenyl, C₂₋₄ alkynyl, and heterocyclyl, oxo;
A′ is selected from the group consisting of —O—, —C(O)—, —CH₂—, —S(O)₀₋₂—, —NH—, and —N(C₁₋₄ alkyl)-;
R₇ₐ is C₂₋₄ alkylene;
R₈ₐ is selected from the group consisting of hydrogen and C₁₋₄ allyl;
Rₐ is selected from the group consisting of halogen, alkyl, haloalkyl, alkoxy, and —N(R₉)₂;
n is an integer from 0 to 4;
R₁ is selected from the group consisting of:
—R₄,
—X—Y—R₄,
—X—Y—X—Y—R₄, and
—X—R₅;
X is selected from the group consisting of alkylene, arylene, heteroarylene, and heterocyclylene wherein the alkylene group can be optionally interrupted or terminated by arylene, heteroarylene or heterocyclylene and optionally interrupted by one or more —O— groups;
Y is selected from the group consisting of:
—S(O)O₀₋₂—,
—C(R₆)—,
—C(R₆)—O—,
—O—C(R₆)—,
—O—C(O)—O—,
—N(R₈)-Q-,
—O—C(R₆)—N(R₈)—,
—C(R₆)—N(OR₉)—, 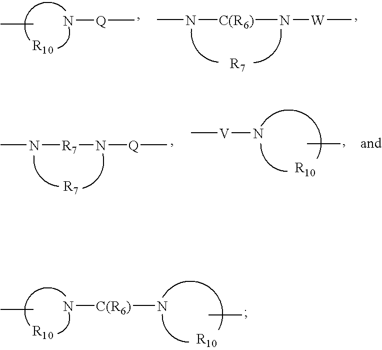
R₄ is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the alkyl, aryl, arylalkylenyl, aryloxyalkylenyl, alkylarylenyl, heteroaryl, heteroarylalkylenyl, heteroaryloxyalkylenyl, alkylheteroarylenyl, and heterocyclycl groups can be unsubstituted or substituted by one or more substituents independently selected from the group consisting of alkyl, alkoxy, hydroxyalkylenyl, haloalkylenyl, haloalkyleneoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, (dialkylamino)alkyleneoxy, and in the case of alkyl and heterocyclyl, oxo;
R₅ is selected from the group consisting of: 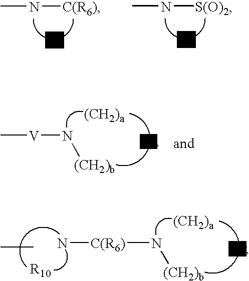
R₆ is selected from the group consisting of ═O and ═S;
R₇ is C₂₋₇ alkylene;
R₈ is selected from the group consisting of hydrogen, alkyl, alkoxyalkylenyl, and arylalkylenyl;
R₉ is selected from the group consisting of hydrogen and alkyl;
R₁₀ is C₃₋₈ alkylene;
A is selected from the group consisting of —O—, —C(O)—, —CH₂—, —S(O)₀₋₂—, and —N(R₄)—;
Q is selected from the group consisting of a bond, —C(R₆)—, —C(R₆)—C(R₆)—, —S(O)₂—, —C(R₆)—N(R₈)—W—, —S(O)₂—N(R₈)—, —C(R₆)—O—, —C(R₆)—S—, and —C(R₆)—N(OR₉)—;
V is selected from the group consisting of —O—C(R₆)— and —N(R₈)—C(R₆)—;
W is selected from the group consisting of a bond, —C(O)—, and —S(O)₂—; and
a and b are independently integers from 1 to 6 with the proviso that a+b is ≦7;
with the proviso that when X is interrupted with one —O— group, then Y is other than —S(O)₀₋₂—;
or a pharmaceutically acceptable salt thereof.

4-5. (canceled)

6. The compound or salt of claim 1 wherein the ring formed by R_{A} and R_{B} is unsubstituted.

7. (canceled)

8. The compound or salt of claim 3 wherein n is 0.

9-13. (canceled)

14. The compound or salt of claim 1 wherein R₂ is selected from the group consisting of:
—X′—N(R₈ₐ)—C(R₆)—N(R₈ₐ)—W—R₂₋₁,
—X′—N(R₈ₐ)—C(R₆)—N(OR₈ₐ)—W—R₂₋₁, and 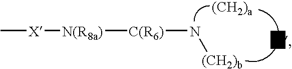
wherein a and b are independently integers from 1 to 4 with the proviso that a+b is ≦5.

15. The compound or salt of claim 14 wherein R₂ is —X′—N(R₈ₐ)C(R₆)—N(R₈ₐ)—W—R₂₋₁—.

16. (canceled)

17. The compound or salt of claim 1 wherein R₂₋₁ is selected from the group consisting of C₁₋₄ alkyl, aryl, or substituted aryl wherein the substituent is C₁₋₄ alkyl, C₁₋₄ alkoxy, or halogen.

18. The compound or salt of claim 1 wherein W is a bond, and R₂₋₁ is selected from the group consisting of C₁₋₄ alkyl, phenyl, or substituted phenyl wherein the substituent is C₁₋₄ alkyl, C₁₋₄ alkoxy, or halogen.

19. The compound or salt of claim 1 wherein W is a bond, and R₂₋₁ is selected from the group consisting of hydrogen, methyl, and ethyl.

20. A compound of the formula (X): 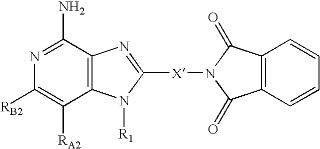
wherein:
X′ is selected from the group consisting of C₁₋₄ alkylene and C₂₋₄ alkenylene;
R_{A2} and R_{B2} taken together form either a fused aryl ring that is unsubstituted or substituted by one or more Rₐ groups, or a fused 5 to 7 membered saturated ring that is unsubstituted or substituted by one or more R_{c} groups;
or R_{A2} and R_{B2} taken together form a fused heteroaryl or 5 to 7 membered saturated ring containing one heteroatom selected from the group consisting of N and S, wherein the heteroaryl ring is unsubstituted or substituted by one or more R_{b} groups, and the 5 to 7 membered saturated ring is unsubstituted or substituted by one or more R_{c} groups;
Rₐ is selected from the group consisting of halogen, alkyl, haloalkyl, alkoxy, and —N(R₉)₂;
R_{b} is selected from the group consisting of halogen, hydroxy, alkyl, haloalkyl, alkoxy, and —N(R₉)₂;
R_{c} is selected from the group consisting of halogen, hydroxy, alkyl, alkenyl, haloalkyl, alkoxy, alkylthio, and —N(R₉)₂;
R₁ is selected from the group consisting of:
—R₄,
—X—R₄,
—X—Y—R₄,
—X—Y—X—Y—R₄, and
—X—R₅;
X is selected from the group consisting of alkylene, arylene, heteroarylene, and heterocyclylene wherein the alkylene group can be optionally interrupted or terminated by arylene, heteroarylene or heterocyclylene and optionally interrupted by one or more —O— groups;
Y is selected from the group consisting of:
—S(O)₀₋₂—,
—C(R₆)—,
—C(R₆)—O—,
O—C(R₆)—,
—O—C(O)—O—,
—N(R₈)-Q-,
—O—C(R₆)—N(R₈)—,
—C(R₆)—N(OR₉)—, 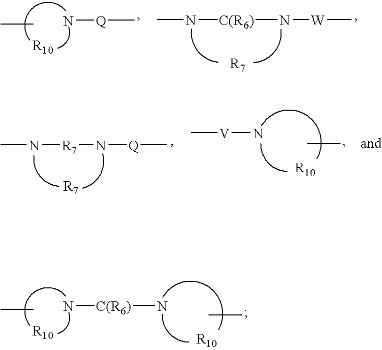
R₄ is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the alkyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups can be unsubstituted or substituted by one or more substituents independently selected from the group consisting of alkyl, alkoxy, hydroxyalkylenyl, haloalkylenyl, haloalkyleneoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, (dialkylamino)alkyleneoxy, and in the case of alkyl and heterocyclyl, oxo;
R₅ is selected from the group consisting of: 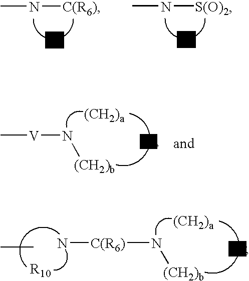
R₆ is selected from the group consisting of ═O and ═S;
R₇ is C₂₋₇ alkylene;
R₈ is selected from the group consisting of hydrogen, alkyl, alkoxyalkylenyl, and arylalkylenyl;
R₉ is selected from the group consisting of hydrogen and alkyl;
R₁₀ is C₃₋₈ alkylene;
A is selected from the group consisting of —O—, —C(O)—, —CH₂—, —S(O)₀₋₂—, and —N(R₄)—;
Q is selected from the group consisting of a bond, —C(R₆)—, —C(R₆)—C(R₆)—, —S(O)₂—, —C(R₆)—N(R₈)—W—, —S(O)₂—N(R₈)—, —C(R₆)—O—, —C(R₆)—S—, and —C(R₆)—N(OR₉)—;
V is selected from the group consisting of —O—C(R₆)— and —N(R₈)—C(R₆)—;
W is selected from the group consisting of a bond, —C(O)—, and —S(O)₂—; and
a and b are independently integers from 1 to 6 with the proviso that a+b is ≦7; or a pharmaceutically acceptable salt thereof.

21-25. (canceled)

26. The compound or salt of claim 1 wherein R₁ is selected from the group consisting of alkyl, arylalkylenyl, aryloxyalkylenyl, hydroxyalkylenyl, aminoalkylenyl, haloalkylenyl, alkylsulfonylalkylenyl, —X—Y—R₄, and —X—R₅; wherein X is alkylene; Y is —N(R₈)—C(O)—, —N(R₈)—S(O)₂—, —N(R₈)—C(O)—N(R₈)—, —N(R₈)—C(S)—N(R₈)—, or —N(R₈)—S(O)₂—N(R₈)—; R₄ is alkyl, aryl, or heteroaryl; and R₅ is

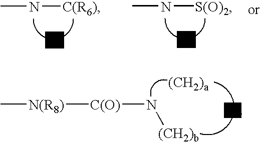

27. The compound or salt of claim 26 wherein R₁ is alkyl or hydroxyalkylenyl.

28. The compound or salt of claim 1 wherein R₁ is selected from the group consisting of:
C₁₋₁₀ alkyl,
hydroxyC₁₋₆ alkylenyl,
C₁₋₄ alkyl-O—C₁₋₆ alkylenyl,
phenyl-C₁₋₄ alkylenyl, and
phenyl;
wherein phenyl is unsubstituted or substituted with one or two substituents selected from the group consisting of C₁₋₄ alkyl, C₁₋₄alkoxy, and halogen.

29-30. (canceled)

31. The compound or salt of claim 1 wherein X′ is C₁₋₄ alkylene.

32. The compound or salt of claim 31 wherein X′ is methylene or ethylene.

33. A pharmaceutical composition comprising a therapeutically effective amount of a compound or salt of claim 1 and a pharmaceutically acceptable carrier.

34. A method of inducing cytokine biosynthesis in an animal comprising administering an effective amount of a compound or salt of claim 1 to the animal.

35. A method of treating a viral disease in an animal in need thereof comprising administering a therapeutically effective amount of a compound or salt of claim 1 to the animal.

36. A method of treating a neoplastic disease in an animal in need thereof comprising administering a therapeutically effective amount of a compound or salt of claim 1 to the animal.

37-38. (canceled)

39. A compound of the formula (XVIII): 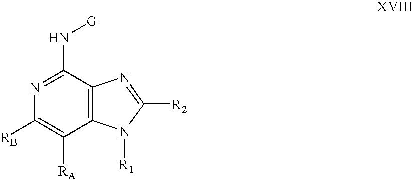
wherein:
R₂ is selected from the group consisting of:
—X′—N(R₈ₐ)—C(R₆)—N(R₈ₐ)—W—R₂₋₁,
—X′—N(R₈ₐ)—C(R₆)—N(OR₈ₐ)—R₂₋₁, 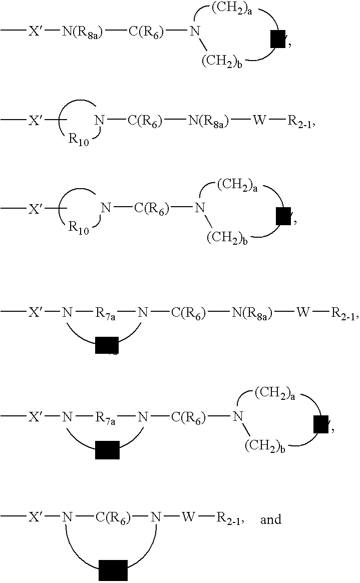
—X′—N(R₈ₐ)—C(R₆)—O—R₂₋₁—;
X′ is selected from the group consisting of C₁₋₄ alkylene and C₂₋₄ alkenylene;
R₂₋₁ is selected from the group consisting of hydrogen, C₁₋₄ alkyl, C₂₋₄ alkenyl, C₂₋₄ alkynyl, aryl, arylC₁₋₄ alkylenyl, aryloxyC₁₋₄ alkylenyl, C₁₋₄ alkylarylenyl, heteroaryl, heteroarylC₁₋₄ alkylenyl, heteroaryloxyC₁₋₄ alkylenyl, C₁₋₄ alkylheteroarylenyl, and heterocyclyl wherein the C₁₋₄ alkyl, C₂₋₄ alkenyl, C₂₋₄ alkynyl, aryl, arylC₁₋₄ alkylenyl, aryloxyC₁₋₄ alkylenyl, C₁₋₄ alkylarylenyl, heteroaryl, heteroarylC₁₋₄ alkylenyl, heteroaryloxyC₁₋₄ alkylenyl, C₁₋₄ alkylheteroarylenyl, and heterocyclyl groups are unsubstituted or substituted by one or more substituents independently selected from the group consisting of C₁₋₄ alkyl, C₁₋₄ alkoxy, C₁₋₄ alkanoyl, C₁₋₄ alkoxycarbonyl, hydroxyC₁₋₄ alkylenyl, haloC₁₋₄ alkylenyl, haloC₁₋₄ alkyleneoxy, halogen, nitro, hydroxy, mercapto, cyano, amino, C₁₋₄ alkylamino, di(C₁₋₄ alkyl)amino, and in the case of C₁₋₄ alkyl, C₂₋₄ alkenyl, C₂₋₄ alkynyl, and heterocyclyl, oxo;
A′ is selected from the group consisting of —O—, —C(O)—, —CH₂—, —S(O)₀₋₂—, —NH—, and —N(C₁₋₄ alkyl)-;
R₇ₐ is C₂₋₄alkylene;
R₈ₐ selected from the group consisting of hydrogen and C₁₋₄ alkyl;
G is selected from the group consisting of:
—C(O)—R′,
α-aminoacyl,
α-aminoacyl-α-aminoacyl,
—C(O)—O—R′,
—C(O)—N(R″)R′,
—C(═NY′)—R′,
—CH(OH)—C(O)—OY′,
—CH(OC₁₋₄ alkyl)Y₀,
—CH₂Y₁, and
—CH(CH₃)Y₁;
R′ and R″ are independently selected from the group consisting of C₁₋₁₀ alkyl, C₃₋₇ cycloalkyl, and benzyl, each of which may be unsubstituted or substituted by one or more substitutents selected from the group consisting of halogen, hydroxy, nitro, cyano, carboxy, C₁₋₆ alkyl, C₁₋₄ alkoxy, aryl, heteroaryl, arylC₁₋₄ alkylenyl, heteroarylC₁₋₄ alkylenyl, haloC₁₋₄ alkyl, haloC₁₋₄ alkoxy, —O—C(O)—CH₃, —C(O)—O—CH₃, —C(O)—NH₂, —O—CH₂—C(O)—NH₂, —NH₁₂, and —S(O)₂—NH₂;
α-aminoacyl is an acyl group derived from an amino acid selected from the group consisting of the naturally occurring L-amino acids;
Y′ is selected from the group consisting of hydrogen, C₁₋₆alkyl, and benzyl;
Y₀ is selected from the group consisting of C₁₋₆alkyl, carboxyC₁₋₆alkyl, aminoC₁₋₄ alkyl, mono-N—C₁₋₆ alkylaminoC₁₋₄ alkyl, and di-N,N—C₁₋₆ alkylaminoC₁₋₄ alkyl;
Y₁ is selected from the group consisting of mono-N—C₁₋₁₆ alkylamino, di-N,N—C₁₋₆ alkylamino, morpholin-4-yl, piperidin-1-yl, pyrrolidin-1-yl, and 4-C₁₋₄ alkylpiperazin-1-yl;
R_{A} and R_{B} are independently selected from the group consisting of:
hydrogen,
halogen,
alkyl,
alkenyl,
alkoxy,
alkylthio, and
—N(R₉)₂;
or R_{A} and R_{B} taken together form either a fused aryl ring that is unsubstituted or substituted by one or more Rₐ groups, or a fused 5 to 7 membered saturated ring that is unsubstituted or substituted by one or more R_{c} groups;
or R_{A} and R_{B} taken together form a fused heteroaryl or 5 to 7 membered saturated ring containing one heteroatom selected from the group consisting of N and S, wherein the heteroaryl ring is unsubstituted or substituted by one or more R_{b} groups, and the 5 to 7 membered saturated ring is unsubstituted or substituted by one or more R_{c} groups;
Rₐ is selected from the group consisting of halogen, alkyl, haloalkyl, alkoxy, and —N(R₉)₂;
R_{b} is selected from the group consisting of halogen, hydroxy, alkyl, haloalkyl, alkoxy, and —N(R₉)₂;
R_{c} is selected from the group consisting of halogen, hydroxy, alkyl, alkenyl, haloalkyl, alkoxy, alkylthio, and —N(R₉)₂;
R₁ is selected from the Group consisting of:
—R₄,
—X—R₄,
—X—Y—R₄,
—X—Y—X—Y—R₄, and
—X—R₅;
X is selected from the group consisting of alkylene, arylene, heteroarylene, and heterocyclylene wherein the alkylene group can be optionally interrupted or terminated by arylene, heteroarylene or heterocyclylene and optionally interrupted by one or more —O— groups;
Y is selected from the group consisting of:
—S(O)₀₋₂—,
—C(R₆)—,
—C(R₆)—O—,
—O—C(R₆)—,
—O—C(O)—O—,
—N(R₈)-Q-,
—O—C(R₆)—N(R₈)—,
—C(R₆)—N(OR₉)—, 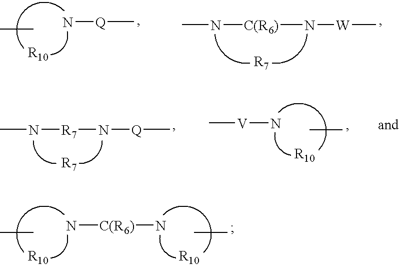
R₄ is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the alkyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups can be unsubstituted or substituted by one or more substituents independently selected from the group consisting of alkyl, alkoxy, hydroxyalkylenyl, habalkylenyl, haloalkyleneoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, (dialkylamino)alkyleneoxy, and in the case of alkyl and heterocyclyl, oxo;
R₅ is selected from the group consisting of: 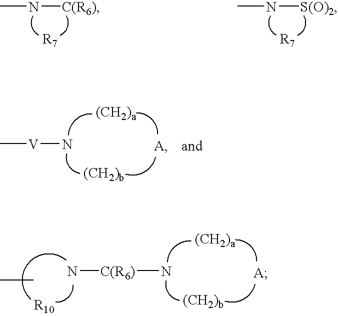
R₆ is selected from the group consisting of ═O and ═S;
R₇ is C₂₋₇ alkylene;
R₈ is selected from the group consisting of hydrogen, alkyl, alkoxyalkylenyl, and arylalkylenyl;
R₉ is selected from the group consisting of hydrogen and alkyl;
R₁₀ is C₃₋₈ alkylene;
A is selected from the group consisting of —O—, —C(O)—, —CH₂—, —S(O)₀₋₂—, and —N(R₄)—;
Q is selected from the group consisting of a bond, —C(R₆)—, —C(R₆)—C(R₆)—, —S(O)₂—, —C(R₆)—N(R₈)—W—, —S(O)₂—N(R₈)—, —C(R₆)—O—, —C(R₆)—S—, and —C(R₆)—N(OR₉)—;
V is selected from the group consisting of —O—C(R₆)— and —N(R₈)—C(R₅)—;
W is selected from the group consisting of a bond, —C(O)—, and —S(O)₂—; and
a and b are independently integers from 1 to 6 with the proviso that a+b is ≦7;
with the proviso that when R_{A} and R_{B} taken together form a ring, and X is interrupted with one —O— group, then Y is other than —S(O)₀₋₂—; and
with the further proviso that when R_{A} and R_{B} are independently hydrogen, halogen, alkyl, alkenyl, alkoxy, alkylthio, or —N(R₉)₂, and R₂ is selected from the group consisting of: 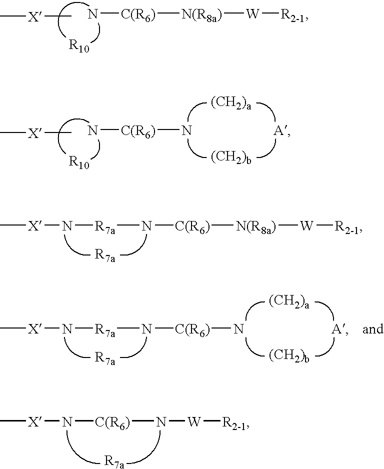
then X is not interrupted with one or more —O— groups and Y is other than —S(O)₀₋₂—;
or a pharmaceutically acceptable salt thereof.
